(19) United States
(12) Patent Application Publication
(10) Pub. No.: US 2024/0158871 A1
(43) Pub. Date: May 16, 2024

(54) Pan-Cancer Classification Based on FMRP Pathway Activity that Informs Differential Prognosis and Therapeutic Responses

(71) Applicant: ECOLE POLYTECHNIQUE FEDERALE DE LAUSANNE (EPFL), Lausanne (CH)

(72) Inventors: Sadeq Saqafi, Lausanne (CH); Douglas Hanahan, Lausanne (CH)

(21) Appl. No.: 18/516,274

(22) Filed: Nov. 21, 2023

Related U.S. Application Data
(63) Continuation of application No. PCT/EP2022/063807, filed on May 20, 2022.
(60) Provisional application No. 63/191,377, filed on May 21, 2021.

(51) Int. Cl. C12Q 1/6886; C12Q 1/6874

(52) U.S. Cl. CPC C12Q 1/6886; C12Q 1/6874; C12Q 2600/106; C12Q 2600/158; C12Q 2600/166

(57) ABSTRACT

The present invention relates to methods and compositions which provide a companion diagnostic for cancer therapy. A method for identifying and stratifying a patient or group of patients with cancer as (i) being high or low for FMRP activity, (ii) having a high or low risk prognosis, and/or (iii) being a responder or non-responder to cancer therapy, and/or (iv) having high or low immune cell infiltrated tumor.

### BACKGROUND

The role of upregulated fragile X mental retardation protein (FMRP) protein in cancer cells has been previously shown (see e.g., US 2020-0354718), wherein upregulated FMRP activity suppresses the immune response against tumors. Genetic ablation of the FMR1 gene, which encodes FMRP, releases the immunosuppression and activates CD8 T-cell mediated tumor immunity in mouse models, resulting in tumor shrinkage and extended survival compared to otherwise isogenic FMRP-expressing tumors.

Despite the demonstrable role of FMRP in tumor progression and shaping an immuno-suppressive tumor micro-environment, assessing its functional activity in tumor samples has proven to be challenging. Because of multiple post-transcriptional and translational modifications of FMR1 mRNA and FMRP protein, respectively, the level of FMR1 mRNA expression and of FMRP protein expression are not good biomarkers of the endogenous immuno-suppressive activity of this protein.

Thus, there is a need for improved methods for assessing FMRP activity in tumors and determining the likelihood that a cancer can be successfully treated by a variety of cancer therapies whose efficacy is dependent upon, or limited by, FMRP pathway activity.

### SUMMARY OF THE INVENTION

The present invention relates to methods and compositions which provide a companion diagnostic for cancer therapy. In particular, the invention relates to methods and reagents for determining the likelihood that a cancer can be successfully treated by cancer therapies whose efficacy is dependent upon, or limited by, FMRP pathway activity. The methods and compositions of this invention are useful for separating cancer patients as potential responders from non-responders to cancer therapy. The invention is based, at least in part, on the discovery that treatment with a cancer therapy is likely to be more effective when a patient's FMRP activity score is considered.

### DESCRIPTION OF THE DRAWINGS

FIG. 1A through FIG. 1H show patient classification across 31 different cancer types, based on FMR1 mRNA expression (panels A and B), which is not informative, in contrast to the newly invented FMRP pathway-activity signature scoring system (FMRP-activity Pan-Signature: panels C and D; Sub-Signature 1: Panels E and F; Sub-Signature 3: Panels G and H), which it is informative and statistically significant for all. Each panel shows the association (or not) with patient prognosis (A, C, E, and G: overall survival; B, D, F, and H: progression-free survival). The COX-model was used considering the tumor type as covariate to estimate the significance of correlation. The data used in this figure were downloaded from the latest TCGA PanCan Atlas.

FIG. 2A through FIG. 2C depict FMRP-activity score in breast cancer. A. The FMRP-activity score shows the highest level in the basal-like subtype, which is the most aggressive subtype of breast cancer. Only up-regulated genes in Pan-Signature 1 were used to derive the signature scores for this panel. B. The FMRP-activity score (Pan-Signature) correlates with overall survival for all breast cancer patients. C. The FMRP-activity score (Pan-Signature) specifically correlates with overall survival for the Luminal A subtype of breast cancer patients. The data used in this figure were downloaded from the latest breast cancer cohort of TCGA PanCan Atlas.

FIG. 3A through FIG. 3C depict FMRP-activity score (Pan-Signature) in colorectal carcinoma. A. FMRP-activity score correlation with overall survival for all colorectal cancer patients. B. FMRP-activity score correlation with overall survival for microsatellite stable (MSS) colorectal cancer patients. C. FMRP-activity score lack of correlation with overall survival for microsatellite instable (MSI) colorectal cancer patients. The data used in this figure were downloaded from the latest colorectal cancer cohort of TCGA PanCan Atlas.

FIG. 4A through FIG. 4D depict FMRP-activity score correlation with immune-checkpoint inhibitor therapy response in cancer patients. FIG. 4A. FMRP-activity score correlation with overall survival for melanoma patients receiving anti-PD1 therapy (left panel); non-responders to anti-PD1 therapy show a higher level of the FMRP-activity score (right panel). FIG. 4B. FMRP-activity score correlation with overall survival for lung cancer patients receiving anti-PD1 or anti-PD-L1 therapy (left panel); non-responders to anti-PD1 or anti-PD-therapy show a higher level of the FMRP-activity score (right panel). FIG. 4C. FMRP-activity score correlation with overall survival for urothelial cancer patients receiving anti-PD-L1 therapy (left panel); non-responders to anti-PD-L1 therapy show a higher level of the FMRP-activity score (right panel). Only up-regulated genes in Sub-Signature 1 were used to derive the signature scores for panel A-C. FIG. 4D. FMRP-activity score (Pan-Signature) correlation with overall survival for melanoma patients receiving anti-CTLA4 therapy (left panel); non-responders to anti-CTLA4 therapy show a higher level of the FMRP-activity score (right panel).

FIG. 5A and FIG. 5B depict FMRP-activity score correlation with chemotherapy response in cancer patients. FIG. 5A. FMRP-activity score correlation with disease-free survival for breast cancer patients receiving Taxanes (left panel); notably, the signature scores are independent of tumor aggressiveness (T-stage, right panel), also shown using COX model in survival analysis considering the T-stage as covariate, which therefore reveals that FMRP-activity signature constitutes an independent prognostic marker. FIG. 5B. FMRP-activity score correlation with progression-free survival for lung cancer patients receiving Paclitaxel, Cisplatin, or Carboplatin (left panel); the signature scores are again independent of tumor aggressiveness (T-stage, right panel), constituting an independent prognostic factor. The COX-model was used, considering the T-stage as covariate to estimate the significance of correlation for survival analysis. Only up-regulated genes from Sub-Signature 1 were used to derive the signature scores for all the panels in FIG. 5.

FIG. 6A through FIG. 6N show the non-reproducibility and lack of correlation between previously published FMRP signatures and those described in this invention. FMR1 mRNA expression (FIG. 6A and FIG. 6B), and FMRP network signature (Luca et al., (2013). The fragile X protein binds mRNAs involved in cancer progression and modulates metastasis formation. EMBO Mol. Med. 5, 1523-1536., FIG. 6C and FIG. 6D) correlations with Breast cancer patients' survival are not informative or statistically significant. Each panel shows the association (or not) with patient prognosis (FIG. 6A, FIG. 6C: overall survival; FIG. 6B, FIG. 6D: progression-free survival). FIG. 6E. Genes constituting the FMRP network signature proposed by Rossella Luca et al., 2013 show no significant overlap with Pan-Signature described in this invention. FMR1 mRNA expression (FIG. 6F and FIG. 6G), and FMRP network signature (F. Zalfa et al., (2017). The fragile X mental retardation protein regulates tumor invasiveness-related pathways in melanoma cells. Cell Death Dis. 8, e3169., FIG. 6H and FIG. 6I) correlations with melanoma patients' survival again are not informative or statistically significant. Each panel shows patient prognosis (FIG. 6F, FIG. 6H: overall survival; FIG. 6G, FIG. 6L progression-free survival). FIG. 6J. The genes comprising the FMRP network signature proposed by F. Zalfa et al., 2017 show no significant overlap with Pan-Signature provided in this invention. FMR1 mRNA expression (FIG. 6K and FIG. 6L), and RIPK1 mRNA expression (FIG. 6M and FIG. 6N) correlations with colorectal cancer patients' survival again are not informative or statistically significant. Each panel shows patient prognosis (FIG. 6K, FIG. 6M: overall survival; FIG. 6L, FIG. 6N: progression-free survival).

FIG. 7A through FIG. 7E depict FMRP-activity score (Pan-Signature) in adrenocortical carcinoma, endometrial carcinoma, esophageal adenocarcinoma, pancreatic adenocarcinoma, and liver hepatocellular carcinoma. FIG. 7A-7C show the correlation of the Pan-Signature score with overall-survival (OS, left panels) and progression-free survival (PFS, right panels), in adrenocortical carcinoma (A), endometrial carcinoma (B), and esophageal adenocarcinoma (C). FIG. 7D and FIG. 7E show correlation of the Pan-Signature score with overall-survival in pancreatic adenocarcinoma (D), and liver hepatocellular carcinoma (E).

FIG. 8A through FIG. 8E. FIG. 8A and FIG. 8B demonstrate that FMRP-activity scores are negatively associated both with CD8 T infiltration in multiple human tumors. FIG. 8A shows anti-correlation of the FMRP-activity score (Pan-Signature) with a CD8 T-cell infiltration signature, estimated by the xCell package, in human pan-cancers. Linear regression model with tumor type as covariate was used to estimate the significance of correlation. FIG. 8B shows anti-correlation of the FMRP-activity score (Sub-Signature 1) with a CD8 T-cell infiltration signature, as in FIG. 8A. Only up-regulated genes in FMRP-activity sub-signature 1 were used for deriving the signature score in this analysis. FIG. 8C through FIG. 8E shows no correlation of FMRP-activity scores with tumor grades. FIG. 8C depicts distributions of FMRP Pan-signature scores across different tumors grades in the TCGA human pan-cancer dataset. FIG. 8D depicts distributions of FMRP Sub-signature 1 scores across different tumors grades in the TCGA human pan-cancer dataset. FIG. 8E depicts distributions of FMRP Sub-signature 1 scores, only useing up-regulated genes in the FMRP-activity signature list, across different tumors grades in TCGA human pan-cancer dataset.

FIG. 9A through FIG. 9L. FIG. 9A through FIG. 9C depict anti-correlation of the FMRP-activity score (Pan-Signature) with progression-free survival (PFS, left panels) and CD8 T-cell infiltration signature (right panels), in endometrial carcinoma (A), melanoma (B), and head and neck squamous cell carcinoma (C). The log-rank test was used for survival analyses, and the Wilcoxon two-tailed test was used for the CD8 T-cell association analyses. FIG. 9D-FIG. 9F depict box-plot comparisons of CD8 T-cell infiltration scores in high vs. low FMRP Sub-signature 1 scored endometrial carcinoma (D), melanoma (E), and head and neck squamous cell carcinoma (F) tumor samples. Only up-regulated genes in the FMRP Sub-signature 1 were used for deriving the signature score in this analysis. FIG. 9G-FIG. 91 show distributions of FMRP Pan-signature scores across different tumors grades in endometrial carcinoma (G), melanoma (H), and head and neck squamous cell carcinoma (I). FIG. 9J shows the FMRP-activity score (Pan-Signature) in human breast cancer. i: Box-plot comparison of CD8 T-cell infiltration score in high vs. low FMRP signature scored tumor samples. ii: Box-pot comparison FMRP signature scores in immune-excluded vs. inflamed breast cancer tumors (cohort: GSE177043). iii: Box-pot comparison FMRP signature scores in low vs. high TCR diversity breast cancer tumors (cohort: GSE177043). Wilcoxon two-tailed test. FIG. 9K depicts distributions of FMRP Pan-signature scores across different tumors grades in The TCGA breast cancer cohort. FIG. 9L depicts box-pot comparison FMRP Sub-signature 1 scores in immune-excluded vs. inflamed breast cancer tumors (cohort: GSE177043).

FIG. 10A through FIG. 10H depict the level of tumor inflammation with CD8 T-cell based on the Pan-Signature and in specific cancer cells. FIG. 10A shows anti-correlation of the FMRP Pan-Immuno-suppressive signature score with a CD8 T-cell infiltration signature, estimated by the xCell package, in human pan-cancers. The linear regression model with tumor type as covariate was used to estimate the significance of correlation. FIG. 10B-FIG. 10H show an inverse association of the FMRP Pan-Immunosuppressive signature score with the CD8 T-cell infiltration signature in cancer specific analyses; bladder carcinoma (B), colorectal adenocarcinoma (C), glioma (D), liver carcinoma (E), none-small cell line cancer (F), pancreatic adenocarcinoma (G), thymic epithelial tumor (H). Wilcoxon two-tailed test was used for estimation of significance.

### DETAILED DESCRIPTION

The invention is based on analysis of the gene expression signature induced by fragile X mental retardation protein (FMRP) protein activity in tumors. FMRP protein is broadly upregulated across different types of human cancer and, as shown herein, its functional activity mediates immuno-suppressive effects in the tumor microenvironment, reflected in the pathway activity signatures. The present invention relates to methods for evaluating the downstream signaling activity of FMRP protein in tumors, and thereby predicting prognosis, namely overall survival and progression-free survival of cancer patients, and methods for classifying and stratifying such patients. Moreover, this invention relates to a companion diagnostic that could be used in clinic to stratify and prioritize cancer patients for cancer therapy. Concordant differential expression of genes within the signature lists convey a FMRP pathway activity score disclosed herein that can be used to stratify cancer patients into groups that may differently benefit from the aforementioned and potentially other therapeutic modalities for cancer patients, including drugs that inhibit the functional activity of FMRP.

As used herein, the term “FMRP pathway activity” is also referred to as “FMRP downstream transcriptional network in cancer”, “FMRP cancer network signature score”, “FMRP cancer signature score”, or as “FMRP network activity”.

The present invention identifies molecular gene expression biomarkers that can be used to reveal FMRP functional activity in tumors, and thus stratify cancer patients into groups with high, medium, and low FMRP pathway activity. These biomarkers can associate FMRP pathway activity with overall survival (OS) and progression-free survival (PFS), and response to different form of therapies.

The present invention allows for the stratification of cancer patients based on the level of tumor inflammation and immune-cell infiltration.

### Pan-Signature

In one embodiment, the invention provides a “pan-cancer” gene signature, referred to herein as Pan-Signature. Pan-Signature can be used for developing a gene expression signature score that can be used to evaluate the level of FMRP activity in tumors.

Pan-Signature is an overarching signature list comprising the full panel of biomarker genes (156 genes in total) discovered by comparing FMRP pathway-active vs. FMRP pathway-inactive tumors and cultured cancer cells. This signature reveals the combined effect of FMRP activity in cancer cells as well as within the tumor microenvironment. Pan-Signature is disclosed in Table 1.

TABLE 1

| Official | | Secreted | Up/Down-regulation |
|---|---|---|---|
| Symbol | ensembl_gene_id | proteins | by FMRP |
| EIF4G3 | ENSG00000075151 | | Down-reg |
| SMPDL3B | ENSG00000130768 | | Down-reg |
| VANGL2 | ENSG00000162738 | | Down-reg |
| GBP2 | ENSG00000162645 | | Down-reg |
| POGK | ENSG00000143157 | | Down-reg |
| IFITM2 | ENSG00000185201 | | Down-reg |
| IFITM1 | ENSG00000185885 | | Down-reg |
| IFITM3 | ENSG00000142089 | | Down-reg |
| PDLIM1 | ENSG00000107438 | | Down-reg |
| PRDX5 | ENSG00000126432 | | Down-reg |
| PFKP | ENSG00000067057 | | Down-reg |
| SIPA1L2 | ENSG00000116991 | | Down-reg |
| ACSL5 | ENSG00000197142 | | Down-reg |
| RBP4 | ENSG00000138207 | Secretome | Down-reg |
| BNC1 | ENSG00000169594 | | Down-reg |
| PSME2 | ENSG00000100911 | | Down-reg |
| B2M | ENSG00000166710 | Secretome | Down-reg |
| GAS6 | ENSG00000183087 | Secretome | Down-reg |
| PSME1 | ENSG00000092010 | | Down-reg |
| CKMT1B | ENSG00000237289 | | Down-reg |
| CKMT1A | ENSG00000223572 | | Down-reg |
| WDR89 | ENSG00000140006 | | Down-reg |
| USP50 | ENSG00000170236 | | Down-reg |
| CRIP1 | ENSG00000213145 | | Down-reg |
| CHCHD10 | ENSG00000250479 | | Down-reg |
| ZNF23 | ENSG00000167377 | | Down-reg |
| APOB | ENSG00000084674 | Secretome | Down-reg |
| UBA52 | ENSG00000221983 | | Down-reg |
| POGLUT1 | ENSG00000163389 | | Down-reg |
| PLAC8 | ENSG00000145287 | | Down-reg |
| STAT1 | ENSG00000115415 | | Down-reg |
| PDE5A | ENSG00000138735 | | Down-reg |
| CPEB2 | ENSG00000137449 | | Down-reg |
| PCDHB11 | ENSG00000197479 | | Down-reg |
| PCDHB12 | ENSG00000120328 | | Down-reg |
| PCDHB15 | ENSG00000113248 | Secretome | Down-reg |
| ATP13A4 | ENSG00000127249 | | Down-reg |
| HMGB2 | ENSG00000164104 | Secretome | Down-reg |
| RPL29 | ENSG00000162244 | | Down-reg |
| PPARGC1A | ENSG00000109819 | | Down-reg |
| CHN1 | ENSG00000128656 | | Down-reg |
| CCL8 | ENSG00000108700 | Secretome | Down-reg |
| SLC4A4 | ENSG00000080493 | | Down-reg |
| LSM4 | ENSG00000130520 | | Down-reg |
| KIAA0513 | ENSG00000135709 | | Down-reg |
| NME1 | ENSG00000239672 | | Down-reg |
| BST2 | ENSG00000130303 | | Down-reg |
| TMEM144 | ENSG00000164124 | | Down-reg |
| COL3A1 | ENSG00000168542 | Secretome | Down-reg |
| PSMB10 | ENSG00000205220 | | Down-reg |
| MB21D2 | ENSG00000180611 | | Down-reg |
| ZDHHC23 | ENSG00000184307 | | Down-reg |
| MT2A | ENSG00000125148 | | Down-reg |
| TFAP2A | ENSG00000137203 | | Down-reg |
| PARP12 | ENSG00000059378 | | Down-reg |
| HSPB1 | ENSG00000106211 | | Down-reg |
| HNRNPA2B1 | ENSG00000122566 | | Down-reg |
| ENTPD2 | ENSG00000054179 | | Down-reg |
| MYLIP | ENSG00000007944 | | Down-reg |
| MTMR7 | ENSG00000003987 | | Down-reg |
| PSMB8 | ENSG00000204264 | | Down-reg |
| AUTS2 | ENSG00000158321 | | Down-reg |
| UPP1 | ENSG00000183696 | | Down-reg |
| TAPBP | ENSG00000231925 | | Down-reg |
| KLRG2 | ENSG00000188883 | | Down-reg |
| PSMB9 | ENSG00000240065 | | Down-reg |
| MARCKSL1 | ENSG00000175130 | | Up-reg |
| ID3 | ENSG00000117318 | | Up-reg |
| S100A16 | ENSG00000188643 | | Up-reg |
| PLPP3 | ENSG00000162407 | | Up-reg |
| GADD45A | ENSG00000116717 | | Up-reg |
| S100A4 | ENSG00000196154 | | Up-reg |
| DDAH1 | ENSG00000153904 | | Up-reg |
| MYCL | ENSG00000116990 | | Up-reg |
| CD81 | ENSG00000110651 | | Up-reg |
| SHANK2 | ENSG00000162105 | | Up-reg |
| ITIH2 | ENSG00000151655 | | Up-reg |
| PIK3AP1 | ENSG00000155629 | | Up-reg |
| LHFPL6 | ENSG00000183722 | | Up-reg |
| LGALS3 | ENSG00000131981 | Secretome | Up-reg |
| FRMD5 | ENSG00000171877 | | Up-reg |
| CLDN6 | ENSG00000184697 | | Up-reg |
| TNFRSF12A | ENSG00000006327 | | Up-reg |
| NPC2 | ENSG00000119655 | Secretome | Up-reg |
| CD9 | ENSG00000010278 | | Up-reg |
| ATP11A | ENSG00000068650 | | Up-reg |
| SLC25A21 | ENSG00000183032 | | Up-reg |
| CD63 | ENSG00000135404 | | Up-reg |
| B4GALNT3 | ENSG00000139044 | | Up-reg |
| EMP1 | ENSG00000134531 | | Up-reg |
| CSTB | ENSG00000160213 | | Up-reg |
| WNT10A | ENSG00000135925 | Secretome | Up-reg |
| H3-3B | ENSG00000132475 | | Up-reg |
| RABAC1 | ENSG00000105404 | | Up-reg |
| KCTD17 | ENSG00000100379 | | Up-reg |
| BCAM | ENSG00000187244 | | Up-reg |
| CCL15-CCL14 | ENSG00000275688 | | Up-reg |
| CCL15 | ENSG00000275718 | Secretome | Up-reg |
| CCL23 | ENSG00000274736 | Secretome | Up-reg |
| DLG4 | ENSG00000132535 | | Up-reg |
| SPTSSB | ENSG00000196542 | | Up-reg |
| ANXA5 | ENSG00000164111 | | Up-reg |
| VAPA | ENSG00000101558 | | Up-reg |
| SOGA1 | ENSG00000149639 | | Up-reg |
| CST3 | ENSG00000101439 | Secretome | Up-reg |
| MAP1LC3A | ENSG00000101460 | | Up-reg |
| MAP9 | ENSG00000164114 | | Up-reg |
| LGALS1 | ENSG00000100097 | | Up-reg |
| CCDC149 | ENSG00000181982 | | Up-reg |
| GNAS | ENSG00000087460 | | Up-reg |
| CMBL | ENSG00000164237 | | Up-reg |
| PTPRN | ENSG00000054356 | | Up-reg |
| WTIP | ENSG00000142279 | | Up-reg |
| SPP1 | ENSG00000118785 | Secretome | Up-reg |
| FXR1 | ENSG00000114416 | | Up-reg |
| ARHGEF26 | ENSG00000114790 | | Up-reg |
| PROS1 | ENSG00000184500 | Secretome | Up-reg |
| PARP8 | ENSG00000151883 | | Up-reg |
| EIF4A2 | ENSG00000156976 | | Up-reg |
| OSR1 | ENSG00000143867 | | Up-reg |
| TFF2 | ENSG00000160181 | Secretome | Up-reg |
| ATF4 | ENSG00000128272 | | Up-reg |
| CTSZ | ENSG00000101160 | | Up-reg |
| UCHL1 | ENSG00000154277 | | Up-reg |
| ONECUT2 | ENSG00000119547 | | Up-reg |
| EIF1 | ENSG00000173812 | | Up-reg |
| LAMP2 | ENSG00000005893 | | Up-reg |
| CALD1 | ENSG00000122786 | | Up-reg |
| ATP6V1G1 | ENSG00000136888 | | Up-reg |
| PRSS35 | ENSG00000146250 | Secretome | Up-reg |
| KCNK5 | ENSG00000164626 | | Up-reg |
| CDKN2B | ENSG00000147883 | | Up-reg |
| AEBP1 | ENSG00000106624 | | Up-reg |
| SP8 | ENSG00000164651 | | Up-reg |
| CFTR | ENSG00000001626 | | Up-reg |
| TSPAN7 | ENSG00000156298 | | Up-reg |
| MPP6 | ENSG00000105926 | | Up-reg |
| CYSLTR1 | ENSG00000173198 | | Up-reg |
| FSCN1 | ENSG00000075618 | | Up-reg |
| IL33 | ENSG00000137033 | Secretome | Up-reg |
| PLP2 | ENSG00000102007 | | Up-reg |
| ELFN1 | ENSG00000225968 | | Up-reg |
| IGFBP3 | ENSG00000146674 | | Up-reg |
| SAT1 | ENSG00000130066 | | Up-reg |
| AFAP1L1 | ENSG00000157510 | | Up-reg |
| LPAR4 | ENSG00000147145 | | Up-reg |
| ATP6V1F | ENSG00000128524 | | Up-reg |
| GRINA | ENSG00000178719 | | Up-reg |
| CASD1 | ENSG00000127995 | | Up-reg |
| HS6ST2 | ENSG00000171004 | | Up-reg |
| CD109 | ENSG00000156535 | | Up-reg |
| PGRMC1 | ENSG00000101856 | | Up-reg |
| MAL2 | ENSG00000147676 | | Up-reg |
| PHF19 | ENSG00000119403 | | Up-reg |
| TIMP1 | ENSG00000102265 | Secretome | Up-reg |
| ASAP1 | ENSG00000153317 | | Up-reg |

* Secretome refers to the set of proteins that are differentially secreted by cancer cells with high or low FMRP pathway activity that can for example be used as biomarkers in liquid biopsy assays and other diagnostic bioassays.
“Up-reg indicates that a gene is positively regulated by FMRP-activity and Down-reg conversely indicates that a gene is negatively regulated by FMRP-activity.”

As used herein, EIF4G3: eukaryotic translation initiation factor 4 gamma 3; SMPDL3B: sphingomyelin phosphodiesterase acid like 3B; VANGL2: VANGL planar cell polarity protein 2; GBP2: guanylate binding protein 2; POGK: pogo transposable element derived with KRAB domain; IFITM2: interferon induced transmembrane protein 2; IFITM1: interferon induced transmembrane protein 1; IFITM3: interferon induced transmembrane protein 3; PDLIM1: PDZ and LIM domain 1; PRDX5: peroxiredoxin 5; PFKP: phosphofructokinase, platelet; SIPA1L2: signal induced proliferation associated 1 like 2; ACSL5: acyl-CoA synthetase long chain family member 5; RBP4: retinol binding protein 4; BNC1: basonuclin 1; PSME2: proteasome activator subunit 2; B2M: beta-2-microglobulin; GAS6: growth arrest specific 6; PSME1: proteasome activator subunit 1; CKMT1B: creatine kinase, mitochondrial 1B; CKMT1A: creatine kinase, mitochondrial 1A; WDR89: WD repeat domain 89; USP50: ubiquitin specific peptidase 50; CRIP1: cysteine rich protein 1; CHCHD10: coiled-coil-helix-coiled-coil-helix domain containing 10; ZNF23: zinc finger protein 23; APOB: apolipoprotein B; UBA52: ubiquitin A-52 residue ribosomal protein fusion product 1; POGLUT1: protein 0-glucosyltransferase 1; PLACE: placenta associated 8; STAT1: signal transducer and activator of transcription 1; PDESA: phosphodiesterase 5A; CPEB2: cytoplasmic polyadenylation element binding protein 2; PCDHB11: protocadherin beta 11; PCDHB12: protocadherin beta 12; PCDHB15: protocadherin beta 15; ATP13A4: ATPase 13A4; HMGB2: high mobility group box 2; RPL29: ribosomal protein L29; PPARGC1A: PPARG coactivator 1 alpha; CHN1: chimerin 1; CCL8: C-C motif chemokine ligand 8; SLC4A4: solute carrier family 4 member 4; LSM4: LSM4 homolog, U6 small nuclear RNA and mRNA degradation associated; KIAA0513: KIAA0513; NME1: NME/NM23 nucleoside diphosphate kinase 1; BST2: bone marrow stromal cell antigen 2; TMEM144: transmembrane protein 144; COL3A1: collagen type III alpha 1 chain; PSMB10: proteasome 20S subunit beta 10; MB21D2: Mab-21 domain containing 2; ZDHHC23: zinc finger DHHC-type palmitoyltransferase 23; MT2A: metallothionein 2A; TFAP2A: transcription factor AP-2 alpha; PARP12: poly(ADP-ribose) polymerase family member 12; HSPB1: heat shock protein family B (small) member 1; HNRNPA2B1: heterogeneous nuclear ribonucleoprotein A2/B1; ENTPD2: ectonucleoside triphosphate diphosphohydrolase 2; MYLIP: myosin regulatory light chain interacting protein; MTMR7: myotubularin related protein 7; PSMB8: proteasome 20S subunit beta 8; AUTS2: activator of transcription and developmental regulator AUTS2; UPP1: uridine phosphorylase 1; TAPBP: TAP binding protein; KLRG2: killer cell lectin like receptor G2; PSMB9: proteasome 20S subunit beta 9; MARCKSL1: MARCKS like 1; ID3: inhibitor of DNA binding 3, HLH protein; S100A16: S100 calcium binding protein A16; PLPP3: phospholipid phosphatase 3; GADD45A: growth arrest and DNA damage inducible alpha; S100A4: S100 calcium binding protein A4; DDAHl: dimethylarginine dimethylaminohydrolase 1; MYCL: MYCL proto-oncogene, bHLH transcription factor; CD81: CD81 molecule; SHANK2: SH3 and multiple ankyrin repeat domains; ITIH2: inter-alpha-trypsin inhibitor heavy chain 2; PIK3AP1: phosphoinositide-3-kinase adaptor protein 1; LHFPL6: LHFPL tetraspan subfamily member 6; LGALS3: galectin 3; FRMD5: FERM domain containing 5; CLDN6: claudin 6; TNFRSF12A: TNF receptor superfamily member 12A; NPC2: NPC intracellular cholesterol transporter 2; CD9: CD9 molecule; ATP11A: ATPase phospholipid transporting 11A; SLC25A21: solute carrier family 25 member 21; CD63: CD63 molecule; B4GALNT3: beta-1,4-N-acetyl-galactosaminyltransferase 3; EMPl: epithelial membrane protein 1; CSTB: cystatin B; WNT10A: Wnt family member 10A; H3-3B: H3.3 histone B; RABAC1: Rab acceptor 1; KCTD17: potassium channel tetramerization domain containing 17; BCAM: basal cell adhesion molecule (Lutheran blood group); CCL15-CCL14: CCL15-CCL14 readthrough (NMD candidate); CCL15: C-C motif chemokine ligand 15; CCL23: C-C motif chemokine ligand 23; DLG4: discs large MAGUK scaffold protein 4; SPTSSB: serine palmitoyltransferase small subunit B; ANXAS: annexin A5; VAPA: VAMP associated protein A; SOGA1: suppressor of glucose, autophagy associated 1; CST3: cystatin C; MAP1LC3A: microtubule associated protein 1 light chain 3 alpha; MAPS: microtubule associated protein 9; LGALS1: galectin 1; CCDC149: coiled-coil domain containing 149; GNAS: GNAS complex locus; CMBL: carboxymethylenebutenolidase homolog; PTPRN: protein tyrosine phosphatase receptor type N; WTIP: WT1 interacting protein; SPP1: secreted phosphoprotein 1; FXR1: FMR1 autosomal homolog 1; ARHGEF26: Rho guanine nucleotide exchange factor 26; PROS1: protein S; PARP8: poly(ADP-ribose) polymerase family member 8; EIF4A2: eukaryotic translation initiation factor 4A2; OSR1: odd-skipped related transcription factor 1; TFF2: trefoil factor 2; ATF4: activating transcription factor 4; CTSZ: cathepsin Z; UCHL1: ubiquitin C-terminal hydrolase L1; ONECUT2: one cut homeobox 2; EIF1: eukaryotic translation initiation factor 1; LAMP2: lysosomal associated membrane protein 2; CALD1: caldesmon 1; ATP6V1G1: ATPase H+ transporting V1 subunit G1; PRSS35: serine protease 35; KCNK5: potassium two pore domain channel subfamily K member 5; CDKN2B: cyclin dependent kinase inhibitor 2B; AEBP1: AE binding protein 1; SP8: Sp8 transcription factor; CFTR: CF transmembrane conductance regulator; TSPAN7: tetraspanin 7; MPP6: protein associated with LINT 2, MAGUK family member; CYSLTR1: cysteinyl leukotriene receptor 1; FSCN1: fascin actin-bundling protein 1; IL33: interleukin 33; PLP2: proteolipid protein 22; ELFN1: extracellular leucine rich repeat and fibronectin type III domain containing 1; IGFBP3: insulin like growth factor binding protein 3; SAT1: spermidine/spermine N1-acetyltransferase 1; AFAP1L1: actin filament associated protein 1 like 1; LPAR4: lysophosphatidic acid receptor 4; ATP6V1F: ATPase H+transporting V1 subunit F; GRINA: glutamate ionotropic receptor NMDA type subunit associated protein 1; CASD1: CAS1 domain containing 1; HS6ST2: heparan sulfate 6-O-sulfotransferase 2; CD109: CD109 molecule; PGRMC1: progesterone receptor membrane component 1; MAL2: mal, T cell differentiation protein 2; PHF19 PHD: finger protein 19; TIMP1: TIMP metallopeptidase inhibitor 1; ASAP1: ArfGAP with SH3 domain, ankyrin repeat and PH domain 1.

In notable contrast to the non-association of FMR1 mRNA itself, the FMRP-activity signature revealed a statistically significant association with overall and progression-free survival, such that patients with higher FMRP-activity have worse overall survival and progression-free survival. Moreover, a high FMRP-activity score demonstrates a statistically significant anti-correlation with the CD8 T-cell signature that is diagnostic of CTL abundance in human tumors.

Pan-Signature is, alone, generally sufficient for predicting prognosis, namely overall survival and progression-free survival of cancer patients, and for use in methods for classifying and stratifying such patients; for example, as responders or non-responders to a particular cancer therapy. However, should a diagnostic assay based on Pan-Signature produce non-conclusive results or, additionally/alternatively, if further optimized/more-precise results are desired, the invention further provides 3 sub-signatures and 28 cancer specific signatures, which are described below. All of these subset signatures contain genes that are either up- or down-regulated by FMRP activity as disclosed in the Pan-Signature. Notably, using only up- or-down-regulated genes as a secondary sub-signature of particular signature of sub-signature can have utility on its own, as will be further discussed herein.

### Sub-Signature 1

In one embodiment, the invention provides a “pan-cancer” gene expression signature, referred to herein as Sub-Signature 1. Sub-Signature 1 is a subset of Pan-Signature and is based on genes whose expression defines FMRP pathway activity vs. inactivity in cancer cells, without the effects of stromal and immune cells of the tumor microenvironment (TME). As the result, this signature evaluates the activity of FMRP in cancer cells alone without the effect of TME. Sub-Signature 1 is disclosed in Table 2.

TABLE 2

| Official Symbol | Up/Down-regulation by FMRP |
|---|---|
| EIF4G3 | Down-reg |
| SMPDL3B | Down-reg |
| VANGL2 | Down-reg |
| POGK | Down-reg |
| PDLIM1 | Down-reg |
| PFKP | Down-reg |
| SIPA1L2 | Down-reg |
| BNC1 | Down-reg |
| GAS6 | Down-reg |
| CKMT1B | Down-reg |
| CKMT1A | Down-reg |
| CRIP1 | Down-reg |
| CHCHD10 | Down-reg |
| ZNF23 | Down-reg |
| POGLUT1 | Down-reg |
| PDE5A | Down-reg |
| CPEB2 | Down-reg |
| PCDHB11 | Down-reg |
| PCDHB12 | Down-reg |
| PCDHB15 | Down-reg |
| ATP13A4 | Down-reg |
| PPARGC1A | Down-reg |
| CHN1 | Down-reg |
| SLC4A4 | Down-reg |
| KIAA0513 | Down-reg |
| TMEM144 | Down-reg |
| MB21D2 | Down-reg |
| ZDHHC23 | Down-reg |
| TFAP2A | Down-reg |
| PARP12 | Down-reg |
| ENTPD2 | Down-reg |
| MYLIP | Down-reg |
| MTMR7 | Down-reg |
| AUTS2 | Down-reg |
| UPP1 | Down-reg |
| KLRG2 | Down-reg |
| MARCKSL1 | Up-reg |
| S100A16 | Up-reg |
| DDAH1 | Up-reg |
| MYCL | Up-reg |
| SHANK2 | Up-reg |
| ITIH2 | Up-reg |
| PIK3AP1 | Up-reg |
| LHFPL6 | Up-reg |
| FRMD5 | Up-reg |
| CLDN6 | Up-reg |
| ATP11A | Up-reg |
| SLC25A21 | Up-reg |
| B4GALNT3 | Up-reg |
| WNT10A | Up-reg |
| KCTD17 | Up-reg |
| BCAM | Up-reg |
| CCL15-CCL14 | Up-reg |
| CCL15 | Up-reg |
| CCL23 | Up-reg |
| DLG4 | Up-reg |
| SPTSSB | Up-reg |
| SOGA1 | Up-reg |
| MAP9 | Up-reg |
| CCDC149 | Up-reg |
| CMBL | Up-reg |
| PTPRN | Up-reg |
| WTIP | Up-reg |
| FXR1 | Up-reg |
| ARHGEF26 | Up-reg |
| PROS1 | Up-reg |
| PARP8 | Up-reg |
| OSR1 | Up-reg |
| TFF2 | Up-reg |
| UCHL1 | Up-reg |
| PRSS35 | Up-reg |
| KCNK5 | Up-reg |
| AEBP1 | Up-reg |
| SP8 | Up-reg |
| CFTR | Up-reg |
| CYSLTR1 | Up-reg |
| FSCN1 | Up-reg |
| IL33 | Up-reg |
| ELFN1 | Up-reg |
| AFAP1L1 | Up-reg |
| LPAR4 | Up-reg |
| CASD1 | Up-reg |
| HS6ST2 | Up-reg |
| CD109 | Up-reg |
| MAL2 | Up-reg |
| PHF19 | Up-reg |

“Up-reg indicates that a gene is positively regulated by FMRP-activity and Down-reg conversely indicates that a gene is negatively regulated by FMRP-activity.”

### Sub-Signature 2

In another embodiment, the invention provides a “pan-cancer” gene expression signature, referred to herein as Sub-Signature 2. Sub-Signature 2 is a subset of Pan-Signature and is based solely on genes whose expression defines FMRP pathway activity vs. inactivity in tumors. Therefore, this signature assesses the changes in whole tumors, including the constituent accessory (stromal) and immune cells, as instructed by FMRP activity in the cancer cells, resulting from the effect of cell-cell communication between the cancer cells and other cell types in the tumor micro-environment (TME).” Sub-Signature 2 is disclosed in Table 3.

TABLE 3

| Official Symbol | Up/Down-regulation by FMRP |
|---|---|
| CRIP1 | Down-reg |
| GBP2 | Down-reg |
| IFITM2 | Down-reg |
| IFITM1 | Down-reg |
| IFITM3 | Down-reg |
| PRDX5 | Down-reg |
| ACSL5 | Down-reg |
| RBP4 | Down-reg |
| PSME2 | Down-reg |
| B2M | Down-reg |
| PSME1 | Down-reg |
| WDR89 | Down-reg |
| USP50 | Down-reg |
| APOB | Down-reg |
| UBA52 | Down-reg |
| PLAC8 | Down-reg |
| STAT1 | Down-reg |
| HMGB2 | Down-reg |
| RPL29 | Down-reg |
| CCL8 | Down-reg |
| LSM4 | Down-reg |
| NME1 | Down-reg |
| BST2 | Down-reg |
| COL3A1 | Down-reg |
| PSMB10 | Down-reg |
| MT2A | Down-reg |
| HSPB1 | Down-reg |
| HNRNPA2B1 | Down-reg |
| PSMB8 | Down-reg |
| TAPBP | Down-reg |
| PSMB9 | Down-reg |
| ID3 | Up-reg |
| PLPP3 | Up-reg |
| GADD45A | Up-reg |
| S100A4 | Up-reg |
| CD81 | Up-reg |
| LGALS3 | Up-reg |
| TNFRSF12A | Up-reg |
| NPC2 | Up-reg |
| CD9 | Up-reg |
| CD63 | Up-reg |
| EMP1 | Up-reg |
| CSTB | Up-reg |
| H3-3B | Up-reg |
| RABAC1 | Up-reg |
| ANXA5 | Up-reg |
| VAPA | Up-reg |
| CST3 | Up-reg |
| MAP1LC3A | Up-reg |
| LGALS1 | Up-reg |
| GNAS | Up-reg |
| SPP1 | Up-reg |
| EIF4A2 | Up-reg |
| ATF4 | Up-reg |
| CTSZ | Up-reg |
| ONECUT2 | Up-reg |
| EIF1 | Up-reg |
| LAMP2 | Up-reg |
| CALD1 | Up-reg |
| ATP6V1G1 | Up-reg |
| CDKN2B | Up-reg |
| TSPAN7 | Up-reg |
| MPP6 | Up-reg |
| PLP2 | Up-reg |
| IGFBP3 | Up-reg |
| SAT1 | Up-reg |
| ATP6V1F | Up-reg |
| GRINA | Up-reg |
| PGRMC1 | Up-reg |
| TIMP1 | Up-reg |
| ASAP1 | Up-reg |
| FXR1 | Up-reg |

“Up-reg indicates that a gene is positively regulated by FMRP-activity and Down-reg conversely indicates that a gene is negatively regulated by FMRP-activity.”

### Sub-Signature 3

In another embodiment, the invention provides a “pan-cancer” gene expression signature, referred to herein as Sub-Signature 3. Sub-Signature 3 is a subset of Pan-Signature in which the genes corresponding to the immune response are excluded. Therefore, it can be applied to evaluate FMRP pathway activity without the indirect effects of immune cells in the tumor microenvironment (TME). Sub-Signature 3 is disclosed in Table 4.

TABLE 4

| Official Symbol | Up/Down-regulation by FMRP |
|---|---|
| PRDX5 | Down-reg |
| ACSL5 | Down-reg |
| RBP4 | Down-reg |
| WDR89 | Down-reg |
| USP50 | Down-reg |
| CRIP1 | Down-reg |
| APOB | Down-reg |
| UBA52 | Down-reg |
| PLAC8 | Down-reg |
| RPL29 | Down-reg |
| LSM4 | Down-reg |
| NME1 | Down-reg |
| COL3A1 | Down-reg |
| HSPB1 | Down-reg |
| HNRNPA2B1 | Down-reg |
| TAPBP | Down-reg |
| ID3 | Up-reg |
| PLPP3 | Up-reg |
| GADD45A | Up-reg |
| S100A4 | Up-reg |
| CD81 | Up-reg |
| TNFRSF12A | Up-reg |
| NPC2 | Up-reg |
| CD9 | Up-reg |
| CD63 | Up-reg |
| EMP1 | Up-reg |
| CSTB | Up-reg |
| H3-3B | Up-reg |
| RABAC1 | Up-reg |
| ANXA5 | Up-reg |
| VAPA | Up-reg |
| CST3 | Up-reg |
| MAP1LC3A | Up-reg |
| LGALS1 | Up-reg |
| GNAS | Up-reg |
| SPP1 | Up-reg |
| FXR1 | Up-reg |
| EIF4A2 | Up-reg |
| ATF4 | Up-reg |
| CTSZ | Up-reg |
| ONECUT2 | Up-reg |
| EIF1 | Up-reg |
| LAMP2 | Up-reg |
| CALD1 | Up-reg |
| ATP6V1G1 | Up-reg |
| CDKN2B | Up-reg |
| TSPAN7 | Up-reg |
| MPP6 | Up-reg |
| PLP2 | Up-reg |
| IGFBP3 | Up-reg |
| SAT1 | Up-reg |
| ATP6V1F | Up-reg |
| GRINA | Up-reg |
| PGRMC1 | Up-reg |
| TIMP1 | Up-reg |
| ASAP1 | Up-reg |

“Up-reg” indicates that a gene is positively regulated by FMRP-activity and “Down-reg” conversely indicates that a gene is negatively regulated by FMRP-activity.”

In addition to the four (4) pan-cancer signatures, the invention provides illustrative cancer-specific signatures which have been optimized to selectively score FMRP pathway activity in 28 individual cancer types. The 28 cancer specific signatures are shown in Tables 5-32 below.

| | | Table 6 - | | | |
|---|---|---|---|---|---|
| | | Lung | Table 7 - | | |
| | Table 5 - | squamous | Hepato- | Table 8 - | Table 9 - |
| Official | Lung | cell | cellular | Pancreatic | Prostate |
| Symbol | Adenocarcinoma | carcinoma | carcinoma | adenocarcinoma | adenocarcinoma |
| EIF4G3 | 0 | 0 | 0 | 0 | 0 |
| SMPDL3B | Down-reg | 0 | 0 | 0 | Down-reg |
| VANGL2 | 0 | Down-reg | Down-reg | 0 | Down-reg |
| GBP2 | 0 | 0 | Down-reg | 0 | Down-reg |
| POGK | Down-reg | 0 | 0 | 0 | 0 |
| IFITM2 | 0 | 0 | Down-reg | 0 | Down-reg |
| IFITM1 | 0 | 0 | Down-reg | 0 | Down-reg |
| IFITM3 | 0 | 0 | Down-reg | 0 | Down-reg |
| PDLIM1 | 0 | 0 | Down-reg | 0 | Down-reg |
| PRDX5 | 0 | 0 | 0 | 0 | Down-reg |
| PFKP | 0 | 0 | 0 | 0 | 0 |
| SIPA1L2 | 0 | Down-reg | 0 | Down-reg | Down-reg |
| ACSL5 | Down-reg | 0 | 0 | 0 | 0 |
| RBP4 | 0 | 0 | Down-reg | Down-reg | 0 |
| BNC1 | 0 | Down-reg | 0 | Down-reg | Down-reg |
| PSME2 | 0 | 0 | 0 | 0 | Down-reg |
| B2M | 0 | Down-reg | Down-reg | 0 | Down-reg |
| GAS6 | Down-reg | 0 | Down-reg | 0 | Down-reg |
| PSME1 | 0 | 0 | Down-reg | 0 | Down-reg |
| CKMT1B | 0 | Down-reg | 0 | Down-reg | Down-reg |
| CKMT1A | 0 | Down-reg | 0 | 0 | 0 |
| WDR89 | 0 | Down-reg | 0 | 0 | 0 |
| USP50 | 0 | 0 | 0 | 0 | 0 |
| CRIP1 | 0 | 0 | Down-reg | 0 | Down-reg |
| CHCHD10 | 0 | Down-reg | Down-reg | Down-reg | Down-reg |
| ZNF23 | Down-reg | 0 | Down-reg | Down-reg | 0 |
| APOB | 0 | 0 | Down-reg | 0 | Down-reg |
| UBA52 | 0 | Down-reg | 0 | 0 | Down-reg |
| POGLUT1 | 0 | 0 | 0 | 0 | 0 |
| PLAC8 | Down-reg | Down-reg | Down-reg | 0 | 0 |
| STAT1 | 0 | 0 | 0 | 0 | Down-reg |
| PDE5A | Down-reg | 0 | Down-reg | Down-reg | Down-reg |
| CPEB2 | 0 | 0 | Down-reg | 0 | Down-reg |
| PCDHB11 | 0 | 0 | Down-reg | Down-reg | 0 |
| PCDHB12 | 0 | 0 | 0 | Down-reg | 0 |
| PCDHB15 | 0 | Down-reg | 0 | Down-reg | Down-reg |
| ATP13A4 | Down-reg | 0 | 0 | 0 | Down-reg |
| HMGB2 | 0 | Down-reg | 0 | 0 | 0 |
| RPL29 | 0 | Down-reg | 0 | 0 | 0 |
| PPARGC1A | 0 | 0 | Down-reg | Down-reg | Down-reg |
| CHN1 | 0 | 0 | Down-reg | Down-reg | 0 |
| CCL8 | 0 | 0 | Down-reg | Down-reg | Down-reg |
| SLC4A4 | Down-reg | 0 | 0 | 0 | Down-reg |
| LSM4 | 0 | Down-reg | 0 | 0 | Down-reg |
| KIAA0513 | Down-reg | 0 | 0 | Down-reg | Down-reg |
| NME1 | 0 | 0 | 0 | 0 | 0 |
| BST2 | Down-reg | 0 | Down-reg | 0 | Down-reg |
| TMEM144 | 0 | 0 | 0 | 0 | Down-reg |
| COL3A1 | 0 | 0 | 0 | O | Down-reg |
| PSMB10 | 0 | 0 | Down-reg | 0 | 0 |
| MB21D2 | 0 | 0 | 0 | 0 | 0 |
| ZDHHC23 | 0 | 0 | 0 | 0 | 0 |
| MT2A | 0 | 0 | Down-reg | 0 | Down-reg |
| TFAP2A | 0 | Down-reg | 0 | 0 | 0 |
| PARP12 | 0 | 0 | 0 | 0 | Down-reg |
| HSPB1 | Down-reg | Down-reg | 0 | 0 | Down-reg |
| HNRNPA2B1 | 0 | Down-reg | 0 | 0 | 0 |
| ENTPD2 | 0 | 0 | 0 | 0 | 0 |
| MYLIP | Down-reg | Down-reg | 0 | Down-reg | 0 |
| MTMR7 | Down-reg | 0 | 0 | Down-reg | 0 |
| PSMB8 | 0 | 0 | Down-reg | 0 | 0 |
| AUTS2 | Down-reg | Down-reg | Down-reg | Down-reg | Down-reg |
| UPP1 | 0 | 0 | 0 | 0 | Down-reg |
| TAPBP | 0 | 0 | Down-reg | 0 | Down-reg |
| KLRG2 | Down-reg | Down-reg | 0 | 0 | 0 |
| PSMB9 | 0 | 0 | 0 | 0 | Down-reg |
| MARCKSL1 | 0 | 0 | Up-reg | 0 | Up-reg |
| ID3 | Up-reg | Up-reg | 0 | 0 | 0 |
| S100A16 | Up-reg | 0 | Up-reg | Up-reg | 0 |
| PLPP3 | 0 | 0 | 0 | 0 | 0 |
| GADD45A | Up-reg | 0 | 0 | Up-reg | 0 |
| S100A4 | 0 | Up-reg | 0 | Up-reg | 0 |
| DDAH1 | 0 | Up-reg | 0 | Up-reg | 0 |
| MYCL | 0 | 0 | 0 | 0 | 0 |
| CD81 | 0 | Up-reg | 0 | 0 | 0 |
| SHANK2 | 0 | Up-reg | 0 | Up-reg | 0 |
| ITIH2 | 0 | Up-reg | 0 | 0 | 0 |
| PIK3AP1 | 0 | 0 | 0 | Up-reg | 0 |
| LHFPL6 | 0 | 0 | 0 | 0 | 0 |
| LGALS3 | Up-reg | 0 | Up-reg | Up-reg | 0 |
| FRMD5 | Up-reg | Up-reg | 0 | Up-reg | Up-reg |
| CLDN6 | Up-reg | Up-reg | 0 | Up-reg | Up-reg |
| TNFRSF12A | Up-reg | Up-reg | Up-reg | Up-reg | Up-reg |
| NPC2 | 0 | Up-reg | Up-reg | Up-reg | 0 |
| CD9 | 0 | 0 | 0 | Up-reg | 0 |
| ATP11A | 0 | Up-reg | Up-reg | Up-reg | 0 |
| SLC25A21 | Up-reg | 0 | Up-reg | Up-reg | Up-reg |
| CD63 | 0 | Up-reg | Up-reg | 0 | Up-reg |
| B4GALNT3 | 0 | Up-reg | Up-reg | Up-reg | Up-reg |
| EMP1 | 0 | 0 | 0 | Up-reg | 0 |
| CSTB | 0 | 0 | Up-reg | Up-reg | 0 |
| WNT10A | 0 | Up-reg | 0 | Up-reg | Up-reg |
| H3-3B | 0 | 0 | 0 | 0 | 0 |
| RABAC1 | 0 | Up-reg | 0 | 0 | Up-reg |
| KCTD17 | 0 | 0 | Up-reg | 0 | Up-reg |
| BCAM | 0 | Up-reg | 0 | 0 | 0 |
| CCL15-CCL14 | 0 | 0 | 0 | 0 | 0 |
| CCL15 | 0 | Up-reg | 0 | 0 | 0 |
| CCL23 | 0 | Up-reg | 0 | 0 | 0 |
| DLG4 | 0 | Up-reg | Up-reg | 0 | Up-reg |
| SPTSSB | 0 | 0 | 0 | 0 | 0 |
| ANXA5 | 0 | Up-reg | Up-reg | Up-reg | 0 |
| VAPA | 0 | Up-reg | 0 | Up-reg | 0 |
| SOGA1 | 0 | 0 | 0 | 0 | 0 |
| CST3 | O | Up-reg | Up-reg | 0 | 0 |
| MAP1LC3A | O | Up-reg | 0 | 0 | Up-reg |
| MAP9 | 0 | Up-reg | 0 | 0 | 0 |
| LGALS1 | Up-reg | Up-reg | Up-reg | Up-reg | 0 |
| CCDC149 | 0 | Up-reg | Up-reg | 0 | Up-reg |
| GNAS | 0 | 0 | 0 | 0 | 0 |
| CMBL | 0 | 0 | 0 | 0 | 0 |
| PTPRN | Up-reg | Up-reg | Up-reg | 0 | 0 |
| WTIP | 0 | Up-reg | 0 | Up-reg | Up-reg |
| SPP1 | 0 | 0 | Up-reg | Up-reg | Up-reg |
| FXR1 | Up-reg | 0 | Up-reg | Up-reg | Up-reg |
| ARHGEF26 | 0 | 0 | 0 | 0 | 0 |
| PROS1 | 0 | Up-reg | 0 | 0 | Up-reg |
| PARP8 | 0 | Up-reg | Up-reg | 0 | 0 |
| EIF4A2 | 0 | 0 | Up-reg | Up-reg | Up-reg |
| OSR1 | 0 | Up-reg | Up-reg | 0 | 0 |
| TFF2 | 0 | 0 | Up-reg | Up-reg | 0 |
| ATF4 | 0 | 0 | Up-reg | 0 | Up-reg |
| CTSZ | 0 | Up-reg | 0 | Up-reg | Up-reg |
| UCHL1 | Up-reg | 0 | Up-reg | 0 | 0 |
| ONECUT2 | 0 | 0 | 0 | Up-reg | Up-reg |
| EIF1 | 0 | 0 | 0 | 0 | 0 |
| LAMP2 | 0 | 0 | 0 | 0 | 0 |
| CALD1 | Up-reg | Up-reg | 0 | Up-reg | 0 |
| ATP6V1G1 | 0 | 0 | 0 | 0 | Up-reg |
| PRSS35 | 0 | Up-reg | Up-reg | 0 | 0 |
| KCNK5 | O | Up-reg | 0 | Up-reg | Up-reg |
| CDKN2B | 0 | 0 | Up-reg | Up-reg | Up-reg |
| AEBP1 | 0 | Up-reg | 0 | Up-reg | Up-reg |
| SP8 | Up-reg | 0 | 0 | 0 | Up-reg |
| CFTR | 0 | Up-reg | Up-reg | Up-reg | 0 |
| TSPAN7 | 0 | 0 | Up-reg | 0 | 0 |
| MPP6 | Up-reg | 0 | Up-reg | Up-reg | Up-reg |
| CYSLTR1 | 0 | Up-reg | 0 | 0 | Up-reg |
| FSCN1 | Up-reg | 0 | Up-reg | Up-reg | Up-reg |
| IL33 | 0 | 0 | 0 | 0 | 0 |
| PLP2 | Up-reg | 0 | Up-reg | Up-reg | 0 |
| ELFN1 | Up-reg | 0 | 0 | 0 | Up-reg |
| IGFBP3 | Up-reg | 0 | Up-reg | Up-reg | Up-reg |
| SAT1 | 0 | 0 | 0 | Up-reg | Up-reg |
| AFAP1L1 | Up-reg | Up-reg | 0 | 0 | 0 |
| LPAR4 | Up-reg | 0 | 0 | 0 | 0 |
| ATP6V1F | 0 | 0 | Up-reg | 0 | Up-reg |
| GRINA | 0 | Up-reg | 0 | 0 | Up-reg |
| CASD1 | 0 | Up-reg | 0 | 0 | 0 |
| HS6ST2 | 0 | 0 | Up-reg | 0 | 0 |
| CD109 | Up-reg | 0 | 0 | Up-reg | 0 |
| PGRMC1 | 0 | 0 | 0 | 0 | Up-reg |
| MAL2 | 0 | 0 | Up-reg | Up-reg | Up-reg |
| PHF19 | 0 | Up-reg | Up-reg | 0 | Up-reg |
| TIMP1 | Up-reg | Up-reg | Up-reg | Up-reg | 0 |
| ASAP1 | Up-reg | Up-reg | Up-reg | Up-reg | 0 |

“Up-reg” indicates that a gene is positively regulated by FMRP-activity and “Down-reg” conversely indicates that a gene is negatively regulated by FMRP-activity, and 0 shows the genes not correlated with or regulated by FMRP-activity for this particular cancer type.

| | Table 10 - | Table 11 - | Table 12 - | Table 13 - | Table 14 - |
|---|---|---|---|---|---|
| Official | Breast | Cervical | Endometrial | Ovarian | Testicular |
| Symbol | Carcinoma | Carcinoma | Carcinoma | Carcinoma | Tumors |
| EIF4G3 | 0 | 0 | 0 | 0 | Down-reg |
| SMPDL3B | Down-reg | 0 | 0 | 0 | Down-reg |
| VANGL2 | Down-reg | 0 | 0 | 0 | Down-reg |
| GBP2 | Down-reg | Down-reg | Down-reg | Down-reg | Down-reg |
| POGK | Down-reg | 0 | 0 | 0 | Down-reg |
| IFITM2 | Down-reg | 0 | Down-reg | 0 | 0 |
| IFITM1 | Down-reg | 0 | 0 | 0 | 0 |
| IFITM3 | Down-reg | 0 | 0 | 0 | 0 |
| PDLIM1 | 0 | Down-reg | Down-reg | Down-reg | Down-reg |
| PRDX5 | Down-reg | 0 | 0 | Down-reg | 0 |
| PFKP | 0 | 0 | 0 | 0 | 0 |
| SIPA1L2 | Down-reg | 0 | Down-reg | 0 | Down-reg |
| ACSL5 | Down-reg | Down-reg | Down-reg | 0 | 0 |
| RBP4 | 0 | 0 | 0 | 0 | 0 |
| BNC1 | 0 | Down-reg | 0 | 0 | Down-reg |
| PSME2 | Down-reg | Down-reg | 0 | Down-reg | Down-reg |
| B2M | Down-reg | Down-reg | Down-reg | 0 | Down-reg |
| GAS6 | 0 | 0 | 0 | 0 | 0 |
| PSME1 | Down-reg | Down-reg | 0 | Down-reg | Down-reg |
| CKMT1B | Down-reg | Down-reg | 0 | 0 | Down-reg |
| CKMT1A | Down-reg | Down-reg | 0 | 0 | 0 |
| WDR89 | Down-reg | 0 | Down-reg | 0 | 0 |
| USP50 | 0 | 0 | 0 | 0 | 0 |
| CRIP1 | Down-reg | Down-reg | Down-reg | 0 | 0 |
| CHCHD10 | 0 | Down-reg | 0 | 0 | 0 |
| ZNF23 | 0 | 0 | Down-reg | 0 | Down-reg |
| APOB | 0 | Down-reg | 0 | 0 | 0 |
| UBA52 | Down-reg | Down-reg | 0 | 0 | Down-reg |
| POGLUT1 | 0 | 0 | 0 | 0 | 0 |
| PLAC8 | Down-reg | Down-reg | Down-reg | 0 | 0 |
| STAT1 | Down-reg | Down-reg | 0 | Down-reg | Down-reg |
| PDE5A | 0 | Down-reg | Down-reg | 0 | 0 |
| CPEB2 | 0 | 0 | Down-reg | 0 | 0 |
| PCDHB11 | 0 | 0 | 0 | 0 | Down-reg |
| PCDHB12 | 0 | 0 | 0 | Down-reg | 0 |
| PCDHB15 | 0 | 0 | Down-reg | Down-reg | Down-reg |
| ATP13A4 | 0 | Down-reg | 0 | 0 | 0 |
| HMGB2 | Down-reg | Down-reg | 0 | 0 | 0 |
| RPL29 | Down-reg | 0 | Down-reg | 0 | Down-reg |
| PPARGC1A | 0 | 0 | 0 | 0 | 0 |
| CHN1 | 0 | 0 | 0 | 0 | Down-reg |
| CCL8 | 0 | 0 | 0 | Down-reg | 0 |
| SLC4A4 | 0 | 0 | 0 | Down-reg | 0 |
| LSM4 | Down-reg | Down-reg | 0 | Down-reg | 0 |
| KIAA0513 | 0 | Down-reg | Down-reg | Down-reg | Down-reg |
| NME1 | Down-reg | 0 | 0 | Down-reg | 0 |
| BST2 | Down-reg | 0 | 0 | Down-reg | 0 |
| TMEM144 | Down-reg | 0 | Down-reg | Down-reg | Down-reg |
| COL3A1 | 0 | 0 | 0 | 0 | 0 |
| PSMB10 | Down-reg | Down-reg | Down-reg | 0 | 0 |
| MB21D2 | 0 | 0 | 0 | 0 | 0 |
| ZDHHC23 | 0 | 0 | 0 | Down-reg | Down-reg |
| MT2A | 0 | 0 | 0 | 0 | 0 |
| TFAP2A | Down-reg | Down-reg | Down-reg | Down-reg | 0 |
| PARP12 | Down-reg | Down-reg | 0 | Down-reg | Down-reg |
| HSPB1 | Down-reg | Down-reg | Down-reg | 0 | Down-reg |
| HNRNPA2B1 | Down-reg | 0 | 0 | Down-reg | Down-reg |
| ENTPD2 | 0 | 0 | Down-reg | Down-reg | 0 |
| MYLIP | Down-reg | Down-reg | Down-reg | 0 | Down-reg |
| MTMR7 | 0 | Down-reg | 0 | 0 | Down-reg |
| PSMB8 | Down-reg | Down-reg | Down-reg | Down-reg | 0 |
| AUTS2 | Down-reg | 0 | 0 | 0 | Down-reg |
| UPP1 | 0 | 0 | 0 | 0 | 0 |
| TAPBP | Down-reg | Down-reg | Down-reg | Down-reg | 0 |
| KLRG2 | 0 | Down-reg | 0 | Down-reg | 0 |
| PSMB9 | Down-reg | Down-reg | Down-reg | Down-reg | 0 |
| MARCKSL1 | 0 | Up-reg | 0 | 0 | Up-reg |
| ID3 | Up-reg | Up-reg | 0 | Up-reg | Up-reg |
| S100A16 | Up-reg | 0 | 0 | 0 | Up-reg |
| PLPP3 | 0 | 0 | 0 | 0 | 0 |
| GADD45A | 0 | Up-reg | 0 | 0 | 0 |
| S100A4 | Up-reg | 0 | Up-reg | 0 | Up-reg |
| DDAH1 | 0 | Up-reg | 0 | 0 | 0 |
| MYCL | 0 | 0 | 0 | 0 | 0 |
| CD81 | Up-reg | Up-reg | 0 | Up-reg | Up-reg |
| SHANK2 | 0 | Up-reg | 0 | 0 | 0 |
| ITIH2 | Up-reg | 0 | 0 | 0 | Up-reg |
| PIK3AP1 | 0 | 0 | 0 | 0 | Up-reg |
| LHFPL6 | 0 | 0 | 0 | 0 | 0 |
| LGALS3 | Up-reg | 0 | 0 | 0 | Up-reg |
| FRMD5 | Up-reg | Up-reg | Up-reg | Up-reg | 0 |
| CLDN6 | Up-reg | Up-reg | Up-reg | 0 | Up-reg |
| TNFRSF12A | 0 | Up-reg | 0 | 0 | Up-reg |
| NPC2 | 0 | 0 | 0 | Up-reg | Up-reg |
| CD9 | 0 | 0 | Up-reg | Up-reg | Up-reg |
| ATP11A | Up-reg | Up-reg | 0 | Up-reg | Up-reg |
| SLC25A21 | 0 | 0 | 0 | 0 | Up-reg |
| CD63 | 0 | Up-reg | 0 | 0 | Up-reg |
| B4GALNT3 | 0 | Up-reg | 0 | 0 | Up-reg |
| EMP1 | Up-reg | Up-reg | Up-reg | Up-reg | Up-reg |
| CSTB | 0 | 0 | 0 | Up-reg | Up-reg |
| WNT10A | 0 | 0 | Up-reg | 0 | Up-reg |
| H3-3B | 0 | 0 | 0 | 0 | 0 |
| RABAC1 | 0 | Up-reg | 0 | Up-reg | Up-reg |
| KCTD17 | 0 | 0 | Up-reg | Up-reg | 0 |
| BCAM | 0 | 0 | Up-reg | 0 | Up-reg |
| CCL15-CCL14 | 0 | 0 | 0 | 0 | 0 |
| CCL15 | 0 | 0 | 0 | 0 | Up-reg |
| CCL23 | 0 | 0 | Up-reg | 0 | 0 |
| DLG4 | 0 | Up-reg | Up-reg | 0 | Up-reg |
| SPTSSB | 0 | 0 | 0 | 0 | 0 |
| ANXA5 | Up-reg | Up-reg | 0 | Up-reg | 0 |
| VAPA | 0 | 0 | Up-reg | 0 | Up-reg |
| SOGA1 | 0 | 0 | 0 | 0 | 0 |
| CST3 | 0 | Up-reg | 0 | 0 | Up-reg |
| MAP1LC3A | 0 | Up-reg | 0 | Up-reg | 0 |
| MAP9 | Up-reg | Up-reg | 0 | 0 | 0 |
| LGALS1 | Up-reg | Up-reg | 0 | Up-reg | Up-reg |
| CCDC149 | Up-reg | 0 | 0 | 0 | 0 |
| GNAS | 0 | Up-reg | 0 | 0 | 0 |
| CMBL | 0 | Up-reg | 0 | 0 | Up-reg |
| PTPRN | Up-reg | Up-reg | Up-reg | Up-reg | Up-reg |
| WTIP | 0 | Up-reg | Up-reg | 0 | Up-reg |
| SPP1 | 0 | Up-reg | Up-reg | Up-reg | Up-reg |
| FXR1 | 0 | Up-reg | Up-reg | Up-reg | 0 |
| ARHGEF26 | 0 | 0 | 0 | 0 | 0 |
| PROS1 | Up-reg | Up-reg | Up-reg | 0 | 0 |
| PARP8 | 0 | Up-reg | 0 | Up-reg | Up-reg |
| EIF4A2 | Up-reg | Up-reg | Up-reg | 0 | Up-reg |
| OSR1 | 0 | Up-reg | 0 | Up-reg | Up-reg |
| TFF2 | 0 | 0 | 0 | 0 | Up-reg |
| ATF4 | Up-reg | 0 | 0 | Up-reg | Up-reg |
| CTSZ | 0 | Up-reg | 0 | 0 | Up-reg |
| UCHL1 | Up-reg | 0 | Up-reg | Up-reg | 0 |
| ONECUT2 | Up-reg | 0 | Up-reg | Up-reg | Up-reg |
| EIF1 | Up-reg | 0 | 0 | 0 | Up-reg |
| LAMP2 | Up-reg | 0 | 0 | Up-reg | Up-reg |
| CALD1 | Up-reg | Up-reg | 0 | Up-reg | Up-reg |
| ATP6V1G1 | 0 | Up-reg | 0 | 0 | Up-reg |
| PRSS35 | Up-reg | Up-reg | Up-reg | 0 | Up-reg |
| KCNK5 | 0 | 0 | Up-reg | 0 | 0 |
| CDKN2B | Up-reg | 0 | Up-reg | 0 | 0 |
| AEBP1 | Up-reg | Up-reg | Up-reg | Up-reg | Up-reg |
| SP8 | 0 | Up-reg | Up-reg | 0 | Up-reg |
| CFTR | 0 | Up-reg | Up-reg | 0 | Up-reg |
| TSPAN7 | 0 | 0 | Up-reg | 0 | Up-reg |
| MPP6 | Up-reg | Up-reg | Up-reg | 0 | 0 |
| CYSLTR1 | Up-reg | 0 | 0 | 0 | Up-reg |
| FSCN1 | Up-reg | Up-reg | Up-reg | Up-reg | Up-reg |
| IL33 | 0 | Up-reg | 0 | Up-reg | 0 |
| PLP2 | 0 | 0 | Up-reg | Up-reg | Up-reg |
| ELFN1 | 0 | Up-reg | 0 | 0 | Up-reg |
| IGFBP3 | Up-reg | Up-reg | 0 | 0 | Up-reg |
| SAT1 | 0 | 0 | 0 | 0 | Up-reg |
| AFAP1L1 | Up-reg | Up-reg | Up-reg | 0 | 0 |
| LPAR4 | Up-reg | Up-reg | Up-reg | Up-reg | 0 |
| ATP6V1F | 0 | 0 | Up-reg | Up-reg | Up-reg |
| GRINA | 0 | Up-reg | Up-reg | 0 | Up-reg |
| CASD1 | 0 | Up-reg | 0 | 0 | Up-reg |
| HS6ST2 | Up-reg | Up-reg | 0 | 0 | Up-reg |
| CD109 | Up-reg | Up-reg | 0 | 0 | Up-reg |
| PGRMC1 | Up-reg | Up-reg | 0 | 0 | Up-reg |
| MAL2 | 0 | 0 | Up-reg | 0 | Up-reg |
| PHF19 | Up-reg | 0 | Up-reg | 0 | Up-reg |
| TIMP1 | 0 | Up-reg | 0 | Up-reg | Up-reg |
| ASAP1 | Up-reg | Up-reg | Up-reg | 0 | 0 |

“Up-reg” indicates that a gene is positively regulated by FMRP-activity and “Down-reg” conversely indicates that a gene is negatively regulated by FMRP-activity, and 0 shows the genes not correlated with or regulated by FMRP-activity for this particular cancer type.

| | Table 15 - | | | | |
|---|---|---|---|---|---|
| | Head and | Table 16 - | Table 17 - | Table 18 - | Table 19 - |
| Official | Neck | Esophageal | Stomach | Colon | Rectal |
| Symbol | carcinoma | carcinoma | adenocarcinoma | adenocarcinoma | adenocarcinoma |
| EIF4G3 | 0 | Down-reg | 0 | Down-reg | 0 |
| SMPDL3B | Down-reg | Down-reg | 0 | Down-reg | Down-reg |
| VANGL2 | 0 | 0 | 0 | 0 | Down-reg |
| GBP2 | 0 | 0 | 0 | Down-reg | Down-reg |
| POGK | 0 | 0 | 0 | 0 | Down-reg |
| IFITM2 | 0 | 0 | 0 | Down-reg | 0 |
| IFITM1 | 0 | 0 | 0 | Down-reg | Down-reg |
| IFITM3 | 0 | 0 | 0 | 0 | Down-reg |
| PDLIM1 | 0 | 0 | 0 | Down-reg | Down-reg |
| PRDX5 | 0 | Down-reg | 0 | 0 | Down-reg |
| PFKP | 0 | Down-reg | Down-reg | 0 | 0 |
| SIPA1L2 | Down-reg | 0 | 0 | Down-reg | 0 |
| ACSL5 | Down-reg | Down-reg | Down-reg | Down-reg | Down-reg |
| RBP4 | 0 | 0 | 0 | 0 | 0 |
| BNC1 | 0 | Down-reg | 0 | 0 | 0 |
| PSME2 | 0 | 0 | Down-reg | Down-reg | Down-reg |
| B2M | 0 | 0 | 0 | 0 | Down-reg |
| GAS6 | 0 | 0 | Down-reg | 0 | 0 |
| PSME1 | 0 | 0 | 0 | Down-reg | Down-reg |
| CKMT1B | 0 | 0 | Down-reg | 0 | Down-reg |
| CKMT1A | 0 | 0 | Down-reg | Down-reg | 0 |
| WDR89 | 0 | 0 | Down-reg | Down-reg | Down-reg |
| USP50 | 0 | 0 | 0 | 0 | 0 |
| CRIP1 | 0 | Down-reg | 0 | 0 | Down-reg |
| CHCHD10 | 0 | Down-reg | Down-reg | 0 | Down-reg |
| ZNF23 | 0 | 0 | 0 | 0 | Down-reg |
| APOB | 0 | Down-reg | 0 | 0 | Down-reg |
| UBA52 | 0 | 0 | 0 | Down-reg | 0 |
| POGLUT1 | 0 | 0 | 0 | 0 | 0 |
| PLAC8 | Down-reg | Down-reg | Down-reg | Down-reg | Down-reg |
| STAT1 | 0 | 0 | 0 | 0 | 0 |
| PDE5A | Down-reg | Down-reg | 0 | 0 | 0 |
| CPEB2 | Down-reg | Down-reg | 0 | Down-reg | 0 |
| PCDHB11 | 0 | 0 | 0 | 0 | 0 |
| PCDHB12 | 0 | 0 | 0 | 0 | 0 |
| PCDHB15 | 0 | Down-reg | 0 | 0 | 0 |
| ATP13A4 | Down-reg | Down-reg | 0 | 0 | 0 |
| HMGB2 | 0 | 0 | Down-reg | Down-reg | Down-reg |
| RPL29 | 0 | 0 | 0 | Down-reg | 0 |
| PPARGC1A | 0 | Down-reg | Down-reg | Down-reg | 0 |
| CHN1 | Down-reg | 0 | 0 | Down-reg | 0 |
| CCL8 | 0 | 0 | Down-reg | 0 | 0 |
| SLC4A4 | 0 | Down-reg | 0 | Down-reg | Down-reg |
| LSM4 | 0 | 0 | 0 | Down-reg | Down-reg |
| KIAA0513 | Down-reg | Down-reg | 0 | Down-reg | 0 |
| NME1 | 0 | 0 | Down-reg | Down-reg | Down-reg |
| BST2 | 0 | 0 | 0 | 0 | Down-reg |
| TMEM144 | 0 | 0 | 0 | Down-reg | Down-reg |
| COL3A1 | 0 | 0 | 0 | 0 | Down-reg |
| PSMB10 | 0 | 0 | Down-reg | Down-reg | 0 |
| MB21D2 | 0 | 0 | 0 | 0 | 0 |
| ZDHHC23 | Down-reg | 0 | 0 | 0 | Down-reg |
| MT2A | 0 | Down-reg | 0 | 0 | 0 |
| TFAP2A | 0 | Down-reg | Down-reg | 0 | Down-reg |
| PARP12 | Down-reg | Down-reg | 0 | 0 | Down-reg |
| HSPB1 | Down-reg | 0 | 0 | 0 | 0 |
| HNRNPA2B1 | 0 | 0 | Down-reg | Down-reg | Down-reg |
| ENTPD2 | 0 | Down-reg | Down-reg | 0 | 0 |
| MYLIP | Down-reg | 0 | 0 | 0 | Down-reg |
| MTMR7 | Down-reg | 0 | 0 | 0 | 0 |
| PSMB8 | 0 | 0 | 0 | Down-reg | Down-reg |
| AUTS2 | Down-reg | Down-reg | 0 | 0 | 0 |
| UPP1 | 0 | 0 | 0 | 0 | 0 |
| TAPBP | 0 | Down-reg | 0 | 0 | 0 |
| KLRG2 | Down-reg | 0 | 0 | 0 | 0 |
| PSMB9 | 0 | 0 | 0 | Down-reg | Down-reg |
| MARCKSL1 | 0 | 0 | 0 | 0 | Up-reg |
| ID3 | 0 | Up-reg | 0 | Up-reg | 0 |
| S100A16 | Up-reg | 0 | 0 | Up-reg | Up-reg |
| PLPP3 | 0 | 0 | 0 | 0 | 0 |
| GADD45A | Up-reg | Up-reg | 0 | Up-reg | Up-reg |
| S100A4 | 0 | Up-reg | 0 | Up-reg | Up-reg |
| DDAH1 | 0 | Up-reg | 0 | 0 | 0 |
| MYCL | 0 | 0 | 0 | 0 | 0 |
| CD81 | Up-reg | 0 | Up-reg | Up-reg | Up-reg |
| SHANK2 | Up-reg | 0 | 0 | Up-reg | 0 |
| ITIH2 | 0 | Up-reg | Up-reg | 0 | 0 |
| PIK3AP1 | 0 | 0 | 0 | 0 | Up-reg |
| LHFPL6 | 0 | 0 | 0 | 0 | 0 |
| LGALS3 | 0 | 0 | 0 | 0 | Up-reg |
| FRMD5 | Up-reg | 0 | 0 | Up-reg | 0 |
| CLDN6 | Up-reg | Up-reg | Up-reg | Up-reg | 0 |
| TNFRSF12A | Up-reg | 0 | Up-reg | 0 | 0 |
| NPC2 | Up-reg | Up-reg | Up-reg | Up-reg | Up-reg |
| CD9 | 0 | Up-reg | 0 | 0 | 0 |
| ATP11A | Up-reg | Up-reg | 0 | Up-reg | Up-reg |
| SLC25A21 | 0 | Up-reg | Up-reg | Up-reg | Up-reg |
| CD63 | 0 | Up-reg | Up-reg | Up-reg | Up-reg |
| B4GALNT3 | 0 | 0 | 0 | 0 | 0 |
| EMP1 | 0 | 0 | Up-reg | Up-reg | 0 |
| CSTB | 0 | Up-reg | 0 | 0 | Up-reg |
| WNT10A | 0 | Up-reg | Up-reg | Up-reg | Up-reg |
| H3-3B | 0 | 0 | 0 | 0 | 0 |
| RABAC1 | Up-reg | 0 | Up-reg | 0 | Up-reg |
| KCTD17 | Up-reg | Up-reg | 0 | Up-reg | 0 |
| BCAM | Up-reg | 0 | Up-reg | Up-reg | Up-reg |
| CCL15-CCL14 | 0 | 0 | 0 | 0 | 0 |
| CCL15 | 0 | Up-reg | 0 | 0 | 0 |
| CCL23 | 0 | 0 | Up-reg | 0 | Up-reg |
| DLG4 | 0 | 0 | Up-reg | Up-reg | Up-reg |
| SPTSSB | 0 | 0 | 0 | 0 | 0 |
| ANXA5 | Up-reg | 0 | Up-reg | Up-reg | Up-reg |
| VAPA | Up-reg | Up-reg | Up-reg | 0 | Up-reg |
| SOGA1 | 0 | 0 | 0 | 0 | 0 |
| CST3 | Up-reg | Up-reg | Up-reg | 0 | Up-reg |
| MAP1LC3A | 0 | Up-reg | 0 | Up-reg | Up-reg |
| MAP9 | 0 | 0 | Up-reg | Up-reg | 0 |
| LGALS1 | 0 | 0 | Up-reg | Up-reg | 0 |
| CCDC149 | 0 | 0 | Up-reg | 0 | 0 |
| GNAS | Up-reg | Up-reg | Up-reg | Up-reg | Up-reg |
| CMBL | 0 | 0 | 0 | 0 | 0 |
| PTPRN | Up-reg | 0 | Up-reg | Up-reg | Up-reg |
| WTIP | 0 | 0 | Up-reg | Up-reg | 0 |
| SPP1 | Up-reg | Up-reg | 0 | Up-reg | Up-reg |
| FXR1 | Up-reg | Up-reg | Up-reg | Up-reg | 0 |
| ARHGEF26 | 0 | 0 | 0 | 0 | 0 |
| PROS1 | 0 | Up-reg | Up-reg | 0 | 0 |
| PARP8 | 0 | Up-reg | Up-reg | 0 | 0 |
| EIF4A2 | Up-reg | Up-reg | 0 | Up-reg | 0 |
| OSR1 | 0 | Up-reg | Up-reg | Up-reg | Up-reg |
| TFF2 | 0 | 0 | Up-reg | 0 | Up-reg |
| ATF4 | Up-reg | 0 | 0 | Up-reg | 0 |
| CTSZ | Up-reg | Up-reg | Up-reg | Up-reg | Up-reg |
| UCHL1 | 0 | 0 | Up-reg | Up-reg | Up-reg |
| ONECUT2 | 0 | 0 | Up-reg | Up-reg | 0 |
| EIF1 | Up-reg | Up-reg | 0 | 0 | Up-reg |
| LAMP2 | Up-reg | Up-reg | Up-reg | Up-reg | Up-reg |
| CALD1 | 0 | Up-reg | Up-reg | Up-reg | Up-reg |
| ATP6V1G1 | Up-reg | Up-reg | 0 | 0 | Up-reg |
| PRSS35 | Up-reg | Up-reg | Up-reg | Up-reg | 0 |
| KCNK5 | 0 | 0 | 0 | Up-reg | 0 |
| CDKN2B | 0 | 0 | Up-reg | Up-reg | Up-reg |
| AEBP1 | 0 | Up-reg | Up-reg | Up-reg | 0 |
| SP8 | Up-reg | Up-reg | Up-reg | 0 | 0 |
| CFTR | Up-reg | 0 | 0 | 0 | 0 |
| TSPAN7 | Up-reg | 0 | Up-reg | 0 | Up-reg |
| MPP6 | Up-reg | Up-reg | 0 | 0 | 0 |
| CYSLTR1 | 0 | 0 | Up-reg | 0 | Up-reg |
| FSCN1 | Up-reg | 0 | Up-reg | Up-reg | Up-reg |
| IL33 | 0 | Up-reg | Up-reg | 0 | Up-reg |
| PLP2 | Up-reg | Up-reg | 0 | 0 | 0 |
| ELFN1 | 0 | Up-reg | Up-reg | Up-reg | Up-reg |
| IGFBP3 | Up-reg | Up-reg | Up-reg | Up-reg | Up-reg |
| SAT1 | 0 | Up-reg | 0 | 0 | Up-reg |
| AFAP1L1 | 0 | 0 | Up-reg | Up-reg | Up-reg |
| LPAR4 | 0 | 0 | Up-reg | Up-reg | 0 |
| ATP6V1F | Up-reg | 0 | 0 | 0 | Up-reg |
| GRINA | Up-reg | 0 | Up-reg | 0 | Up-reg |
| CASD1 | 0 | 0 | Up-reg | Up-reg | 0 |
| HS6ST2 | 0 | 0 | Up-reg | Up-reg | 0 |
| CD109 | Up-reg | 0 | Up-reg | Up-reg | Up-reg |
| PGRMC1 | Up-reg | Up-reg | 0 | 0 | Up-reg |
| MAL2 | 0 | Up-reg | Up-reg | 0 | Up-reg |
| PHF19 | 0 | Up-reg | 0 | 0 | Up-reg |
| TIMP1 | 0 | Up-reg | Up-reg | Up-reg | Up-reg |
| ASAP1 | 0 | Up-reg | Up-reg | Up-reg | Up-reg |

“Up-reg” indicates that a gene is positively regulated by FMRP-activity and “Down-reg” conversely indicates that a gene is negatively regulated by FMRP-activity, and 0 shows the genes not correlated with or regulated by FMRP-activity for this particular cancer type.

| | | Table 21 - | Table 22 - | | |
|---|---|---|---|---|---|
| | Table 20 - | Kidney renal | Kidney renal | Table 23 - | Table 24 - |
| Official | Kidney | clear cell | papillary cell | Bladder | Thyroid |
| Symbol | Chromophobe | carcinoma | carcinoma | Carcinoma | carcinoma |
| EIF4G3 | 0 | 0 | 0 | 0 | 0 |
| SMPDL3B | Down-reg | 0 | 0 | Down-reg | Down-reg |
| VANGL2 | Down-reg | 0 | Down-reg | Down-reg | Down-reg |
| GBP2 | 0 | 0 | 0 | Down-reg | Down-reg |
| POGK | 0 | 0 | 0 | Down-reg | 0 |
| IFITM2 | 0 | 0 | 0 | 0 | Down-reg |
| IFITM1 | Down-reg | 0 | 0 | 0 | Down-reg |
| IFITM3 | Down-reg | 0 | 0 | 0 | Down-reg |
| PDLIM1 | Down-reg | 0 | Down-reg | Down-reg | Down-reg |
| PRDX5 | Down-reg | 0 | Down-reg | Down-reg | Down-reg |
| PFKP | 0 | Down-reg | Down-reg | 0 | 0 |
| SIPA1L2 | 0 | Down-reg | 0 | 0 | Down-reg |
| ACSL5 | Down-reg | 0 | Down-reg | Down-reg | Down-reg |
| RBP4 | 0 | Down-reg | Down-reg | 0 | 0 |
| BNC1 | Down-reg | 0 | Down-reg | 0 | Down-reg |
| PSME2 | 0 | 0 | 0 | Down-reg | Down-reg |
| B2M | 0 | Down-reg | 0 | Down-reg | Down-reg |
| GAS6 | Down-reg | 0 | Down-reg | 0 | Down-reg |
| PSME1 | Down-reg | 0 | 0 | Down-reg | Down-reg |
| CKMT1B | Down-reg | 0 | Down-reg | Down-reg | 0 |
| CKMT1A | Down-reg | 0 | Down-reg | 0 | 0 |
| WDR89 | 0 | Down-reg | 0 | 0 | Down-reg |
| USP50 | 0 | 0 | 0 | 0 | 0 |
| CRIP1 | 0 | 0 | Down-reg | 0 | 0 |
| CHCHD10 | Down-reg | Down-reg | Down-reg | Down-reg | 0 |
| ZNF23 | Down-reg | 0 | Down-reg | Down-reg | 0 |
| APOB | 0 | 0 | Down-reg | 0 | 0 |
| UBA52 | 0 | 0 | 0 | Down-reg | Down-reg |
| POGLUT1 | 0 | 0 | 0 | 0 | 0 |
| PLAC8 | 0 | 0 | 0 | Down-reg | 0 |
| STAT1 | 0 | 0 | 0 | Down-reg | Down-reg |
| PDE5A | 0 | Down-reg | 0 | 0 | Down-reg |
| CPEB2 | Down-reg | Down-reg | Down-reg | 0 | Down-reg |
| PCDHB11 | Down-reg | 0 | 0 | 0 | 0 |
| PCDHB12 | 0 | 0 | Down-reg | 0 | Down-reg |
| PCDHB15 | 0 | Down-reg | Down-reg | 0 | Down-reg |
| ATP13A4 | Down-reg | Down-reg | Down-reg | 0 | Down-reg |
| HMGB2 | 0 | 0 | 0 | Down-reg | Down-reg |
| RPL29 | 0 | 0 | 0 | Down-reg | Down-reg |
| PPARGC1A | Down-reg | Down-reg | Down-reg | 0 | 0 |
| CHN1 | Down-reg | 0 | 0 | 0 | Down-reg |
| CCL8 | 0 | 0 | 0 | 0 | Down-reg |
| SLC4A4 | 0 | Down-reg | 0 | Down-reg | 0 |
| LSM4 | 0 | 0 | Down-reg | Down-reg | Down-reg |
| KIAA0513 | Down-reg | Down-reg | Down-reg | 0 | 0 |
| NME1 | 0 | 0 | 0 | 0 | Down-reg |
| BST2 | 0 | 0 | 0 | Down-reg | Down-reg |
| TMEM144 | 0 | Down-reg | 0 | Down-reg | 0 |
| COL3A1 | 0 | 0 | 0 | 0 | 0 |
| PSMB10 | Down-reg | 0 | Down-reg | Down-reg | Down-reg |
| MB21D2 | 0 | 0 | 0 | 0 | 0 |
| ZDHHC23 | 0 | 0 | Down-reg | Down-reg | 0 |
| MT2A | 0 | 0 | 0 | 0 | 0 |
| TFAP2A | Down-reg | 0 | 0 | 0 | 0 |
| PARP12 | 0 | 0 | 0 | Down-reg | Down-reg |
| HSPB1 | Down-reg | 0 | 0 | 0 | 0 |
| HNRNPA2B1 | 0 | 0 | 0 | Down-reg | Down-reg |
| ENTPD2 | Down-reg | Down-reg | Down-reg | 0 | Down-reg |
| MYLIP | Down-reg | Down-reg | Down-reg | Down-reg | Down-reg |
| MTMR7 | 0 | 0 | Down-reg | Down-reg | Down-reg |
| PSMB8 | 0 | 0 | Down-reg | Down-reg | Down-reg |
| AUTS2 | Down-reg | Down-reg | Down-reg | Down-reg | Down-reg |
| UPP1 | 0 | 0 | 0 | 0 | Down-reg |
| TAPBP | Down-reg | 0 | Down-reg | Down-reg | 0 |
| KLRG2 | 0 | 0 | Down-reg | 0 | 0 |
| PSMB9 | Down-reg | 0 | 0 | Down-reg | Down-reg |
| MARCKSL1 | Up-reg | Up-reg | Up-reg | 0 | Up-reg |
| ID3 | 0 | Up-reg | 0 | 0 | Up-reg |
| S100A16 | Up-reg | Up-reg | Up-reg | Up-reg | 0 |
| PLPP3 | 0 | 0 | 0 | 0 | 0 |
| GADD45A | Up-reg | 0 | 0 | 0 | 0 |
| S100A4 | Up-reg | Up-reg | 0 | 0 | Up-reg |
| DDAH1 | Up-reg | 0 | 0 | 0 | 0 |
| MYCL | 0 | 0 | 0 | 0 | 0 |
| CD81 | 0 | Up-reg | Up-reg | 0 | 0 |
| SHANK2 | 0 | 0 | 0 | 0 | Up-reg |
| ITIH2 | 0 | Up-reg | Up-reg | 0 | Up-reg |
| PIK3AP1 | Up-reg | Up-reg | 0 | 0 | 0 |
| LHFPL6 | 0 | 0 | 0 | 0 | 0 |
| LGALS3 | 0 | Up-reg | 0 | Up-reg | 0 |
| FRMD5 | Up-reg | Up-reg | 0 | 0 | 0 |
| CLDN6 | Up-reg | Up-reg | Up-reg | Up-reg | 0 |
| TNFRSF12A | 0 | Up-reg | 0 | Up-reg | 0 |
| NPC2 | 0 | Up-reg | 0 | 0 | 0 |
| CD9 | 0 | 0 | 0 | 0 | 0 |
| ATP11A | Up-reg | 0 | 0 | Up-reg | 0 |
| SLC25A21 | Up-reg | 0 | 0 | Up-reg | Up-reg |
| CD63 | Up-reg | Up-reg | Up-reg | 0 | 0 |
| B4GALNT3 | 0 | Up-reg | Up-reg | 0 | 0 |
| EMP1 | Up-reg | 0 | 0 | Up-reg | 0 |
| CSTB | 0 | Up-reg | Up-reg | 0 | 0 |
| WNT10A | 0 | Up-reg | Up-reg | 0 | 0 |
| H3-3B | 0 | 0 | 0 | 0 | 0 |
| RABAC1 | 0 | Up-reg | Up-reg | 0 | Up-reg |
| KCTD17 | Up-reg | Up-reg | Up-reg | 0 | 0 |
| BCAM | 0 | 0 | 0 | 0 | 0 |
| CCL15-CCL14 | 0 | 0 | 0 | 0 | 0 |
| CCL15 | Up-reg | 0 | 0 | 0 | Up-reg |
| CCL23 | Up-reg | Up-reg | Up-reg | 0 | 0 |
| DLG4 | Up-reg | Up-reg | 0 | Up-reg | 0 |
| SPTSSB | 0 | 0 | 0 | 0 | 0 |
| ANXA5 | Up-reg | Up-reg | Up-reg | Up-reg | 0 |
| VAPA | 0 | 0 | 0 | Up-reg | 0 |
| SOGA1 | 0 | 0 | 0 | 0 | 0 |
| CST3 | 0 | Up-reg | 0 | 0 | 0 |
| MAP1LC3A | 0 | Up-reg | 0 | 0 | Up-reg |
| MAP9 | 0 | Up-reg | 0 | Up-reg | Up-reg |
| LGALS1 | Up-reg | Up-reg | Up-reg | Up-reg | 0 |
| CCDC149 | Up-reg | 0 | 0 | 0 | Up-reg |
| GNAS | 0 | Up-reg | 0 | Up-reg | Up-reg |
| CMBL | 0 | 0 | Up-reg | 0 | Up-reg |
| PTPRN | Up-reg | Up-reg | Up-reg | 0 | 0 |
| WTIP | 0 | 0 | 0 | 0 | 0 |
| SPP1 | Up-reg | Up-reg | Up-reg | Up-reg | Up-reg |
| FXR1 | Up-reg | 0 | Up-reg | Up-reg | Up-reg |
| ARHGEF26 | 0 | 0 | 0 | 0 | 0 |
| PROS1 | 0 | 0 | Up-reg | Up-reg | Up-reg |
| PARP8 | 0 | 0 | 0 | 0 | 0 |
| EIF4A2 | 0 | Up-reg | Up-reg | 0 | Up-reg |
| OSR1 | Up-reg | Up-reg | 0 | 0 | Up-reg |
| TFF2 | 0 | 0 | 0 | 0 | 0 |
| ATF4 | Up-reg | Up-reg | Up-reg | 0 | 0 |
| CTSZ | Up-reg | Up-reg | 0 | 0 | 0 |
| UCHL1 | Up-reg | Up-reg | Up-reg | Up-reg | Up-reg |
| ONECUT2 | Up-reg | Up-reg | 0 | Up-reg | 0 |
| EIF1 | Up-reg | 0 | Up-reg | Up-reg | 0 |
| LAMP2 | Up-reg | 0 | Up-reg | Up-reg | 0 |
| CALD1 | Up-reg | 0 | Up-reg | Up-reg | Up-reg |
| ATP6V1G1 | Up-reg | 0 | 0 | 0 | 0 |
| PRSS35 | Up-reg | Up-reg | Up-reg | Up-reg | Up-reg |
| KCNK5 | 0 | 0 | 0 | 0 | 0 |
| CDKN2B | 0 | 0 | Up-reg | 0 | 0 |
| AEBP1 | Up-reg | Up-reg | Up-reg | Up-reg | Up-reg |
| SP8 | 0 | 0 | 0 | 0 | 0 |
| CFTR | Up-reg | 0 | 0 | 0 | 0 |
| TSPAN7 | 0 | 0 | Up-reg | Up-reg | Up-reg |
| MPP6 | Up-reg | 0 | Up-reg | Up-reg | Up-reg |
| CYSLTR1 | 0 | 0 | Up-reg | 0 | Up-reg |
| FSCN1 | Up-reg | Up-reg | 0 | Up-reg | Up-reg |
| IL33 | 0 | 0 | Up-reg | Up-reg | Up-reg |
| PLP2 | Up-reg | Up-reg | Up-reg | 0 | Up-reg |
| ELFN1 | 0 | 0 | Up-reg | 0 | 0 |
| IGFBP3 | Up-reg | Up-reg | Up-reg | 0 | Up-reg |
| SAT1 | 0 | Up-reg | 0 | 0 | 0 |
| AFAP1L1 | Up-reg | 0 | Up-reg | Up-reg | 0 |
| LPAR4 | 0 | 0 | Up-reg | Up-reg | Up-reg |
| ATP6V1F | Up-reg | Up-reg | 0 | 0 | 0 |
| GRINA | 0 | Up-reg | Up-reg | 0 | Up-reg |
| CASD1 | Up-reg | 0 | Up-reg | 0 | 0 |
| HS6ST2 | 0 | Up-reg | Up-reg | 0 | 0 |
| CD109 | Up-reg | 0 | 0 | Up-reg | 0 |
| PGRMC1 | Up-reg | 0 | Up-reg | 0 | 0 |
| MAL2 | 0 | 0 | 0 | 0 | Up-reg |
| PHF19 | Up-reg | Up-reg | 0 | Up-reg | 0 |
| TIMP1 | Up-reg | Up-reg | Up-reg | 0 | 0 |
| ASAP1 | Up-reg | 0 | Up-reg | Up-reg | Up-reg |

“Up-reg” indicates that a gene is positively regulated by FMRP-activity and “Down-reg” conversely indicates that a gene is negatively regulated by FMRP-activity, and 0 shows the genes not correlated with or regulated by FMRP-activity for this particular cancer type.

| Official | Table 25 - | Table 26 - | Table 27 - | Table 28 - |
|---|---|---|---|---|
| Symbol | Glioblastoma | Glioma | Sarcoma | Melanoma |
| EIF4G3 | Down-reg | 0 | 0 | 0 |
| SMPDL3B | 0 | 0 | Down-reg | Down-reg |
| VANGL2 | Down-reg | Down-reg | Down-reg | 0 |
| GBP2 | Down-reg | 0 | Down-reg | Down-reg |
| POGK | Down-reg | Down-reg | 0 | 0 |
| IFITM2 | 0 | 0 | Down-reg | Down-reg |
| IFITM1 | 0 | 0 | Down-reg | Down-reg |
| IFITM3 | 0 | 0 | Down-reg | Down-reg |
| PDLIM1 | 0 | 0 | Down-reg | 0 |
| PRDX5 | Down-reg | Down-reg | Down-reg | 0 |
| PFKP | 0 | Down-reg | Down-reg | 0 |
| SIPA1L2 | 0 | 0 | Down-reg | Down-reg |
| ACSL5 | 0 | 0 | Down-reg | Down-reg |
| RBP4 | 0 | Down-reg | 0 | 0 |
| BNC1 | 0 | Down-reg | 0 | 0 |
| PSME2 | 0 | 0 | Down-reg | Down-reg |
| B2M | 0 | 0 | Down-reg | Down-reg |
| GAS6 | 0 | 0 | 0 | 0 |
| PSME1 | 0 | Down-reg | Down-reg | Down-reg |
| CKMT1B | 0 | Down-reg | 0 | 0 |
| CKMT1A | Down-reg | Down-reg | 0 | 0 |
| WDR89 | Down-reg | Down-reg | 0 | Down-reg |
| USP50 | 0 | 0 | 0 | 0 |
| CRIP1 | 0 | 0 | Down-reg | Down-reg |
| CHCHD10 | 0 | 0 | Down-reg | 0 |
| ZNF23 | Down-reg | 0 | 0 | 0 |
| APOB | 0 | 0 | Down-reg | 0 |
| UBA52 | Down-reg | 0 | 0 | 0 |
| POGLUT1 | 0 | 0 | 0 | 0 |
| PLAC8 | 0 | 0 | Down-reg | Down-reg |
| STAT1 | Down-reg | 0 | Down-reg | Down-reg |
| PDE5A | Down-reg | 0 | Down-reg | Down-reg |
| CPEB2 | 0 | 0 | 0 | 0 |
| PCDHB11 | 0 | 0 | 0 | 0 |
| PCDHB12 | 0 | 0 | 0 | 0 |
| PCDHB15 | 0 | 0 | Down-reg | Down-reg |
| ATP13A4 | Down-reg | Down-reg | 0 | Down-reg |
| HMGB2 | Down-reg | 0 | 0 | Down-reg |
| RPL29 | Down-reg | Down-reg | 0 | 0 |
| PPARGC1A | 0 | 0 | Down-reg | 0 |
| CHN1 | 0 | 0 | 0 | Down-reg |
| CCL8 | 0 | 0 | Down-reg | Down-reg |
| SLC4A4 | 0 | Down-reg | 0 | Down-reg |
| LSM4 | 0 | 0 | 0 | 0 |
| KIAA0513 | 0 | Down-reg | 0 | Down-reg |
| NME1 | 0 | 0 | 0 | 0 |
| BST2 | 0 | 0 | Down-reg | Down-reg |
| TMEM144 | 0 | 0 | Down-reg | 0 |
| COL3A1 | 0 | 0 | 0 | 0 |
| PSMB10 | 0 | 0 | Down-reg | Down-reg |
| MB21D2 | 0 | 0 | 0 | 0 |
| ZDHHC23 | 0 | 0 | 0 | Down-reg |
| MT2A | 0 | 0 | 0 | Down-reg |
| TFAP2A | 0 | 0 | 0 | 0 |
| PARP12 | 0 | 0 | Down-reg | Down-reg |
| HSPB1 | 0 | 0 | Down-reg | 0 |
| HNRNPA2B1 | Down-reg | 0 | 0 | 0 |
| ENTPD2 | 0 | 0 | Down-reg | 0 |
| MYLIP | Down-reg | Down-reg | Down-reg | 0 |
| MTMR7 | Down-reg | Down-reg | 0 | Down-reg |
| PSMB8 | 0 | 0 | Down-reg | Down-reg |
| AUTS2 | 0 | 0 | 0 | Down-reg |
| UPP1 | 0 | 0 | 0 | 0 |
| TAPBP | 0 | 0 | Down-reg | Down-reg |
| KLRG2 | 0 | 0 | 0 | 0 |
| PSMB9 | Down-reg | 0 | Down-reg | Down-reg |
| MARCKSL1 | 0 | 0 | Up-reg | Up-reg |
| ID3 | 0 | Up-reg | Up-reg | 0 |
| S100A16 | Up-reg | Up-reg | 0 | 0 |
| PLPP3 | 0 | 0 | 0 | 0 |
| GADD45A | 0 | Up-reg | Up-reg | 0 |
| S100A4 | Up-reg | Up-reg | 0 | 0 |
| DDAH1 | 0 | Up-reg | Up-reg | 0 |
| MYCL | 0 | 0 | 0 | 0 |
| CD81 | Up-reg | 0 | 0 | Up-reg |
| SHANK2 | 0 | 0 | Up-reg | Up-reg |
| ITIH2 | 0 | 0 | 0 | 0 |
| PIK3AP1 | Up-reg | Up-reg | 0 | 0 |
| LHFPL6 | 0 | 0 | 0 | 0 |
| LGALS3 | Up-reg | Up-reg | 0 | 0 |
| FRMD5 | 0 | 0 | Up-reg | 0 |
| CLDN6 | Up-reg | Up-reg | 0 | 0 |
| TNFRSF12A | Up-reg | Up-reg | Up-reg | 0 |
| NPC2 | Up-reg | Up-reg | 0 | 0 |
| CD9 | 0 | Up-reg | Up-reg | 0 |
| ATP11A | 0 | Up-reg | Up-reg | Up-reg |
| SLC25A21 | 0 | 0 | 0 | 0 |
| CD63 | Up-reg | Up-reg | 0 | Up-reg |
| B4GALNT3 | 0 | Up-reg | 0 | Up-reg |
| EMP1 | Up-reg | Up-reg | Up-reg | Up-reg |
| CSTB | Up-reg | Up-reg | Up-reg | Up-reg |
| WNT10A | Up-reg | Up-reg | 0 | 0 |
| H3-3B | 0 | 0 | 0 | 0 |
| RABAC1 | 0 | Up-reg | 0 | 0 |
| KCTD17 | Up-reg | Up-reg | 0 | 0 |
| BCAM | Up-reg | Up-reg | 0 | 0 |
| CCL15-CCL14 | 0 | 0 | 0 | 0 |
| CCL15 | 0 | 0 | 0 | 0 |
| CCL23 | Up-reg | 0 | 0 | 0 |
| DLG4 | Up-reg | 0 | Up-reg | 0 |
| SPTSSB | 0 | 0 | 0 | 0 |
| ANXA5 | Up-reg | Up-reg | Up-reg | Up-reg |
| VAPA | 0 | 0 | 0 | 0 |
| SOGA1 | 0 | 0 | 0 | 0 |
| CST3 | Up-reg | Up-reg | 0 | 0 |
| MAP1LC3A | Up-reg | Up-reg | 0 | 0 |
| MAP9 | Up-reg | 0 | 0 | 0 |
| LGALS1 | Up-reg | Up-reg | Up-reg | Up-reg |
| CCDC149 | Up-reg | 0 | 0 | Up-reg |
| GNAS | 0 | 0 | Up-reg | 0 |
| CMBL | Up-reg | 0 | 0 | 0 |
| PTPRN | Up-reg | 0 | Up-reg | 0 |
| WTIP | Up-reg | Up-reg | Up-reg | Up-reg |
| SPP1 | Up-reg | Up-reg | Up-reg | 0 |
| FXR1 | 0 | 0 | Up-reg | 0 |
| ARHGEF26 | 0 | 0 | 0 | 0 |
| PROS1 | Up-reg | Up-reg | 0 | Up-reg |
| PARP8 | 0 | 0 | 0 | 0 |
| EIF4A2 | 0 | 0 | 0 | 0 |
| OSR1 | 0 | Up-reg | 0 | 0 |
| TFF2 | 0 | 0 | 0 | 0 |
| ATF4 | 0 | 0 | 0 | 0 |
| CTSZ | Up-reg | Up-reg | 0 | 0 |
| UCHL1 | Up-reg | Up-reg | 0 | 0 |
| ONECUT2 | 0 | 0 | Up-reg | 0 |
| EIF1 | Up-reg | 0 | 0 | 0 |
| LAMP2 | Up-reg | Up-reg | Up-reg | 0 |
| CALD1 | Up-reg | Up-reg | 0 | 0 |
| ATP6V1G1 | 0 | 0 | Up-reg | 0 |
| PRSS35 | 0 | 0 | Up-reg | 0 |
| KCNK5 | Up-reg | Up-reg | 0 | 0 |
| CDKN2B | 0 | 0 | 0 | Up-reg |
| AEBP1 | Up-reg | Up-reg | 0 | Up-reg |
| SP8 | 0 | Up-reg | 0 | Up-reg |
| CFTR | 0 | 0 | 0 | 0 |
| TSPAN7 | 0 | 0 | 0 | 0 |
| MPP6 | Up-reg | 0 | Up-reg | Up-reg |
| CYSLTR1 | 0 | Up-reg | 0 | 0 |
| FSCN1 | Up-reg | Up-reg | Up-reg | 0 |
| IL33 | Up-reg | 0 | 0 | 0 |
| PLP2 | Up-reg | Up-reg | 0 | 0 |
| ELFN1 | Up-reg | 0 | 0 | Up-reg |
| IGFBP3 | Up-reg | Up-reg | Up-reg | 0 |
| SAT1 | Up-reg | Up-reg | 0 | 0 |
| AFAP1L1 | 0 | Up-reg | 0 | 0 |
| LPAR4 | 0 | 0 | Up-reg | 0 |
| ATP6V1F | Up-reg | Up-reg | 0 | Up-reg |
| GRINA | Up-reg | 0 | 0 | Up-reg |
| CASD1 | Up-reg | 0 | Up-reg | 0 |
| HS6ST2 | 0 | 0 | Up-reg | 0 |
| CD109 | Up-reg | Up-reg | Up-reg | 0 |
| PGRMC1 | Up-reg | 0 | Up-reg | 0 |
| MAL2 | 0 | 0 | Up-reg | Up-reg |
| PHF19 | 0 | Up-reg | 0 | Up-reg |
| TIMP1 | Up-reg | Up-reg | 0 | 0 |
| ASAP1 | 0 | Up-reg | Up-reg | 0 |

“Up-reg” indicates that a gene is positively regulated by FMRP-activity and Down-reg” conversely indicates that a gene is negatively regulated by FMRP-activity.”, and 0 shows the genes not correlated with or regulated by FMRP-activity for this particular cancer type.

| | | | | Table 32 - |
|---|---|---|---|---|
| | | | Table 31 - | Pheochro- |
| | | | Adreno- | mocytoma |
| Official | Table 29 - | Table 30 - | cortical | and Para- |
| Symbol | Leukemia | Thymoma | carcinoma | ganglioma |
| EIF4G3 | 0 | Down-reg | 0 | Down-reg |
| SMPDL3B | 0 | 0 | 0 | 0 |
| VANGL2 | Down-reg | 0 | 0 | 0 |
| GBP2 | 0 | 0 | Down-reg | Down-reg |
| POGK | 0 | Down-reg | 0 | 0 |
| IFITM2 | 0 | 0 | Down-reg | Down-reg |
| IFITM1 | 0 | 0 | Down-reg | Down-reg |
| IFITM3 | 0 | 0 | Down-reg | Down-reg |
| PDLIM1 | 0 | 0 | 0 | 0 |
| PRDX5 | 0 | Down-reg | Down-reg | 0 |
| PFKP | 0 | 0 | 0 | 0 |
| SIPA1L2 | 0 | Down-reg | 0 | Down-reg |
| ACSL5 | 0 | 0 | Down-reg | Down-reg |
| RBP4 | 0 | 0 | Down-reg | 0 |
| BNC1 | 0 | 0 | Down-reg | Down-reg |
| PSME2 | 0 | 0 | 0 | 0 |
| B2M | Down-reg | 0 | Down-reg | Down-reg |
| GAS6 | 0 | Down-reg | Down-reg | Down-reg |
| PSME1 | 0 | 0 | Down-reg | 0 |
| CKMT1B | 0 | 0 | Down-reg | 0 |
| CKMT1A | 0 | 0 | Down-reg | Down-reg |
| WDR89 | 0 | Down-reg | Down-reg | 0 |
| USP50 | 0 | 0 | 0 | 0 |
| CRIP1 | 0 | Down-reg | 0 | Down-reg |
| CHCHD10 | 0 | 0 | 0 | Down-reg |
| ZNF23 | Down-reg | Down-reg | Down-reg | 0 |
| APOB | 0 | Down-reg | 0 | Down-reg |
| UBA52 | Down-reg | Down-reg | 0 | Down-reg |
| POGLUT1 | 0 | 0 | 0 | 0 |
| PLAC8 | Down-reg | 0 | Down-reg | Down-reg |
| STAT1 | 0 | 0 | 0 | Down-reg |
| PDE5A | Down-reg | Down-reg | 0 | Down-reg |
| CPEB2 | 0 | 0 | Down-reg | Down-reg |
| PCDHB11 | Down-reg | 0 | Down-reg | Down-reg |
| PCDHB12 | Down-reg | 0 | Down-reg | 0 |
| PCDHB15 | 0 | 0 | 0 | Down-reg |
| ATP13A4 | Down-reg | Down-reg | Down-reg | Down-reg |
| HMGB2 | Down-reg | Down-reg | 0 | 0 |
| RPL29 | 0 | 0 | 0 | Down-reg |
| PPARGC1A | 0 | Down-reg | Down-reg | 0 |
| CHN1 | 0 | 0 | 0 | Down-reg |
| CCL8 | 0 | 0 | Down-reg | Down-reg |
| SLC4A4 | 0 | 0 | Down-reg | Down-reg |
| LSM4 | 0 | Down-reg | 0 | Down-reg |
| KIAA0513 | 0 | 0 | Down-reg | Down-reg |
| NME1 | 0 | 0 | 0 | Down-reg |
| BST2 | 0 | Down-reg | Down-reg | Down-reg |
| TMEM144 | 0 | 0 | Down-reg | Down-reg |
| COL3A1 | Down-reg | Down-reg | 0 | 0 |
| PSMB10 | 0 | 0 | Down-reg | Down-reg |
| MB21D2 | 0 | 0 | 0 | 0 |
| ZDHHC23 | Down-reg | 0 | 0 | 0 |
| MT2A | 0 | 0 | Down-reg | 0 |
| TFAP2A | Down-reg | Down-reg | 0 | 0 |
| PARP12 | 0 | 0 | Down-reg | Down-reg |
| HSPB1 | 0 | Down-reg | Down-reg | Down-reg |
| HNRNPA2B1 | 0 | Down-reg | 0 | 0 |
| ENTPD2 | 0 | Down-reg | 0 | 0 |
| MYLIP | Down-reg | Down-reg | Down-reg | Down-reg |
| MTMR7 | Down-reg | Down-reg | Down-reg | Down-reg |
| PSMB8 | 0 | 0 | Down-reg | 0 |
| AUTS2 | Down-reg | Down-reg | 0 | 0 |
| UPP1 | 0 | 0 | 0 | 0 |
| TAPBP | 0 | 0 | Down-reg | Down-reg |
| KLRG2 | Down-reg | Down-reg | 0 | 0 |
| PSMB9 | 0 | 0 | Down-reg | 0 |
| MARCKSL1 | 0 | Up-reg | Up-reg | 0 |
| ID3 | 0 | 0 | 0 | 0 |
| S100A16 | 0 | Up-reg | 0 | Up-reg |
| PLPP3 | 0 | 0 | 0 | 0 |
| GADD45A | Up-reg | Up-reg | Up-reg | Up-reg |
| S100A4 | Up-reg | Up-reg | 0 | Up-reg |
| DDAH1 | Up-reg | Up-reg | Up-reg | 0 |
| MYCL | 0 | 0 | 0 | 0 |
| CD81 | 0 | 0 | Up-reg | Up-reg |
| SHANK2 | 0 | Up-reg | Up-reg | 0 |
| ITIH2 | 0 | Up-reg | Up-reg | Up-reg |
| PIK3AP1 | Up-reg | Up-reg | 0 | 0 |
| LHFPL6 | 0 | 0 | 0 | 0 |
| LGALS3 | Up-reg | Up-reg | Up-reg | Up-reg |
| FRMD5 | 0 | 0 | Up-reg | Up-reg |
| CLDN6 | 0 | 0 | Up-reg | 0 |
| TNFRSF12A | 0 | 0 | 0 | 0 |
| NPC2 | 0 | Up-reg | 0 | Up-reg |
| CD9 | 0 | Up-reg | 0 | 0 |
| ATP11A | 0 | Up-reg | Up-reg | Up-reg |
| SLC25A21 | 0 | Up-reg | 0 | 0 |
| CD63 | 0 | Up-reg | 0 | 0 |
| B4GALNT3 | 0 | Up-reg | Up-reg | Up-reg |
| EMP1 | Up-reg | Up-reg | 0 | 0 |
| CSTB | Up-reg | Up-reg | 0 | Up-reg |
| WNT10A | 0 | Up-reg | Up-reg | 0 |
| H3-3B | 0 | 0 | 0 | 0 |
| RABAC1 | Up-reg | 0 | Up-reg | Up-reg |
| KCTD17 | Up-reg | Up-reg | Up-reg | 0 |
| BCAM | 0 | 0 | Up-reg | 0 |
| CCL15-CCL14 | 0 | 0 | 0 | 0 |
| CCL15 | 0 | 0 | 0 | 0 |
| CCL23 | Up-reg | Up-reg | 0 | 0 |
| DLG4 | 0 | 0 | Up-reg | Up-reg |
| SPTSSB | 0 | 0 | 0 | 0 |
| ANXA5 | Up-reg | Up-reg | 0 | 0 |
| VAPA | 0 | 0 | Up-reg | 0 |
| SOGA1 | 0 | 0 | 0 | 0 |
| CST3 | 0 | Up-reg | 0 | Up-reg |
| MAP1LC3A | 0 | Up-reg | Up-reg | 0 |
| MAP9 | Up-reg | Up-reg | 0 | 0 |
| LGALS1 | Up-reg | 0 | Up-reg | 0 |
| CCDC149 | 0 | Up-reg | 0 | 0 |
| GNAS | 0 | 0 | 0 | 0 |
| CMBL | 0 | Up-reg | 0 | Up-reg |
| PTPRN | 0 | Up-reg | 0 | Up-reg |
| WTIP | 0 | 0 | Up-reg | 0 |
| SPP1 | 0 | Up-reg | Up-reg | Up-reg |
| FXR1 | 0 | 0 | Up-reg | Up-reg |
| ARHGEF26 | 0 | 0 | 0 | 0 |
| PROS1 | Up-reg | 0 | 0 | 0 |
| PARP8 | 0 | Up-reg | 0 | 0 |
| EIF4A2 | 0 | Up-reg | 0 | 0 |
| OSR1 | 0 | 0 | Up-reg | Up-reg |
| TFF2 | 0 | 0 | 0 | 0 |
| ATF4 | 0 | Up-reg | Up-reg | 0 |
| CTSZ | 0 | Up-reg | Up-reg | 0 |
| UCHL1 | Up-reg | Up-reg | 0 | Up-reg |
| ONECUT2 | Up-reg | Up-reg | Up-reg | Up-reg |
| EIF1 | 0 | 0 | Up-reg | 0 |
| LAMP2 | 0 | Up-reg | 0 | 0 |
| CALD1 | 0 | 0 | 0 | 0 |
| ATP6V1G1 | 0 | 0 | Up-reg | 0 |
| PRSS35 | 0 | 0 | Up-reg | Up-reg |
| KCNK5 | Up-reg | 0 | Up-reg | Up-reg |
| CDKN2B | 0 | Up-reg | Up-reg | Up-reg |
| AEBP1 | 0 | 0 | Up-reg | 0 |
| SP8 | 0 | 0 | 0 | 0 |
| CFTR | 0 | 0 | 0 | 0 |
| TSPAN7 | 0 | 0 | Up-reg | Up-reg |
| MPP6 | 0 | Up-reg | 0 | Up-reg |
| CYSLTR1 | 0 | Up-reg | 0 | 0 |
| FSCN1 | 0 | Up-reg | Up-reg | 0 |
| IL33 | 0 | Up-reg | 0 | 0 |
| PLP2 | 0 | 0 | 0 | 0 |
| ELFN1 | 0 | 0 | Up-reg | 0 |
| IGFBP3 | 0 | Up-reg | Up-reg | Up-reg |
| SAT1 | Up-reg | Up-reg | 0 | 0 |
| AFAP1L1 | 0 | Up-reg | Up-reg | 0 |
| LPAR4 | 0 | 0 | Up-reg | 0 |
| ATP6V1F | 0 | Up-reg | 0 | Up-reg |
| GRINA | Up-reg | Up-reg | 0 | 0 |
| CASD1 | 0 | Up-reg | 0 | 0 |
| HS6ST2 | 0 | 0 | Up-reg | Up-reg |
| CD109 | Up-reg | 0 | 0 | 0 |
| PGRMC1 | Up-reg | Up-reg | Up-reg | 0 |
| MAL2 | 0 | Up-reg | 0 | 0 |
| PHF19 | 0 | 0 | Up-reg | Up-reg |
| TIMP1 | 0 | Up-reg | 0 | 0 |
| ASAP1 | 0 | Up-reg | Up-reg | Up-reg |

“Up-reg” indicates that a gene is positively regulated by FMRP-activity and “Down-reg” conversely indicates that a gene is negatively regulated by FMRP-activity.”, and 0 shows the genes not correlated with or regulated by FMRP-activity for this particular cancer type.

### Pan-Immunosuppressive Signature

In one embodiment, the invention provides an independent pan-cancer “FMRP immunosuppression” gene signature, referred to herein as the Pan-Immunosuppression signature. The Pan-Immunosuppression signature is based on short-term FMRP knock-out in cultured cells and can be used for developing a gene expression signature score that evaluates the level of immunosuppression induced by FMRP-activity and represents the level of CD8 infiltration in tumors at pan-cancer level, as well as a verity of specific cancer types.

The Pan-Immunosuppression signature is an overarching signature list comprising the full panel of biomarker genes (195 genes in total) discovered by comparing FMRP active vs. FMRP knock-out (by siRNA and hence inactive) cultured cancer cells. Pan-Immunosuppression signature is disclosed in Table 33.

TABLE 33

| Official | | Secreted | Up/Down-regulation |
|---|---|---|---|
| Symbol | ensembl_gene_id | proteins | by FMRP |
| MRC1 | ENSG00000260314 | | Down-reg |
| KDELR3 | ENSG00000100196 | | Down-reg |
| SLC7A1 | ENSG00000139514 | | Down-reg |
| PIK3CD | ENSG00000171608 | | Down-reg |
| BCAT1 | ENSG00000060982 | | Down-reg |
| JDP2 | ENSG00000140044 | | Down-reg |
| ADGRA2 | ENSG00000020181 | | Down-reg |
| HMOX1 | ENSG00000100292 | | Down-reg |
| COBL | ENSG00000106078 | | Down-reg |
| PSAT1 | ENSG00000135069 | | Down-reg |
| CHD5 | ENSG00000116254 | | Down-reg |
| CHAC1 | ENSG00000128965 | | Down-reg |
| ATP2A3 | ENSG00000074370 | | Down-reg |
| EIF4EBP1 | ENSG00000187840 | | Down-reg |
| CA6 | ENSG00000131686 | Secretome | Down-reg |
| AVIL | ENSG00000135407 | | Down-reg |
| PSPH | ENSG00000146733 | | Down-reg |
| HMGA1 | ENSG00000137309 | | Down-reg |
| ATF4 | ENSG00000128272 | | Down-reg |
| SLC1A4 | ENSG00000115902 | | Down-reg |
| CIART | ENSG00000159208 | | Down-reg |
| TRIB3 | ENSG00000101255 | | Down-reg |
| LIMS4 | ENSG00000256671 | | Down-reg |
| AREG | ENSG00000109321 | Secretome | Down-reg |
| IFRD1 | ENSG00000006652 | | Down-reg |
| SLC7A11 | ENSG00000151012 | | Down-reg |
| ASNS | ENSG00000070669 | | Down-reg |
| ACAT2 | ENSG00000120437 | | Down-reg |
| LHFPL2 | ENSG00000145685 | | Down-reg |
| EXTL1 | ENSG00000158008 | | Down-reg |
| FOSL1 | ENSG00000175592 | | Down-reg |
| CDSN | ENSG00000204539 | Secretome | Down-reg |
| SNAI2 | ENSG00000019549 | | Down-reg |
| ALDH1L2 | ENSG00000136010 | | Down-reg |
| SLC7A5 | ENSG00000103257 | | Down-reg |
| TMEM266 | ENSG00000169758 | | Down-reg |
| PCK2 | ENSG00000100889 | | Down-reg |
| PHF19 | ENSG00000119403 | | Down-reg |
| FTL | ENSG00000087086 | | Down-reg |
| GRAMD2A | ENSG00000175318 | | Down-reg |
| CPS1 | ENSG00000021826 | | Down-reg |
| CAV1 | ENSG00000105974 | | Down-reg |
| UNC13C | ENSG00000137766 | | Down-reg |
| BEND6 | ENSG00000151917 | | Down-reg |
| TIGIT | ENSG00000181847 | Secretome | Down-reg |
| YARS1 | ENSG00000134684 | | Down-reg |
| LIMS3 | ENSG00000256977 | | Down-reg |
| STBD1 | ENSG00000118804 | | Down-reg |
| ZEB2 | ENSG00000169554 | | Down-reg |
| RAB7B | ENSG00000276600 | | Down-reg |
| DDIT3 | ENSG00000175197 | | Down-reg |
| CTH | ENSG00000116761 | | Down-reg |
| CARS1 | ENSG00000110619 | | Down-reg |
| ILDR2 | ENSG00000143195 | | Down-reg |
| ANGPTL6 | ENSG00000130812 | | Down-reg |
| ABHD14A | ENSG00000248487 | | Down-reg |
| MTHFD2 | ENSG00000065911 | | Down-reg |
| P2RX3 | ENSG00000109991 | | Down-reg |
| GPR141 | ENSG00000187037 | | Down-reg |
| ATF5 | ENSG00000169136 | | Down-reg |
| ALDH18A1 | ENSG00000059573 | | Down-reg |
| PYCR1 | ENSG00000183010 | | Down-reg |
| SNHG12 | ENSG00000197989 | | Down-reg |
| CD68 | ENSG00000129226 | | Down-reg |
| TMEM50B | ENSG00000142188 | | Up-reg |
| URAD | ENSG00000183463 | | Up-reg |
| CST9L | ENSG00000101435 | | Up-reg |
| FLRT3 | ENSG00000125848 | Secretome | Up-reg |
| MCF2L | ENSG00000126217 | | Up-reg |
| FAM3B | ENSG00000183844 | Secretome | Up-reg |
| SLC2A10 | ENSG00000197496 | | Up-reg |
| OLFM4 | ENSG00000102837 | Secretome | Up-reg |
| HAO1 | ENSG00000101323 | | Up-reg |
| IFNGR2 | ENSG00000159128 | | Up-reg |
| CYP2C18 | ENSG00000108242 | | Up-reg |
| GPD1 | ENSG00000167588 | | Up-reg |
| DEPP1 | ENSG00000165507 | | Up-reg |
| DDC | ENSG00000132437 | | Up-reg |
| SLC39A9 | ENSG00000029364 | | Up-reg |
| CYP2D7 | ENSG00000205702 | | Up-reg |
| MX1 | ENSG00000157601 | | Up-reg |
| AMBP | ENSG00000106927 | Secretome | Up-reg |
| SMIM24 | ENSG00000095932 | | Up-reg |
| IL13RA2 | ENSG00000123496 | | Up-reg |
| DMKN | ENSG00000161249 | Secretome | Up-reg |
| CLU | ENSG00000120885 | Secretome | Up-reg |
| TFF3 | ENSG00000160180 | | Up-reg |
| SLC18A1 | ENSG00000036565 | | Up-reg |
| WDR1 | ENSG00000071127 | | Up-reg |
| TMPRSS6 | ENSG00000187045 | | Up-reg |
| DHRS3 | ENSG00000162496 | | Up-reg |
| BCL2L14 | ENSG00000121380 | | Up-reg |
| LDLRAD3 | ENSG00000179241 | | Up-reg |
| IGFBP5 | ENSG00000115461 | Secretome | Up-reg |
| ALDOB | ENSG00000136872 | | Up-reg |
| FABP1 | ENSG00000163586 | | Up-reg |
| SCAMP1 | ENSG00000085365 | | Up-reg |
| HADHB | ENSG00000138029 | | Up-reg |
| FAM3D | ENSG00000198643 | Secretome | Up-reg |
| CLCA1 | ENSG00000016490 | Secretome | Up-reg |
| UQCRC2 | ENSG00000140740 | | Up-reg |
| TLR3 | ENSG00000164342 | | Up-reg |
| PSCA | ENSG00000167653 | | Up-reg |
| CLDN2 | ENSG00000165376 | | Up-reg |
| PIWIL4 | ENSG00000134627 | | Up-reg |
| ACE2 | ENSG00000130234 | | Up-reg |
| MUC20 | ENSG00000176945 | | Up-reg |
| SLC44A3 | ENSG00000143036 | | Up-reg |
| FRK | ENSG00000111816 | | Up-reg |
| SPP2 | ENSG00000072080 | | Up-reg |
| DMBT1 | ENSG00000187908 | | Up-reg |
| PLA2G10 | ENSG00000069764 | | Up-reg |
| ATP7A | ENSG00000165240 | | Up-reg |
| GALNT17 | ENSG00000185274 | | Up-reg |
| ASB13 | ENSG00000196372 | | Up-reg |
| KRT7 | ENSG00000135480 | | Up-reg |
| ANXA13 | ENSG00000104537 | | Up-reg |
| CKMT1B | ENSG00000237289 | | Up-reg |
| CKMT1A | ENSG00000223572 | | Up-reg |
| FMR1 | ENSG00000102081 | | Up-reg |
| ATP1A3 | ENSG00000105409 | | Up-reg |
| SOBP | ENSG00000112320 | | Up-reg |
| NAALADL2 | ENSG00000177694 | | Up-reg |
| KCNK16 | ENSG00000095981 | | Up-reg |
| CYP2D6 | ENSG00000100197 | | Up-reg |
| EPS8L1 | ENSG00000131037 | | Up-reg |
| F5 | ENSG00000198734 | | Up-reg |
| UGT1A6 | ENSG00000167165 | | Up-reg |
| KRT20 | ENSG00000171431 | | Up-reg |
| CDH16 | ENSG00000166589 | | Up-reg |
| PGC | ENSG00000096088 | Secretome | Up-reg |
| ANO7 | ENSG00000146205 | | Up-reg |
| USH1C | ENSG00000006611 | | Up-reg |
| TMPRSS4 | ENSG00000137648 | | Up-reg |
| UGT1A10 | ENSG00000242515 | | Up-reg |
| UGT1A9 | ENSG00000241119 | | Up-reg |
| UGT1A8 | ENSG00000242366 | | Up-reg |
| UGT1A7 | ENSG00000244122 | | Up-reg |
| CD55 | ENSG00000196352 | Secretome | Up-reg |
| IL5RA | ENSG00000091181 | | Up-reg |
| CXCL17 | ENSG00000189377 | Secretome | Up-reg |
| GKN2 | ENSG00000183607 | | Up-reg |
| TMC4 | ENSG00000167608 | | Up-reg |
| CTSE | ENSG00000196188 | | Up-reg |
| ABCB9 | ENSG00000150967 | | Up-reg |
| CYP4B1 | ENSG00000142973 | | Up-reg |
| SLC9A4 | ENSG00000180251 | | Up-reg |
| CHST4 | ENSG00000140835 | | Up-reg |
| OTOP3 | ENSG00000182938 | | Up-reg |
| LIPA | ENSG00000107798 | | Up-reg |
| MUC1 | ENSG00000185499 | | Up-reg |
| CD38 | ENSG00000004468 | | Up-reg |
| HMGCS2 | ENSG00000134240 | | Up-reg |
| ABCC8 | ENSG00000006071 | | Up-reg |
| RBP2 | ENSG00000114113 | | Up-reg |
| GIMAP8 | ENSG00000171115 | | Up-reg |
| EHF | ENSG00000135373 | | Up-reg |
| STAB2 | ENSG00000136011 | | Up-reg |
| TMEM236 | ENSG00000148483 | | Up-reg |
| C2orf72 | ENSG00000204128 | | Up-reg |
| ACSM3 | ENSG00000005187 | | Up-reg |
| SGK1 | ENSG00000118515 | | Up-reg |
| FXYD3 | ENSG00000089356 | | Up-reg |
| VIL1 | ENSG00000127831 | | Up-reg |
| ADGRG7 | ENSG00000144820 | | Up-reg |
| ABCG8 | ENSG00000143921 | | Up-reg |
| MUC3A | ENSG00000169894 | | Up-reg |
| SECTM1 | ENSG00000141574 | Secretome | Up-reg |
| S100A14 | ENSG00000189334 | | Up-reg |
| PYURF | ENSG00000145337 | | Up-reg |
| HP | ENSG00000257017 | Secretome | Up-reg |
| HPR | ENSG00000261701 | | Up-reg |
| GPA33 | ENSG00000143167 | | Up-reg |
| FOXJ1 | ENSG00000129654 | | Up-reg |
| AQP1 | ENSG00000240583 | | Up-reg |
| SPTBN2 | ENSG00000173898 | | Up-reg |
| TM4SF20 | ENSG00000168955 | | Up-reg |
| CES3 | ENSG00000172828 | | Up-reg |
| KRT23 | ENSG00000108244 | | Up-reg |
| PIGR | ENSG00000162896 | | Up-reg |
| APOA1 | ENSG00000118137 | Secretome | Up-reg |
| SLFN12 | ENSG00000172123 | | Up-reg |
| TRPM8 | ENSG00000144481 | | Up-reg |
| CLCN2 | ENSG00000114859 | | Up-reg |
| EPHA1 | ENSG00000146904 | | Up-reg |
| KIF12 | ENSG00000136883 | | Up-reg |
| PDZK1IP1 | ENSG00000162366 | | Up-reg |
| PHGR1 | ENSG00000233041 | | Up-reg |
| PILRA | ENSG00000085514 | Secretome | Up-reg |
| PZP | ENSG00000126838 | Secretome | Up-reg |
| TTYH1 | ENSG00000167614 | | Up-reg |
| SYCN | ENSG00000179751 | | Up-reg |
| SULT1A1 | ENSG00000196502 | | Up-reg |
| H19 | ENSG00000130600 | | Up-reg |
| MUC4 | ENSG00000145113 | Secretome | Up-reg |

* Secretome refers to the set of proteins that are differentially secreted by cancer cells with high or low FMRP pathway activity that can for example be used as biomarkers in liquid biopsy assays and other diagnostic bioassays.
“Up-reg indicates that a gene is positively regulated by FMRP-activity and Down-reg conversely indicates that a gene is negatively regulated by FMRP-activity.”

As used herein, MRC1 is: mannose receptor C-type 1; KDELR3 is: KDEL endoplasmic reticulum protein retention receptor 3; SLC7A1 is: solute carrier family 7 member 1; PIK3CD is: phosphatidylinositol-4,5-bisphosphate 3-kinase catalytic subunit delta; BCAT1 is: branched chain amino acid transaminase 1; JDP2 is: Jun dimerization protein 2; ADGRA2 is: adhesion G protein-coupled receptor A2; HMOX1 is: heme oxygenase 1; COBL is: cordon-bleu WH2 repeat protein; PSAT1 is: phosphoserine aminotransferase 1; CHD5 is: chromodomain helicase DNA binding protein 5; CHAC1 is: ChaC glutathione specific gamma-glutamylcyclotransferase 1; ATP2A3 is: ATPase sarcoplasmic/endoplasmic reticulum Ca2+transporting 3; EIF4EBP1 is:eukaryotic translation initiation factor 4E binding protein 1; CA6 is: carbonic anhydrase 6; AVIL is: advillin; PSPH is: phosphoserine phosphatase; HMGA1 is: high mobility group AT-hook 1; ATF4 is: activating transcription factor 4; SLC1A4 is: solute carrier family 1 member 4; CIART is: circadian associated repressor of transcription; TRIB3 is: tribbles pseudokinase 3; LIMS4 is: LIM zinc finger domain containing 4; AREG is: amphiregulin; IFRD1 is: interferon related developmental regulator 1; SLC7A11 is: solute carrier family 7 member 11; ASNS is: asparagine synthetase (glutamine-hydrolyzing); ACAT2 is: acetyl-CoA acetyltransferase 2; LHFPL2 is: LHFPL tetraspan subfamily member 2; EXTL1 is: exostosin like glycosyltransferase 1; FOSL1 is: FOS like 1, AP-1 transcription factor subunit; CDSN is: corneodesmosin; SNAI2 is: snail family transcriptional repressor 2; ALDH1L2 is: aldehyde dehydrogenase 1 family member L2; SLC7A5 is: solute carrier family 7 member 5; TMEM266 is: transmembrane protein 266; PCK2 is: phosphoenolpyruvate carboxykinase 2, mitochondrial; PHF19 is: PHD finger protein 19; FTL is: ferritin light chain; GRAMD2A is: GRAM domain containing 2A; CPS1 is: carbamoyl-phosphate synthase 1; CAV1 is: caveolin 1; UNC13C is: unc-13 homolog C; BEND6 is: BEN domain containing 6; TIGIT is: T cell immunoreceptor with Ig and ITIM domains; YARS1 is: tyrosyl-tRNA synthetase 1; LIMS3 is: LIM zinc finger domain containing 3; STBD1 is: starch binding domain 1; ZEB2 is: zinc finger E-box binding homeobox 2; RAB7B is: RAB7B, member RAS oncogene family; DDIT3 is: DNA damage inducible transcript 3; CTH is: cystathionine gamma-lyase; CARS1 is: cysteinyl-tRNA synthetase 1; ILDR2 is: immunoglobulin like domain containing receptor 2; ANGPTL6 is: angiopoietin like 6; ABHD14A is: abhydrolase domain containing 14A; MTHFD2 is: methylenetetrahydrofolate dehydrogenase (NADP+dependent) 2, methenyltetrahydrofolate cyclohydrolase; P2RX3 is: purinergic receptor P2X 3; GPR141 is: G protein-coupled receptor 141; ATF5 is: activating transcription factor 5; ALDH18A1 is: aldehyde dehydrogenase 18 family member A1; PYCR1 is: pyrroline-5-carboxylate reductase 1; SNHG12 is: small nucleolar RNA host gene 12; CD68 is: CD68 molecule; TMEMSOB is: transmembrane protein 50B; URAD is: ureidoimidazoline (2-oxo-4-hydroxy-4-carboxy-5-) decarboxylase; CST9L is: cystatin 9 like; FLRT3 is: fibronectin leucine rich transmembrane protein 3; MCF2L is: MCF.2 cell line derived transforming sequence like; FAM3B is: FAM3 metabolism regulating signaling molecule B; SLC2A10 is: solute carrier family 2 member 10; OLFM4 is: olfactomedin 4; HAO1 is: hydroxyacid oxidase 1; IFNGR2 is: interferon gamma receptor 2; CYP2C18 is: cytochrome P450 family 2 subfamily C member 18; GPD1 is: glycerol-3-phosphate dehydrogenase 1; DEPP1 is: DEPP1 autophagy regulator; DDC is: dopa decarboxylase; SLC39A9 is: solute carrier family 39 member 9; CYP2D7 is: cytochrome P450 family 2 subfamily D member 7 (gene/pseudogene); MX1 is: MX dynamin like GTPase 1; AMBP is: alpha-1-microglobulin/bikunin precursor; SMIM24 is: small integral membrane protein 24; IL13RA2 is: interleukin 13 receptor subunit alpha 2; DMKN is: dermokine; CLU is: clusterin; TFF3 is: trefoil factor 3; SLC18A1 is: solute carrier family 18 member A1; WDR1 is: WD repeat domain 1; TMPRSS6 is: transmembrane serine protease 6; DHRS3 is: dehydrogenase/reductase 3; BCL2L14 is: BCL2 like 14; LDLRAD3 is: low density lipoprotein receptor class A domain containing 3; IGFBP5 is: insulin like growth factor binding protein 5; ALDOB is: aldolase, fructose-bisphosphate B; FABP1 is: fatty acid binding protein 1; SCAMPI is: secretory carrier membrane protein 1; HADHB is: hydroxyacyl-CoA dehydrogenase trifunctional multienzyme complex subunit beta; FAM3D is: FAM3 metabolism regulating signaling molecule D; CLCA1 is: chloride channel accessory 1; UQCRC2 is: ubiquinol-cytochrome c reductase core protein 2; TLR3 is: toll like receptor 3; PSCA is: prostate stem cell antigen; CLDN2 is: claudin 2; PIWIL4 is: piwi like RNA-mediated gene silencing 4; ACE2 is: angiotensin converting enzyme 2; MUC20 is: mucin 20, cell surface associated; SLC44A3 is: solute carrier family 44 member 3; FRK is: fyn related Src family tyrosine kinase; SPP2 is: secreted phosphoprotein 2; DMBT1 is: deleted in malignant brain tumors 1; PLA2G10 is: phospholipase A2 group X; ATP7A is: ATPase copper transporting alpha; GALNT17 is: polypeptide N-acetylgalactosaminyltransferase 17; ASB13 is: ankyrin repeat and SOCS box containing 13; KRT7 is: keratin 7; ANXA13 is: annexin A13; CKMT1B is: creatine kinase, mitochondrial 1B; CKMT1A is: creatine kinase, mitochondrial 1A; FMR1 is: FMRP translational regulator 1; ATP1A3 is: ATPase Na+/K+transporting subunit alpha 3; SOBP is: sine oculis binding protein homolog; NAALADL2 is: N-acetylated alpha-linked acidic dipeptidase like 2; KCNK16 is: potassium two pore domain channel subfamily K member 16; CYP2D6 is: cytochrome P450 family 2 subfamily D member 6; EPS8L1 is: EPS8 like 1; F5 is: coagulation factor V; UGT1A6 is: UDP glucuronosyltransferase family 1 member A6; KRT20 is: keratin 20; CDH16 is: cadherin 16; PGC is: progastricsin; ANO7 is: anoctamin 7; USH1C is: USH1 protein network component harmonin; TMPRSS4 is: transmembrane serine protease 4; UGT1A10 is: UDP glucuronosyltransferase family 1 member A10; UGT1A9 is: UDP glucuronosyltransferase family 1 member A9; UGT1A8 is: UDP glucuronosyltransferase family 1 member A8; UGT1A7 is: UDP glucuronosyltransferase family 1 member A7; CD55 is: CD55 molecule (Cromer blood group); IL5RA is: interleukin 5 receptor subunit alpha; CXCL17 is: C-X-C motif chemokine ligand 17; GKN2 is: gastrokine 2; TMC4 is: transmembrane channel like 4; CTSE is: cathepsin E; ABCB9 is: ATP binding cassette subfamily B member 9; CYP4B1 is: cytochrome P450 family 4 subfamily B member 1; SLC9A4 is: solute carrier family 9 member A4; CHST4 is: carbohydrate sulfotransferase 4; OTOP3 is: otopetrin 3; LIPA is: lipase A, lysosomal acid type; MUC1 is: mucin 1, cell surface associated; CD38 is: CD38 molecule; HMGCS2 is: 3-hydroxy-3-methylglutaryl-CoA synthase 2; ABCC8 is: ATP binding cassette subfamily C member 8; RBP2 is: retinol binding protein 2; GIMAP8 is: GTPase, IMAP family member 8; EHF is: ETS homologous factor; STAB2 is: stabilin 2; TMEM236 is: transmembrane protein 236; C2orf72 is: chromosome 2 open reading frame 72; ACSM3 is: acyl-CoA synthetase medium chain family member 3; SGK1 is: serum/glucocorticoid regulated kinase 1; FXYD3 is: FXYD domain containing ion transport regulator 3; VIL1 is: villin 1; ADGRG7 is: adhesion G protein-coupled receptor G7; ABCG8 is: ATP binding cassette subfamily G member 8; MUC3A is: mucin 3A, cell surface associated; SECTM1 is: secreted and transmembrane 1; S100A14 is: S100 calcium binding protein A14; PYURF is: PIGY upstream open reading frame; HP is: haptoglobin; HPR is: haptoglobin-related protein; GPA33 is: glycoprotein A33; FOXJ1 is: forkhead box J1; AQP1 is: aquaporin 1 (Colton blood group); SPTBN2 is: spectrin beta, non-erythrocytic 2; TM4SF20 is: transmembrane 4 L six family member 20; CES3 is: carboxylesterase 3; KRT23 is: keratin 23; PIGR is: polymeric immunoglobulin receptor; APOA1 is: apolipoprotein A1; SLFN12 is: schlafen family member 12; TRPM8 is: transient receptor potential cation channel subfamily M member 8; CLCN2 is: chloride voltage-gated channel 2; EPHA1 is: EPH receptor A1; KIF12 is: kinesin family member 12; PDZKlIP1 is: PDZK1 interacting protein 1; PHGR1 is: proline, histidine and glycine rich 1; PILRA is: paired immunoglobin like type 2 receptor alpha; PZP is: PZP alpha-2-macroglobulin like; TTYH1 is: tweety family member 1; SYCN is: syncollin; SULT1A1 is: sulfotransferase family 1A member 1; H19 is: H19 imprinted maternally expressed transcript; MUC4 is: mucin 4, cell surface associated.

As used herein, the Pan-Signature list, the Sub-Signature lists (Sub-Signatures 1, 2, and/or 3), the cancer type-specific lists, and Pan-Immunosuppressive signature list are individually and collectively referred to herein as “signature(s) of the invention”.

The present invention relates to the identification and use of gene expression patterns (or profiles or signatures), which are clinically relevant to cancer therapy. In particular, the invention identifies genes that are correlated with the evaluation, treatment and monitoring of patients for cancer treatment.

The identified gene biomarkers embodied in the Pan-Signature list, the Sub-Signature lists, the cancer type-specific lists, and Pan-Immunosuppressive list constituting the invention do not involve or require assessment of FMR1 mRNA or FMRP protein expression, but rather independently predict the levels of signaling activity downstream of FMRP expression, wherein high levels of pathway activity in tumors predict the capability to suppress tumor immunity and/or to stimulate invasion and metastasis. The signatures described above can be the basis for multiplex biomarker assays to stratify cancer patients based on their FMRP activity, both to predict prognosis and inform treatment choices, and thus could serve as “companion diagnostics” for cancer therapy.

As used herein, a companion diagnostic refers to a diagnostic method and/or reagent that is used to identify patients susceptible to treatment with a particular treatment or to monitor treatment and/or to identify an effective dosage for a patient or a sub-group or other group of patients. The companion diagnostic refers to the reagents and also to the test(s) that is/are performed with the reagent.

As used herein, a “patient”, “subject” and “individual” are used interchangeably and refer to a human subject having cancer or exhibiting symptoms of cancer.

In embodiments, the invention provides a method for identifying a patient with cancer as being high or low for FMRP activity having a high or low risk prognosis and/or being a responder or non-responder to cancer therapy. In embodiments, the method comprises obtaining a sample from the patient; determining an expression level for the genes in one or more signatures set forth in Tables 1 through 33 in the sample; comparing the expression levels in the sample relative to the level of said genes expressed in a control; and identifying the differentially expressed gene(s) between the sample and control; and classifying the patient as (i) being high or low for FMRP activity, (ii) having a high or low risk prognosis and/or (iii) being a responder or non-responder to cancer therapy, and/or (iv) having high or low immune cell infiltrated tumor, based on the concordance of the differential expression with the one or more signatures.

As used in any of the embodiments herein, the term “control”, or the like, refers one or more samples which has known FMRP activity status and/or clinical information. Therefore, relative to this control, the FMRP activity in a patient sample (the query sample(s)), is determined, and accordingly, the clinical outcome (prognosis or response to a cancer therapy) is predicted. Control can be of the same or different constitutions than the patient sample, including but not limited to: one or more tumor samples from the same cancer type which has known prognosis and/or response to a form of therapy; or a cohort of samples from publicly available datasets (e.g. TCGA) profiling tumor samples that have a variety of FMRP activities; additionally, cognate normal samples in some cases can serve as the control cohort, depending on the tissue and the activity of FMRP in normal cells. For example, if a patient has breast cancer, the control can be a set(s) of previously analyzed tumor samples from a cohort of breast cancer patients amongst whom some have high and others low FMRP activity scores, potentially embellished with additional clinical or pathological information. This cohort can be used as a reference set to establish a high vs. low FMRP activity score for the new tumor being queried and the particular prognostic/therapeutic question being addressed. Alternatively, for example, a TCGA cohort of breast cancer tumors that can be segregated into groups with high, neutral, or low FMRP-activity scores, and can be used as a reference in order to classify the tumor being queried for its FMRP-activity.

For an FMRP-activity signature to have predictive power, at least one (1), or at least two (2), or at least ten (10) genes from the PAN-Signature list, and/or from a Sub-Signature or from a cancer type-specific signature list thereof, should be differentially expressed between the patient sample and the control. If this criterion is met, the query sample is then classified as follows. If a super-majority of the differentially expressed genes follows the expected up-/down-regulated calls within the signature list—i.e., differentially up-regulated genes in the sample are in the signature list of up-regulated genes, and differentially down-regulated genes in the sample are also in the signature list of down-regulated genes—then the query sample has higher FMRP-activity compared to the control. Conversely, if the super-majority of the differentially expressed genes show an opposite pattern within the signature list—i.e., differentially up-regulated genes in the sample are part of the signature list of ostensibly down-regulated genes, and differentially down-regulated genes in the sample come from the signature list of up-regulated genes—then the query sample is judged to have a lower FMRP-activity compared to the control. As used herein, the phrase “a super-majority of the differentially expressed genes” generally means that ⅔ of the differentially expressed genes in the sample follow or do not follow the regulated calls (i.e., up-/down-regulated) within the signature list.

As illustrated in the Figures herein, with respect to other patients with the same cancer type or subtype:
low FMRP activity signature score is associated with better prognosis;
low FMRP activity signature score is associated with a patient being a comparatively better responder to treatment with a checkpoint inhibitor, targeted cancer therapy, chemotherapy, or radiation, but being a non-responder or a less robust responder to treatment with a FMRP inhibitor; and
high FMRP activity signature score is associated with a patient being a comparatively better responder to treatment with a FMRP inhibitor but a non-responder or a comparatively poor responder to treatment with a checkpoint inhibitor, targeted cancer therapy, chemotherapy, or radiation unless combined with an FMRP inhibitor.
low Pan-Immunosuppression signature score is associated with comparatively higher inflamed tumors by T cells.

As used in any of the methods described herein, the terms “differentially expressed” or “altered expression” are used interchangeably to refer to a difference in the level of expression of the RNA of the biomarkers of the invention, as measured by the amount or level of mRNA, and/or one or more spliced variants of mRNA of the biomarker in one sample as compared with the level of expression of the same biomarker of the invention in a second sample. “Differentially expressed” or “altered expression” can also include a measurement of the protein encoded by a biomarker of the invention in a sample or population of samples as compared with the amount or level of protein expression in a second sample or population of samples. Differential expression can be determined as described herein and as would be understood by a person skilled in the art. A gene or protein is either upregulated or down regulated in a cancer patient as compared to a control. A gene is considered either upregulated or downregulated if its expression in the patient sample is increased or decreased at least 1.5-fold as compared to its expression level in a corresponding control. For purposes herein, the altered expression of a gene is a result of FMRP functional activity in tumors.

As used in any of the embodiments herein, the phrase “relative to levels of said genes expressed in control”, or the like, refers to the expression level of the genes on the invention in control samples, depending on each specific study, as described herein.

In embodiments, the invention provides a method for identifying a patient with cancer as eligible for cancer therapy. In embodiments, the method comprises obtaining a sample from the patient; determining expression levels in the sample of the genes in one or more of the signatures set forth in Tables 1 through 33; comparing the levels of expression relative to levels of said genes expressed in a corresponding control; and identifying the patient as eligible to receive a cancer therapy based on the concordance of the differential expression with the signatures.

In embodiments, the invention provides a method for identifying a patient with cancer as a responder to cancer therapy. In embodiments, the method comprises obtaining a sample from the patient; determining expression levels in the sample of the genes in one or more signatures set forth in Tables 1 through 33; comparing the levels of expression relative to levels of said genes expressed in a corresponding control; and identifying the patient as responder to cancer therapy based on the concordance of the differential expression with the signatures.

In embodiments, the invention provides a method for treating a patient with cancer. In embodiments, the method comprises obtaining a sample from the patient; determining expression level for the genes in one or more of the signatures set forth in Tables 1 through 33 in the sample; comparing the expression levels in the sample relative to the level of said genes expressed in a control; identifying the differentially expressed genes between the sample and control; classifying the patient as (i) being high or low for FMRP activity, (ii) having a high or low risk prognosis and/or (iii) being a responder or non-responder to cancer therapy, and/or (iv) having high or low immune cell infiltrated tumor, based on the concordance of the differential expression with the signatures; and administering a cancer therapy to the patient.

In any of the embodiments herein, the method comprises determining an expression level for the genes in one signature set forth in Tables 1 through 33. In any of the embodiments herein, the method comprises determining an expression level for the genes in two or more signatures set forth in Tables 1 through 33.

In any of the embodiments herein, the method comprises determining an expression level for each gene in the Pan-Signature set forth in Table 1. In any of the embodiments herein, the method comprises determining an expression level for the genes in one or more signatures set forth in Tables 1-33.

In any of the embodiments herein, the method comprises determining an expression level for each gene in one or more Sub-Signatures and/or cancer type-specific signatures as set forth in Tables 2-33 in the tissue sample; and comparing these expression levels relative to the level of said genes expressed in a control. In any of the embodiments herein, the method comprises determining an expression level for the genes in one or more Sub-Signatures as set forth in Tables 2-4. In any of the embodiments herein, the method comprises determining an expression level for the genes in one or more cancer specific signatures as set forth in Tables 5-32.

In any of the embodiments herein, the method comprises determining an expression level for the genes in the Pan-Immunosuppressive Signature as set forth in Table 33.

The invention also provides a method for developing a signature score as a biomarker of FMRP-activity in a group of patients with cancer. In some embodiments, where there is a specific set of samples from cancer patients being analyzed without a separate reference set, for example a group involving a distinctive histologic or molecular subtype of a particular cancer type, or with variable responses (tumor size, PSF, OS) to a particular therapy, then the signature score can be derived for each sample relative to all other samples in the group.

In embodiments, the invention provides a method for stratifying a group of patients with cancer as (i) being high or low for FMRP activity, (ii) having a high or low risk prognosis and/or (iii) being a responder or non-responder to cancer therapy and/or (iv) having high or low immune cell infiltrated tumor. In embodiments, the method comprises obtaining a sample from each patient of the group; determining expression level of the genes in one or more of the signatures set forth in Tables 1 through 33 for each sample; establishing an FMRP activity score for each sample; and identifying each patient as (i) being high or low for FMRP activity, (ii) having a high or low risk prognosis and/or (iii) being a responder or non-responder to cancer therapy, and/or (iv) having high or low immune cell infiltrated tumor based on the FMRP activity score.

In embodiments, the invention provides a method for stratifying a group of patients with cancer as eligible for cancer therapy. In embodiments, the method comprises obtaining a sample from each patient of the group; determining expression level of the genes in one or more of the signatures set forth in Tables 1 through 33 for each sample; establishing an FMRP activity score for each sample; and identifying the patient as eligible to receive a cancer therapy based on the FMRP activity score.

In embodiments, the invention provides a method for stratifying a group of patients with cancer as a responder to cancer therapy. In embodiments, the method comprises obtaining a sample from each patient of the group; determining expression level of the genes in one or more of the signatures set forth in Tables 1 through 33 for each sample; establishing an FMRP activity score for each sample; and identifying the patient as eligible to receive a cancer therapy based on the FMRP activity score.

In embodiments, the invention provides a method for treating a group of patients with cancer. In embodiments, the method comprises obtaining a sample from each patient of the group; determining expression level of the genes in one or more of the signatures set forth in Tables 1 through 33 for each sample; establishing an FMRP activity score for each sample; identifying each patient as (i) being high or low for FMRP activity, (ii) having a high or low risk prognosis and/or (iii) being a responder or non-responder to cancer therapy, and/or (iv) having high or low immune cell infiltrated tumor based on the FMRP activity score; and administering a cancer therapy to each patient.

In embodiments, the invention provides a method for predicting T-cell infiltration in a cancer patient. In embodiments, the method comprises obtaining a sample from the patient; determining expression level for the genes set forth in Table 33 in the sample; comparing the expression levels in step (b) relative to the level of said genes expressed in a control; identifying the differentially expressed gene(s) between the sample and control; and classifying the patient as (i) being high or low for FMRP immunosuppressive activity, (ii) having a high or low immune cell infiltration based on the concordance of the differential expression with the signature.

As used in this embodiment, the term “signature score”, also referred to herein as the “FMRP-activity signature score”, generally refers to a quantitative score which predicts whether a patient will benefit from currently available cancer therapies that are limited in efficacy or otherwise dependent on FMRP activity, or are potentially modulated by FMRP. A signature score is calculated by summing the z-score of the genes within a particular FMRP-activity signature list (e.g., PAN-Signature and/or a Sub-Signature and/or a cancer specific signature and/or Pan-Immunosuppressive signature thereof), for example, the number of standard deviations by which the expression is above or below the mean value of expressions for the gene in all samples. For down-regulated genes in a signature, the z-scores are multiplied by minus one (−1) before summing up to derive the final signature score.

In any of the methods described herein, according to the signature scores, the cancer patients with low FMRP-activity scores are expected to have a better prognosis and a better response to cancer therapies compared to cancer patients with high FMRP-activity score. Cancer patients with a high FMRP-activity score are expected to have a better response to treatment with an FMRP inhibitor.

In some embodiments, the predictive power of the FMRP-activity signature score in such a group can, optionally, be confirmed if at least one (1), or at least two (2), or at least ten (10) genes from the signature list are differentially expressed between the top 50% of the samples with respect to signature score (samples having signature scores higher than the median) and lower 50% of the samples with respect to signature score (samples having signature scores smaller than the median). If this criterion is met, the samples with low FMRP-activity signature scores (samples having signature scores smaller than the median or 1^{st} quartile) have better prognosis, or better response to cancer therapy, whereas samples with high FMRP-activity signature scores (samples having signature scores larger than the median or 1st quartile) have worse prognosis, or poor/no response to a cancer therapy, or potentially have a better response to treatment with an FMRP inhibitor.

The invention provides companion diagnostic assays for classification of patients for cancer treatment which comprise assessment in a patient tissue sample the levels of expression of genes set out in TABLES 1 through 33, or combinations thereof. The inventive assays include assay methods for identifying patients eligible to receive cancer therapy and for monitoring patient response to such therapy. The invention methods comprise assessment of the expression of said genes in blood, urine, or other body fluid samples by immunoassay, proteomic assay or nucleic acid hybridization or amplification or sequencing assays, and in tissue or other cellular body samples by immunohistochemistry or in situ hybridization assays.

Gene expression patterns of the invention, also referred to as “gene expression pattern” or “gene expression profile” or “gene signature”, are identified as described herein. Generally, the gene expression profile of a sample is obtained through quantifying the expression levels of mRNA corresponding to many genes identified in the signature lists of Tables 1 through 33. The signature is then analyzed to identify genes, the expression of which are positively correlated with the identification of and monitoring of patients eligible of cancer treatment.

In any of the embodiments herein, the gene signature indicates the combined pattern of the results of the analysis of the level of expression of one or more genes of the signatures of the invention. In embodiments, the gene signature is the result of the analysis of the level of expression of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, or all of the genes of the signatures of the invention.

In any of the embodiments herein, the gene signature indicates the combined pattern of the results of the analysis of the level of expression of one or more genes of one or more signatures of the invention. In embodiments, the gene signature is the result of the analysis of the level of expression of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, or all of the genes of one or more signatures of the invention.

In any of the embodiments herein, a gene signature indicates the combined pattern of the results of the analysis of the level of expression of ten or more genes of the signatures of the invention. In embodiments, the gene signature is the result of the analysis of the level of expression of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, or all of the genes of the signatures of the invention.

In any of the embodiments herein, a gene signature indicates the combined pattern of the results of the analysis of the level of expression of ten or more genes of one or more signatures of the invention. In embodiments, the gene signature is the result of the analysis of the level of expression of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, or all of the genes of one or more signatures of the invention.

In any embodiments of the invention, the minimum number of genes (biomarkers) that are required for the use of the signature lists of the invention to improve identification or stratification of patents is at least 1 the biomarkers of the signatures of the invention. In any embodiments of the invention, the minimum number of genes that are required for the use of the signature lists of the invention is at least 2 the biomarkers of the signatures of the invention. In any embodiments of the invention, the minimum number of genes that are required for the use of the signature lists of the invention is at least 10 the biomarkers of the signatures of the invention. In any embodiments of the invention, the minimum number of genes (biomarkers) that are required for the use of the signature lists of the invention is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, or all of the biomarkers of the signatures of the invention.

In any embodiments of the invention, the minimum number of genes (biomarkers) that are required for the use of the signature lists of the invention to improve identification or stratification of patents is at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, or all of the biomarkers of the signatures of the invention.

In any embodiments of the invention, biomarkers to improve identification or stratification of patients comprises at least 1 of the biomarkers in TABLE 1, TABLE 2, TABLE 3, TABLE 4, TABLE 5, TABLE 6, TABLE 7, TABLE 8, TABLE 9, TABLE 10, TABLE 11, TABLE 12, TABLE 13, TABLE 14, TABLE 15, TABLE 16, TABLE 17, TABLE 18, TABLE 19, TABLE 20, TABLE 21, TABLE 22, TABLE 23, TABLE 24, TABLE 25, TABLE 26, TABLE 27, TABLE 28, TABLE 29, TABLE 30, TABLE 31, TABLE 32, or combinations thereof. In another embodiment, biomarkers to improve identification or stratification of patients comprises at least 1 of the biomarkers in TABLE 1 and/or TABLE 2, TABLE 3, TABLE 4, TABLE 5, TABLE 6, TABLE 7, TABLE 8, TABLE 9, TABLE 10, TABLE 11, TABLE 12, TABLE 13, TABLE 14, TABLE 15, TABLE 16, TABLE 17, TABLE 18, TABLE 19, TABLE 20, TABLE 21, TABLE 22, TABLE 23, TABLE 24, TABLE 25, TABLE 26, TABLE 27, TABLE 28, TABLE 29, TABLE 30, TABLE 31, TABLE 32. In another embodiment, biomarkers to improve identification or stratification of patients comprises at least 1 of the biomarkers in TABLE 1 and one or more of TABLE 2, TABLE 3, TABLE 4, TABLE 5, TABLE 6, TABLE 7, TABLE 8, TABLE 9, TABLE 10, TABLE 11, TABLE 12, TABLE 13, TABLE 14, TABLE 15, TABLE 16, TABLE 17, TABLE 18, TABLE 19, TABLE 20, TABLE 21, TABLE 22, TABLE 23, TABLE 24, TABLE 25, TABLE 26, TABLE 27, TABLE 28, TABLE 29, TABLE 30, TABLE 31, and/or TABLE 32.

In any embodiments of the invention, biomarkers to improve identification or stratification of patients comprises at least 1 of the biomarkers in TABLE 33.

In any embodiments of the invention, biomarkers to improve identification or stratification of patients comprises at least 2 of the biomarkers in TABLE 1, TABLE 2, TABLE 3, TABLE 4, TABLE 5, TABLE 6, TABLE 7, TABLE 8, TABLE 9, TABLE 10, TABLE 11, TABLE 12, TABLE 13, TABLE 14, TABLE 15, TABLE 16, TABLE 17, TABLE 18, TABLE 19, TABLE 20, TABLE 21, TABLE 22, TABLE 23, TABLE 24, TABLE 25, TABLE 26, TABLE 27, TABLE 28, TABLE 29, TABLE 30, TABLE 31, TABLE 32, or combinations thereof. In another embodiment, biomarkers to improve identification or stratification of patients comprises at least 2 of the biomarkers in TABLE 1 and/or TABLE 2, TABLE 3, TABLE 4, TABLE 5, TABLE 6, TABLE 7, TABLE 8, TABLE 9, TABLE 10, TABLE 11, TABLE 12, TABLE 13, TABLE 14, TABLE 15, TABLE 16, TABLE 17, TABLE 18, TABLE 19, TABLE 20, TABLE 21, TABLE 22, TABLE 23, TABLE 24, TABLE 25, TABLE 26, TABLE 27, TABLE 28, TABLE 29, TABLE 30, TABLE 31, TABLE 32. In another embodiment, biomarkers to improve identification or stratification of patients comprises at least 2 of the biomarkers in TABLE 1 and one or more of TABLE 2, TABLE 3, TABLE 4, TABLE 5, TABLE 6, TABLE 7, TABLE 8, TABLE 9, TABLE 10, TABLE 11, TABLE 12, TABLE 13, TABLE 14, TABLE 15, TABLE 16, TABLE 17, TABLE 18, TABLE 19, TABLE 20, TABLE 21, TABLE 22, TABLE 23, TABLE 24, TABLE 25, TABLE 26, TABLE 27, TABLE 28, TABLE 29, TABLE 30, TABLE 31, and/or TABLE 32.

In any embodiments of the invention, biomarkers to improve identification or stratification of patients comprises at least 2 of the biomarkers in TABLE 33.

In any embodiments of the invention, biomarkers to improve identification or stratification of patients comprises at least 10 of the biomarkers in TABLE 1, TABLE 2, TABLE 3, TABLE 4, TABLE 5, TABLE 6, TABLE 7, TABLE 8, TABLE 9, TABLE 10, TABLE 11, TABLE 12, TABLE 13, TABLE 14, TABLE 15, TABLE 16, TABLE 17, TABLE 18, TABLE 19, TABLE 20, TABLE 21, TABLE 22, TABLE 23, TABLE 24, TABLE 25, TABLE 26, TABLE 27, TABLE 28, TABLE 29, TABLE 30, TABLE 31, TABLE 32, or combinations thereof. In another embodiment, biomarkers to improve identification or stratification of patients comprises at least 10 of the biomarkers in TABLE 1 and/or TABLE 2, TABLE 3, TABLE 4, TABLE 5, TABLE 6, TABLE 7, TABLE 8, TABLE 9, TABLE 10, TABLE 11, TABLE 12, TABLE 13, TABLE 14, TABLE 15, TABLE 16, TABLE 17, TABLE 18, TABLE 19, TABLE 20, TABLE 21, TABLE 22, TABLE 23, TABLE 24, TABLE 25, TABLE 26, TABLE 27, TABLE 28, TABLE 29, TABLE 30, TABLE 31, TABLE 32. In another embodiment, biomarkers to improve identification or stratification of patients comprises at least 10 of the biomarkers in TABLE 1 and one or more of TABLE 2, TABLE 3, TABLE 4, TABLE 5, TABLE 6, TABLE 7, TABLE 8, TABLE 9, TABLE 10, TABLE 11, TABLE 12, TABLE 13, TABLE 14, TABLE 15, TABLE 16, TABLE 17, TABLE 18, TABLE 19, TABLE 20, TABLE 21, TABLE 22, TABLE 23, TABLE 24, TABLE 25, TABLE 26, TABLE 27, TABLE 28, TABLE 29, TABLE 30, TABLE 31, and/or TABLE 32.

In any embodiments of the invention, biomarkers to improve identification or stratification of patients comprises at least 10 of the biomarkers in TABLE 33.

In another embodiment, biomarkers to improve identification or stratification of patients comprises at least 1 of the biomarkers in TABLE 1. In another embodiment, biomarkers to improve identification or stratification of patients comprises at least 1 of the biomarkers in TABLE 2. In another embodiment, biomarkers to improve identification or stratification of patients comprises at least 1 of the biomarkers in in TABLE 3. In another embodiment, biomarkers to improve identification or stratification of patients comprises at least 1 of the biomarkers in TABLE 4. In another embodiment, biomarkers to improve identification or stratification of patients comprises at least 1 of the biomarkers in TABLE 5. In another embodiment, biomarkers to improve identification or stratification of patients comprises at least 1 of the biomarkers in TABLE 6. In another embodiment, biomarkers to improve identification or stratification of patients comprises at least 1 of the biomarkers in TABLE 7. In another embodiment, biomarkers to improve identification or stratification of patients comprises at least 1 of the biomarkers in TABLE 8. In another embodiment, biomarkers to improve identification or stratification of patients comprises at least 1 of the biomarkers in TABLE 9. In another embodiment, biomarkers to improve identification or stratification of patients comprises at least 1 of the biomarkers in TABLE 10. In another embodiment, biomarkers to improve identification or stratification of patients comprises at least 1 of the biomarkers in TABLE 11. In another embodiment, biomarkers to improve identification or stratification of patients comprises at least 1 of the biomarkers in TABLE 12. In another embodiment, biomarkers to improve identification or stratification of patients comprises at least 1 of the biomarkers in TABLE 13. In another embodiment, biomarkers to improve identification or stratification of patients comprises at least 1 of the biomarkers in TABLE 14. In another embodiment, biomarkers to improve identification or stratification of patients comprises at least 1 of the biomarkers in TABLE 15. In another embodiment, biomarkers to improve identification or stratification of patients comprises at least 1 of the biomarkers in TABLE 16. In another embodiment, biomarkers to improve identification or stratification of patients comprises at least 1 of the biomarkers in TABLE 17. In another embodiment, biomarkers to improve identification or stratification of patients comprises at least 1 of the biomarkers in TABLE 18. In another embodiment, biomarkers to improve identification or stratification of patients comprises at least 1 of the biomarkers in TABLE 19. In another embodiment, biomarkers to improve identification or stratification of patients comprises at least 1 of the biomarkers in TABLE 20. In another embodiment, biomarkers to improve identification or stratification of patients comprises at least 1 of the biomarkers in TABLE 21. In another embodiment, biomarkers to improve identification or stratification of patients comprises at least 1 of the biomarkers in TABLE 22. In another embodiment, biomarkers to improve identification or stratification of patients comprises at least 1 of the biomarkers in TABLE 23. In another embodiment, biomarkers to improve identification or stratification of patients comprises at least 1 of the biomarkers in TABLE 24. In another embodiment, biomarkers to improve identification or stratification of patients comprises at least 1 of the biomarkers in TABLE 25. In another embodiment, biomarkers to improve identification or stratification of patients comprises at least 1 of the biomarkers in TABLE 26. In another embodiment, biomarkers to improve identification or stratification of patients comprises at least 1 of the biomarkers in TABLE 27. In another embodiment, biomarkers to improve identification or stratification of patients comprises at least 1 of the biomarkers in TABLE 28. In another embodiment, biomarkers to improve identification or stratification of patients comprises at least 1 of the biomarkers in TABLE 29. In another embodiment, biomarkers to improve identification or stratification of patients comprises at least 1 of the biomarkers in TABLE 30. In another embodiment, biomarkers to improve identification or stratification of patients comprises at least 1 of the biomarkers in TABLE 31. In another embodiment, biomarkers to improve identification or stratification of patients comprises at least 1 of the biomarkers in TABLE 32.

In another embodiment, biomarkers to improve identification or stratification of patients comprises at least 1 of the biomarkers in TABLE 33.

In another embodiment, biomarkers to improve identification or stratification of patients comprises at least 2 of the biomarkers in TABLE 1. In another embodiment, biomarkers to improve identification or stratification of patients comprises at least 2 of the biomarkers in TABLE 2. In another embodiment, biomarkers to improve identification or stratification of patients comprises at least 2 of the biomarkers in in TABLE 3. In another embodiment, biomarkers to improve identification or stratification of patients comprises at least 2 of the biomarkers in TABLE 4. In another embodiment, biomarkers to improve identification or stratification of patients comprises at least 2 of the biomarkers in TABLE 5. In another embodiment, biomarkers to improve identification or stratification of patients comprises at least 2 of the biomarkers in TABLE 6. In another embodiment, biomarkers to improve identification or stratification of patients comprises at least 2 of the biomarkers in TABLE 7. In another embodiment, biomarkers to improve identification or stratification of patients comprises at least 2 of the biomarkers in TABLE 8. In another embodiment, biomarkers to improve identification or stratification of patients comprises at least 2 of the biomarkers in TABLE 9. In another embodiment, biomarkers to improve identification or stratification of patients comprises at least 2 of the biomarkers in TABLE 10. In another embodiment, biomarkers to improve identification or stratification of patients comprises at least 2 of the biomarkers in TABLE 11. In another embodiment, biomarkers to improve identification or stratification of patients comprises at least 2 of the biomarkers in TABLE 12. In another embodiment, biomarkers to improve identification or stratification of patients comprises at least 2 of the biomarkers in TABLE 13. In another embodiment, biomarkers to improve identification or stratification of patients comprises at least 2 of the biomarkers in TABLE 14. In another embodiment, biomarkers to improve identification or stratification of patients comprises at least 2 of the biomarkers in TABLE 15. In another embodiment, biomarkers to improve identification or stratification of patients comprises at least 2 of the biomarkers in TABLE 16. In another embodiment, biomarkers to improve identification or stratification of patients comprises at least 2 of the biomarkers in TABLE 17. In another embodiment, biomarkers to improve identification or stratification of patients comprises at least 2 of the biomarkers in TABLE 18. In another embodiment, biomarkers to improve identification or stratification of patients comprises at least 2 of the biomarkers in TABLE 19. In another embodiment, biomarkers to improve identification or stratification of patients comprises at least 2 of the biomarkers in TABLE 20. In another embodiment, biomarkers to improve identification or stratification of patients comprises at least 2 of the biomarkers in TABLE 21. In another embodiment, biomarkers to improve identification or stratification of patients comprises at least 2 of the biomarkers in TABLE 22. In another embodiment, biomarkers to improve identification or stratification of patients comprises at least 2 of the biomarkers in TABLE 23. In another embodiment, biomarkers to improve identification or stratification of patients comprises at least 2 of the biomarkers in TABLE 24. In another embodiment, biomarkers to improve identification or stratification of patients comprises at least 2 of the biomarkers in TABLE 25. In another embodiment, biomarkers to improve identification or stratification of patients comprises at least 2 of the biomarkers in TABLE 26. In another embodiment, biomarkers to improve identification or stratification of patients comprises at least 2 of the biomarkers in TABLE 27. In another embodiment, biomarkers to improve identification or stratification of patients comprises at least 2 of the biomarkers in TABLE 28. In another embodiment, biomarkers to improve identification or stratification of patients comprises at least 2 of the biomarkers in TABLE 29. In another embodiment, biomarkers to improve identification or stratification of patients comprises at least 2 of the biomarkers in TABLE 30. In another embodiment, biomarkers to improve identification or stratification of patients comprises at least 2 of the biomarkers in TABLE 31. In another embodiment, biomarkers to improve identification or stratification of patients comprises at least 2 of the biomarkers in TABLE 32.

In another embodiment, biomarkers to improve identification or stratification of patients comprises at least 2 of the biomarkers in TABLE 33.

In another embodiment, biomarkers to improve identification or stratification of patients comprises at least 10 of the biomarkers in TABLE 1. In another embodiment, biomarkers to improve identification or stratification of patients comprises at least 10 of the biomarkers in TABLE 2. In another embodiment, biomarkers to improve identification or stratification of patients comprises at least 10 of the biomarkers in in TABLE 3. In another embodiment, biomarkers to improve identification or stratification of patients comprises at least 10 of the biomarkers in TABLE 4. In another embodiment, biomarkers to improve identification or stratification of patients comprises at least 10 of the biomarkers in TABLE 5. In another embodiment, biomarkers to improve identification or stratification of patients comprises at least of the biomarkers in TABLE 6. In another embodiment, biomarkers to improve identification or stratification of patients comprises at least 10 of the biomarkers in TABLE 7. In another embodiment, biomarkers to improve identification or stratification of patients comprises at least of the biomarkers in TABLE 8. In another embodiment, biomarkers to improve identification or stratification of patients comprises at least 10 of the biomarkers in TABLE 9. In another embodiment, biomarkers to improve identification or stratification of patients comprises at least of the biomarkers in TABLE 10. In another embodiment, biomarkers to improve identification or stratification of patients comprises at least 10 of the biomarkers in TABLE 11. In another embodiment, biomarkers to improve identification or stratification of patients comprises at least 10 of the biomarkers in TABLE 12. In another embodiment, biomarkers to improve identification or stratification of patients comprises at least 10 of the biomarkers in TABLE 13. In another embodiment, biomarkers to improve identification or stratification of patients comprises at least 10 of the biomarkers in TABLE 14. In another embodiment, biomarkers to improve identification or stratification of patients comprises at least 10 of the biomarkers in TABLE 15. In another embodiment, biomarkers to improve identification or stratification of patients comprises at least 10 of the biomarkers in TABLE 16. In another embodiment, biomarkers to improve identification or stratification of patients comprises at least of the biomarkers in TABLE 17. In another embodiment, biomarkers to improve identification or stratification of patients comprises at least 10 of the biomarkers in TABLE 18. In another embodiment, biomarkers to improve identification or stratification of patients comprises at least 10 of the biomarkers in TABLE 19. In another embodiment, biomarkers to improve identification or stratification of patients comprises at least 10 of the biomarkers in TABLE 20. In another embodiment, biomarkers to improve identification or stratification of patients comprises at least 10 of the biomarkers in TABLE 21. In another embodiment, biomarkers to improve identification or stratification of patients comprises at least 10 of the biomarkers in TABLE 22. In another embodiment, biomarkers to improve identification or stratification of patients comprises at least 10 of the biomarkers in TABLE 23. In another embodiment, biomarkers to improve identification or stratification of patients comprises at least of the biomarkers in TABLE 24. In another embodiment, biomarkers to improve identification or stratification of patients comprises at least 10 of the biomarkers in TABLE 25. In another embodiment, biomarkers to improve identification or stratification of patients comprises at least 10 of the biomarkers in TABLE 26. In another embodiment, biomarkers to improve identification or stratification of patients comprises at least 10 of the biomarkers in TABLE 27. In another embodiment, biomarkers to improve identification or stratification of patients comprises at least 10 of the biomarkers in TABLE 28. In another embodiment, biomarkers to improve identification or stratification of patients comprises at least 10 of the biomarkers in TABLE 29. In another embodiment, biomarkers to improve identification or stratification of patients comprises at least 10 of the biomarkers in TABLE 30. In another embodiment, biomarkers to improve identification or stratification of patients comprises at least of the biomarkers in TABLE 31. In another embodiment, biomarkers to improve identification or stratification of patients comprises at least 10 of the biomarkers in TABLE 32.

In another embodiment, biomarkers to improve identification or stratification of patients comprises at least 10 of the biomarkers in TABLE 33.

A gene signature can result from the measurement of expression of the RNA and/or the protein expressed by the gene corresponding to the biomarkers of Table 1 and/or Tables 2-32 of the invention. In the case of RNA it refers to the RNA transcripts transcribed from genes corresponding to the biomarkers of the invention. In the case of protein, it refers to proteins translated from the genes corresponding to the biomarkers of the invention. For example, techniques to measure expression of the RNA products of the biomarkers of the invention include PCR based methods (including RT-PCR) and non-PCR based methods as well as microarray analysis. To measure protein products of the biomarkers of the invention, techniques include western blotting and ELISA analysis, and proteomic profiling (e.g., Mass Spectrometry, Imaging Mass Cytometry (histo-CyTOF, etc.).

The inventive assays include assays both to select patients eligible to receive cancer therapy and assays to monitor patient response. These assays can be performed by protein assay methods and by nucleic acid assay methods. Any type of either protein or nucleic acid assays can be used. Protein assay methods useful in the invention are well known in the art and comprise (i) immunoassay methods involving binding of a labeled antibody or protein to the expressed protein or fragment thereof, (ii) mass spectrometry methods to determine expressed protein or fragments of these biomarkers, and (iii) proteomic based or “protein chip” assays. Useful immunoassay methods include both solution phase assays conducted using any format known in the art, such as, but not limited to, an ELISA format, a sandwich format, a competitive inhibition format (including both forward or reverse competitive inhibition assays) or a fluorescence polarization format, and solid phase assays such as immunohistochemistry (referred to as “IHC”).

IHC is a method of detecting the presence of specific proteins in cells or tissues and consists of the following steps: 1) a slide is prepared with the tissue to be interrogated; 2) a primary antibody is applied to the slide and binds to specific antigen; 3) the resulting antibody-antigen complex is bound by a secondary, enzyme-conjugated, antibody; 4) in the presence of substrate and chromogen, the enzyme forms a colored deposit (a “stain”) at the sites of antibody-antigen binding; and 5) the slide is examined under a microscope to identify the presence of and extent of the stain.

Nucleic acid assay methods useful in the invention are also well known in the art and comprise (i) in situ hybridization assays to intact tissue or cellular samples to detect mRNA levels or chromosomal DNA changes, (ii) microarray hybridization assays to detect mRNA levels or chromosomal DNA changes, (iii) RT-PCR assays or other amplification assays to detect mRNA levels or (iv) PCR or other amplification assays to detect chromosomal DNA changes. Assays using synthetic analogs of nucleic acids, such as peptide nucleic acids, in any of these formats can also be used.

The invention provides a method to identify altered expression levels of the genes in Pan-Signature (Table 1), or a subset thereof, for both response prediction and for monitoring patient response to cancer therapy. Assays for response prediction are run before therapy selection and a sample determined as having at least one (1), or at least two (2), or at least ten (10) differentially expressed genes from the Pan-Signature and/or a sub-signature and/or a cancer specific signature list compared to controls as defined herein, and classified as having a high or low FMRP activity score as the case may be, would be eligible to receive a particular cancer therapy judged to be differentially responsive as a function of FMRP activity.

For monitoring patient response to FMRP inhibitors, the assay could be run at the initiation of therapy to establish the FMRP activity score and the baseline levels of the genes in the tissue sample. The same tissue is then sampled and assayed and the levels of the genes are compared to the baseline. Where the levels remain the same or decrease, the therapy is likely being effective and can be continued. Where significant increase over baseline level occurs, the patient may not be responding.

As used herein, cancer therapy includes, but is not limited to, treatment with one or more inhibitors of FMRP protein expression or activity, treatment with one or more immune checkpoint inhibitors, chemotherapy treatment, radiation, targeted cancer therapy, or combinations thereof. In embodiments, cancer therapy includes, but is not limited to, treatment with an inhibitor of FMRP protein expression or activity, treatment with an immune checkpoint inhibitor, chemotherapy treatment or combinations thereof. In embodiments, the cancer therapy is treatment with inhibitors of FMRP protein expression or activity. In embodiments, cancer therapy is treatment with an immune checkpoint inhibitor. In embodiments, cancer therapy is chemotherapy treatment.

As used herein, the term “in combination” when referring to therapeutic treatments refers to the use of more than one type of therapy. The use of the term “in combination” does not restrict the order in which therapies are administered to a subject. Such combination may also include more than a single administration of a therapy. The administration of the therapies may be by the same or different routes. The one or more therapies can be co-administered. The terms “co-administered” or “co-administration” generally refers to the administration of at least two different substances sufficiently close in time. Co-administration refers to simultaneous administration, as well as temporally spaced order of up to several days apart, of at least two different substances in any order, either in a single dose or separate doses.

Checkpoint inhibitors include, but are not limited to, anti-PD1, anti-PDL1 and anti-CTLA inhibitors (antibodies). In embodiments, the checkpoint inhibitor is an anti-CTLA-4 antagonist antibody such as ipilimumab, tremelimumab, and BMS-986249. In embodiments, the checkpoint inhibitor is an anti-PD-1 or anti-PD-L1 antagonist antibody such as avelumab, atezolizumab, CX-072, pembrolizumab, nivolumab, cemiplimab, spartalizumab, tislelizumab, JNJ-63723283, genolimzumab, AMP-514, AGEN2034, durvalumab, and JNC-1.

Chemotherapeutic agents include, but are not limited to, afatinib, capecitabine, carboplatin, cisplatin, cobimetanib, crizotinib, cyclophosphamide, dabrafenib, dacarbazine, dexamethasone, docetaxel, doxorubicin, daunorubicin, epirubicin, eribulin, erlotinib, etoposide, fludarabine, 5-FU, gemcitabine, gefitinib, irinotecan, ixabepilone, CHOP (C: CYTOXAN® (cyclophosphamide); H: ADIAMYCIN® (hydroxydoxorubicin); O: Vincristine (ONCOVIN®); P: prednisone), methotrexate, mitoxantrone, oxaliplatin, paclitaxel, nab-paclitaxel, pemetrexed, rapamycin, RITUXIN® (rituximab), temozolomide, trametinib, vemurafenib, vinorelbine, and vincristine.

Targeted therapies include, but are not limited to, EGFR, ALK, ROS, RAS, BRAF, or BCL2.

In any of the embodiments herein, if the cancer therapy is an FMRP inhibitor, one might choose tumors with a high FMRP activity score. In any of the embodiments herein, if the cancer therapy is an immune checkpoint inhibitor and/or a chemotherapy, one might select patients with a low FMRP activity score, unless the therapy was combined with an FMRP inhibitor.

In embodiments, cancer includes, but is not limited to, AML (acute myeloid leukemia), BRCA (breast cancer), CCC (cholangiocellular carcinoma), CLL (chronic lymphocytic leukemia), CRC (colorectal cancer), GBC (gallbladder cancer), GBM (glioblastoma), GC (gastric cancer), GEJC (gastro-esophageal junction cancer), HCC (hepatocellular carcinoma), HNSCC (head and neck squamous cell carcinoma), MEL (melanoma), NHL (non-Hodgkin lymphoma), NSCLC (non-small cell lung cancer), OC (ovarian cancer), OSCAR (esophageal cancer), PACA (pancreatic cancer), PRCA (prostate cancer), RCC (renal cell carcinoma), SCLC (small cell lung cancer), UBC (urinary bladder carcinoma), and UEC (uterine endometrial cancer). In embodiments, cancer includes, but is not limited to, gastric cancer, breast cancer, which optionally is triple negative breast cancer (TNBC), non-small cell lung cancer (NSCLC), melanoma, renal cell carcinoma (RCC), bladder cancer, endometrial cancer, diffuse large B-cell lymphoma (DLBCL), Hodgkin's lymphoma, ovarian cancer, and head and neck squamous cell cancer (HNSCC).

In any of the embodiments herein, the biomarkers and signature lists of the invention are useful for cancer in general and Adrenocortical carcinoma, Bladder Carcinoma, Breast Carcinoma, Cervical Carcinoma, Colon adenocarcinoma, Esophageal carcinoma, Glioblastoma, Head and Neck carcinoma, Kidney Chromophobe, Kidney renal clear cell carcinoma, Kidney renal papillary cell carcinoma, Acute Myeloid Leukemia, Glioma, Hepatocellular carcinoma, Lung Adenocarcinoma, Lung squamous cell carcinoma, Ovarian Carcinoma, Pancreatic adenocarcinoma, Pheochromocytoma and Paraganglioma, Prostate adenocarcinoma, Rectum adenocarcinoma, Sarcoma, Melanoma, Stomach adenocarcinoma, Testicular Tumors, Thyroid carcinoma, Thymoma, or Endometrial Carcinoma in particular.

The invention comprises diagnostic assays performed on a patient sample (also referred to as the “sample”, “tissue sample”, or “query sample”) of any type or on a derivative thereof, including peripheral blood, tumor or suspected tumor tissues (including fresh frozen and fixed or paraffin embedded tissue), cell isolates such as circulating epithelial cells separated or identified in a blood sample, lymph node tissue, bone marrow and fine needle aspirates. Preferred samples for use herein are peripheral blood, tumor or suspected tumor tissue and bone marrow.

Furthermore, this invention provides for cell-based assays involving cancer cells expressing high levels of FMRP protein and its gene signature of pathway activity, to be used in identifying and/or validating inhibitors of said FMRP activity. Such activity-inhibition assays can be powerful tools when applied to screening efforts aimed at discovering and developing pharmaceuticals targeting FMRP and/or FMRP's immunosuppressive and pro-invasive/pro-metastatic pathways. As for diagnostic applications, such cell-based assays could use mRNA or protein representing the signature genes.

### EXAMPLES

The present invention was developed using mouse cancer cell lines and tumors alternatively expressing or lacking expression of FMRP due to genetic ablation of the FMR1 gene. Importantly, the identified biomarkers and the method to develop a signature score reporting on FMRP pathway activity is demonstrably applicable across multiple human cancer types and can be used to predict prognosis of cancer patients in various tumor types.

The invention demonstrates that the FMRP-activity signature score is capable of predicting which patients will benefit from FMRP inhibitor therapies. The present invention would represent a companion diagnostic for ‘precision medicine’ strategies that reveal the degree of FMRP's pathway activity and inferred immunosuppressive capability so as to more accurately select patients who would most likely respond to potential inhibitors of FMRP.

In addition, the invention demonstrates that the FMRP-activity signature score is capable of predicting which patients will benefit from immune checkpoint inhibitor therapies. Therefore, the identified biomarkers and the corresponding method can be used alongside and/or in addition to the current biomarkers for classifying patients for treatment or not with immunotherapies. The FMRP activity score may also be applicable to clinical decisions to treat cancer patients with other therapeutic modalities involving an adaptive immune response, as illustrated for chemotherapies.

### Example 1—Developing FMRP-Activity Signatures

The current invention is based on two separate experiments applying state-of-art gene knock-out systems that have been implemented both in-vitro (cell culture) and in-vivo (tumor-bearing mice). Bulk and single-cell RNA-sequencing techniques were used to measure gene expression levels, as well as sophisticated bioinformatic analyses to establish gene-list and corresponding methods to develop signature scores representing FMRP pathway-activity in cancer cells.

FMR1 (the gene encoding for FMRP protein) was genetically deleted in a mouse pancreatic cancer cell line by employing the CRISPR-Cas9 system to target the deletion of the essential first exon in the FMR1 gene. In the first model, cancer cells in culture were subject to RNA-sequencing analysis, and differentially expressed genes (fold change >1.5) were identified, comparing isogenic cell lines in which the FMR1 gene was intact and its gene product FMRP was expressed (FMRP-WT) and a derivative in which the FMR1 was deleted and FMRP was not expressed (FMRP-KO). This list of significantly differentially expressed genes defines a “signature” consisting of the genes that FMRP regulates, directly or indirectly, in cancer cells that express it; this gene set is dubbed the FMRP-Activity “Sub-Signature 1”. In the second model, FMRP-WT and FMRP-KO cancer cells were inoculated (subcutaneous) into immunocompetent mice, and solid tumors allowed to form. Tumors were excised and subjected to single-cell RNA-sequencing analysis, and subsequently, differentially expressed genes (fold change >1.5) between FMRP-WT and FMRP-KO tumors were identified, defining a second gene set, dubbed FMRP-Activity “Sub-Signature 2”. The union of these two differentially-expressed gene lists constitute and define FMRP-Activity “Pan-Signature”. Additionally, genes reflecting an indirect innate-immune response in the tumors, annotated from the Gene-ontology signature list, were excluded from Pan-Signature, and the remaining genes define FMRP-Activity “Sub-Signature 3”. Additionally, derivative cancer-type specific signatures were developed by using the COX model and sub-selecting the genes from Pan-Signature, including only those genes that collectively show a significant correlation with overall and progression-free survival in the TCGA cohort of a particular cancer type (Hazard ratio >1.2).

### Example 2

Tumors samples from TCGA, after inferring the signature scores, were classified based on signature score quantiles: FMRP-low (samples with score <Q1), FMRP-median (samples with scores between Q1 and Q3), FMRP-high (samples with scores larger than Q3). Kaplan-Meier survival analysis was used to assess the relationship of the signature scores with survival. COX model was used to determine the associations between predictor variables and to obtain adjusted hazard-ratios. The tumor types were included as co-variates in the COX model.

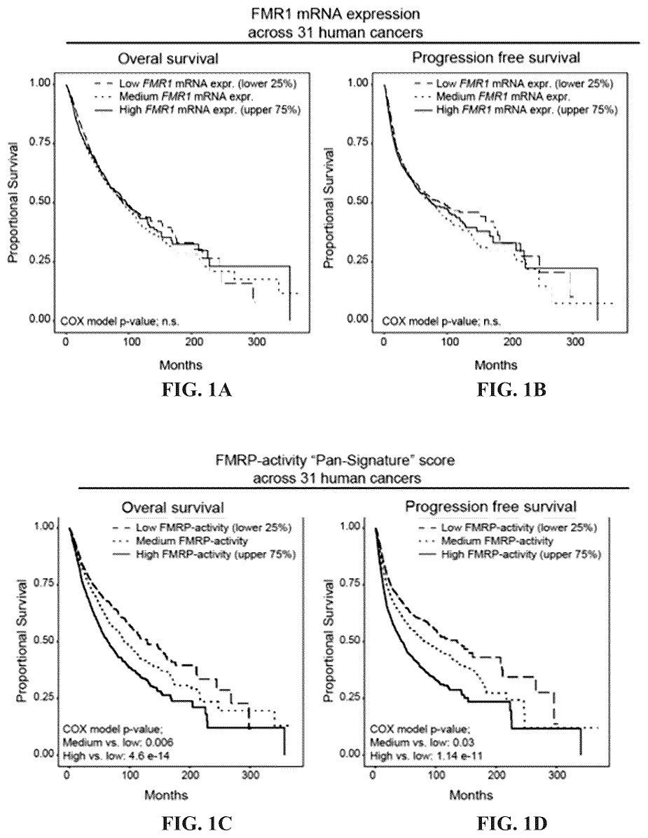

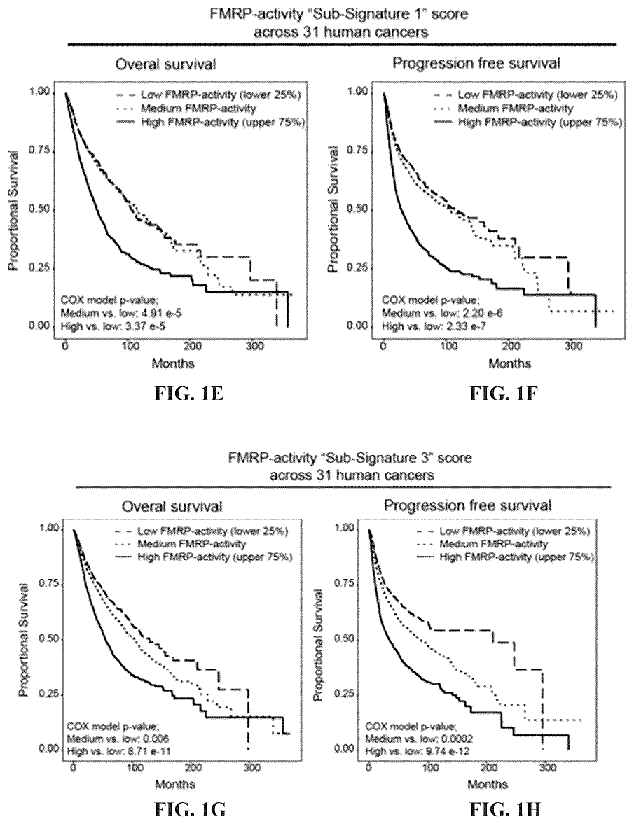

FIG. 1 shows patient classification across 31 different cancer types, based on FMR1 mRNA expression (panels A and B), which is not informative, in contrast to the newly invented FMRP pathway-activity signature score (FMRP-activity Pan-Signature: panels C and D; Sub-Signature 1: Panels E and F; Sub-Signature 3: Panels G and H), which it is informative and statistically significant for all. Each panel shows the association (or not) with patient prognosis (A, C, E, and G: overall survival; B, D, F, and H: progression-free survival). The COX-model was used, considering the tumor type as covariate, to estimate the significance of correlation. The data used in this figure were downloaded from the latest TCGA PanCan Atlas.

### Example 3

The application of the classification method according to the invention is applied for two different cancer types,—breast cancer and colorectal cancer—in which FMRP has been implicated. The use of the current invention to predict patients' response to immune checkpoint inhibitors as well as to chemotherapy in several cancer types is demonstrated. For these analyses Pan-Signature was used unless otherwise mentioned in the legend.

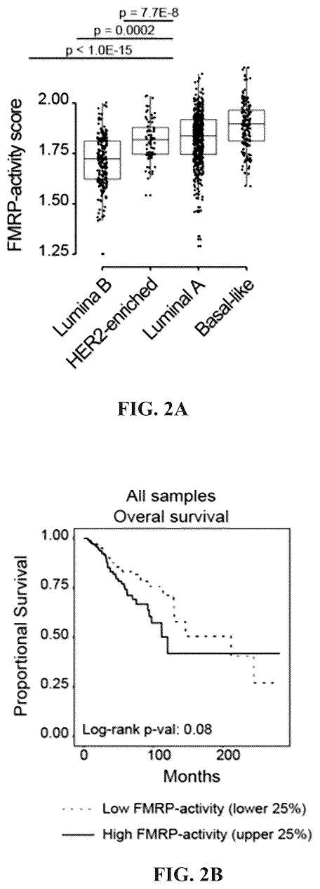

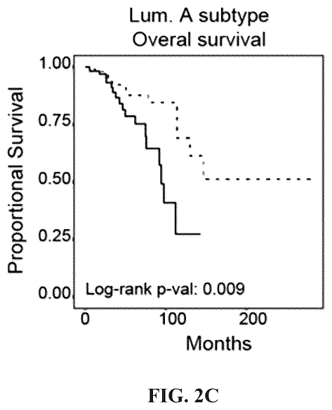

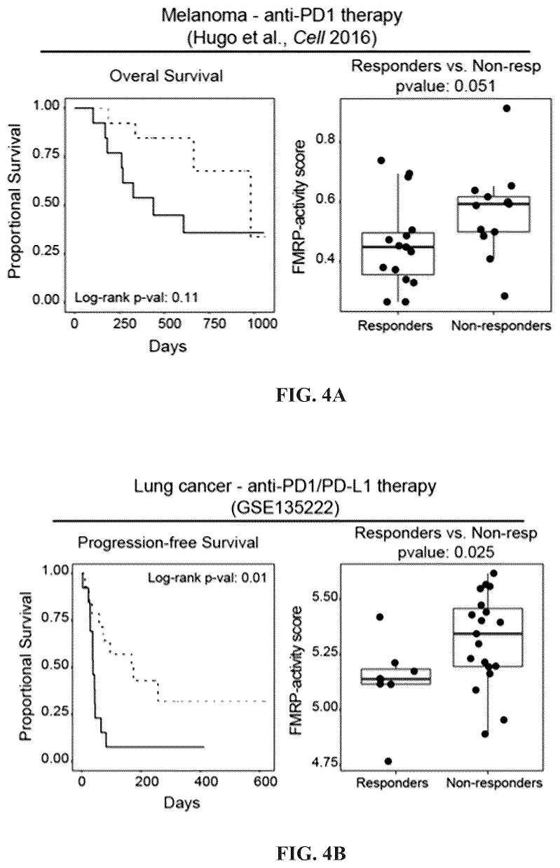

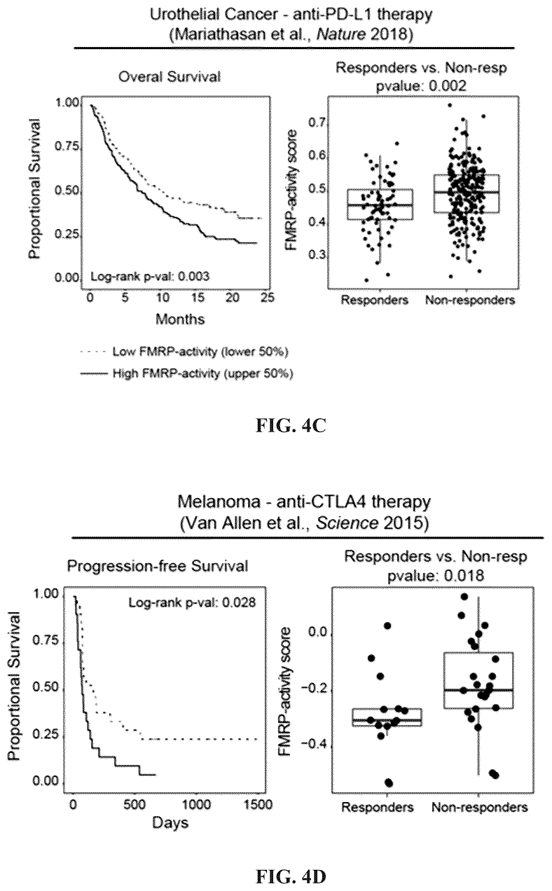

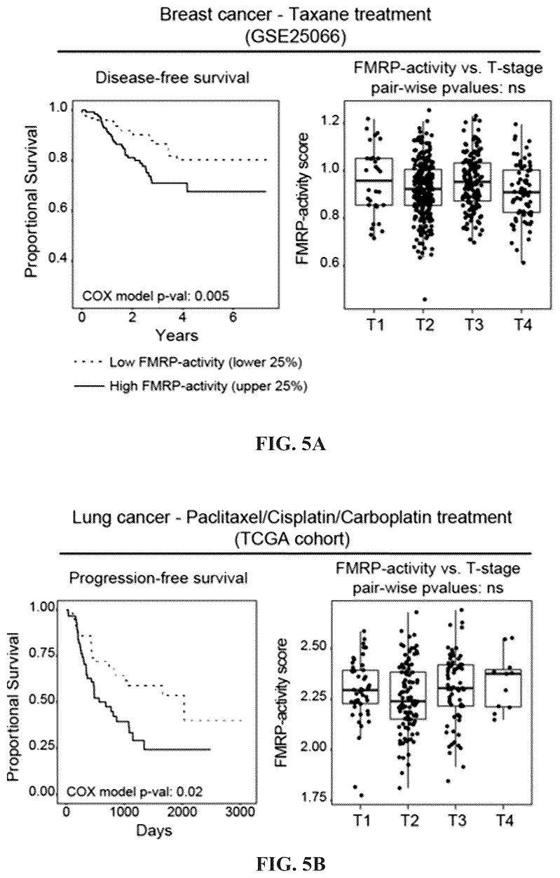

FMRP signature scores for each tumor sample were developed as described above. For survival analysis, similar to FIG. 1 discussed above, samples were classified based on signature scores (for FIG. 2/3/5: low score <Q1 and high score >Q3; for FIG. 4: low score <Q2 and high score >Q2, as shown within the figures). For the Boxplots (correlation analysis) the signature scores in each subtype were compared and tested for significant difference using Wilcoxon test. Subtypes used for each figure is as follows; subtypes for FIG. 2: Breast cancer PAM50 subtypes; subtypes for FIG. 4: responders and non-responders; subtypes for FIG. 5: tumor T-stages.

FIG. 2: FMRP-activity score in breast cancer. FIG. 2A. The FMRP-activity score shows the highest level in the basal-like subtype, which is the most aggressive subtype of breast cancer. Only up-regulated genes in Pan-Signature were used to derive the signature scores for this panel. FIG. 2B. The FMRP-activity score correlates with overall survival for all breast cancer patients. FIG. 2C. The FMRP-activity score specifically correlates with overall survival for the Luminal A subtype of breast cancer patients. The data used in this figure were downloaded from the latest breast cancer cohort of TCGA PanCan Atlas.

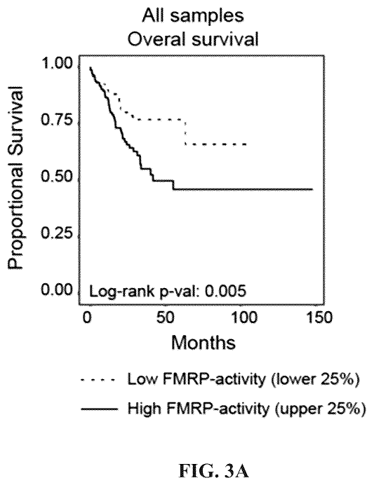

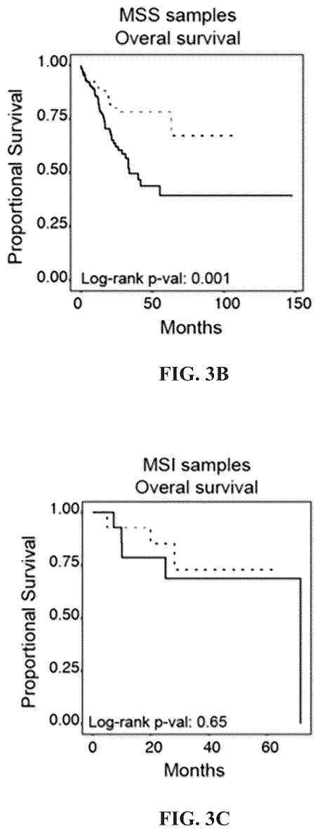

FIG. 3 depicts FMRP-activity score in colorectal carcinoma. FIG. 3A. FMRP-activity score correlation with overall survival for all colorectal cancer patients. FIG. 3B. FMRP-activity score correlation with overall survival for microsatellite stable (MSS) colorectal cancer patients. FIG. 3C. shows a lack of correlation of the FMRP-activity score with overall survival for microsatellite instable (MSI) colorectal cancer patients. The data used in this figure were downloaded from the latest colorectal cancer cohort of TCGA PanCan Atlas.

FIG. 4 depicts FMRP-activity score correlation with immune-checkpoint inhibitor therapy response in cancer patients. FIG. 4A. FMRP-activity score correlation with overall survival for melanoma patients receiving anti-PD1 therapy (left panel); non-responders to anti-PD1 therapy show a higher level of the FMRP-activity score (right panel). FIG. 4B. FMRP-activity score correlation with overall survival for lung cancer patients receiving anti-PD1 or anti-PD-L1 therapy (left panel); non-responders to anti-PD1 or anti-PD-therapy show a higher level of the FMRP-activity score (right panel). FIG. 4C. FMRP-activity score correlation with overall survival for urothelial cancer patients receiving anti-PD-L1 therapy (left panel); non-responders to anti-PD-L1 therapy show a higher level of the FMRP-activity score (right panel). Only up-regulated genes in Sub-Signature 1 were used to derive the signature scores for panel A-C. FIG. 4D. FMRP-activity score (Pan-Signature) correlation with overall survival for melanoma patients receiving anti-CTLA4 therapy (left panel); non-responders to anti-CTLA4 therapy show a higher level of the FMRP-activity score (right panel).

FIG. 5 depicts FMRP-activity score correlation with chemotherapy response in cancer patients. FIG. 5A. FMRP-activity score correlation with disease-free survival for breast cancer patients receiving Taxanes (left panel); notably, the signature scores are independent of tumor aggressiveness (T-stage, right panel), also shown using COX model in survival analysis considering the T-stage as covariate, which therefore reveals that FMRP-activity signature constitutes an independent prognostic marker. FIG. 5B. shows FMRP-activity score correlation with progression-free survival for lung cancer patients receiving Paclitaxel, Cisplatin, or Carboplatin (left panel); the signature scores are again independent of tumor aggressiveness (T-stage, right panel), constituting an independent prognostic factor. The COX-model was used, considering the T-stage as covariate, to estimate the significance of correlation for survival analysis. Only up-regulated genes from Sub-Signature 1 were used to derive the signature scores for all the panels.

### Example 4

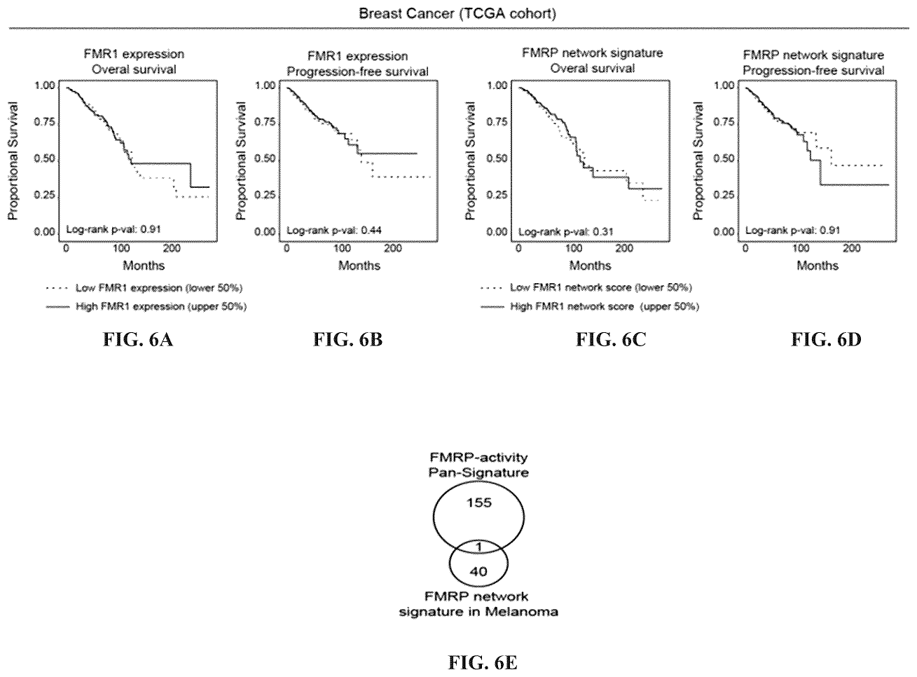

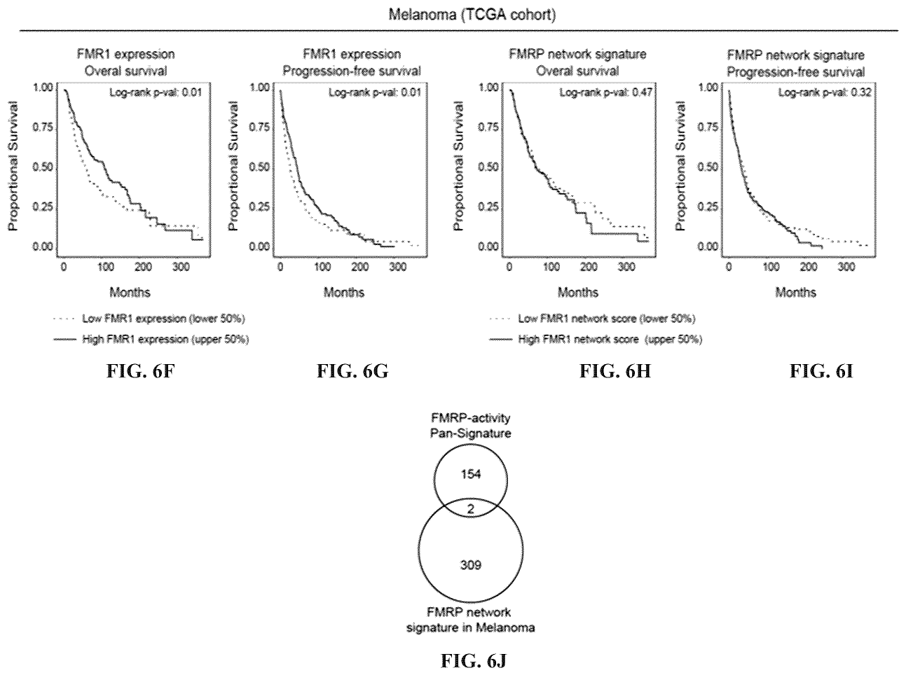

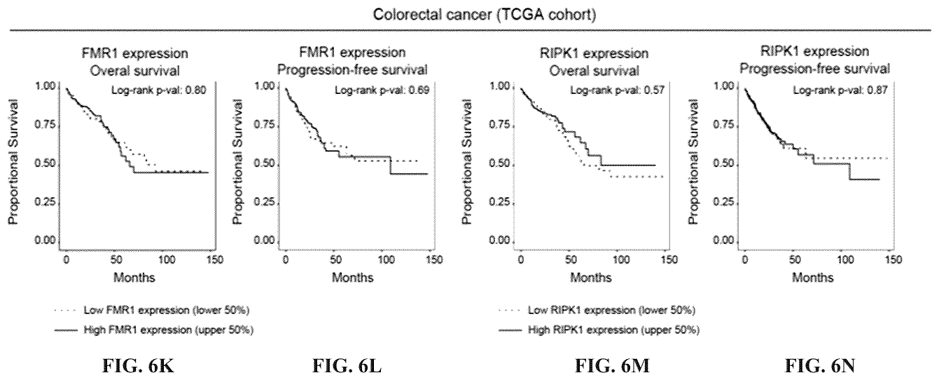

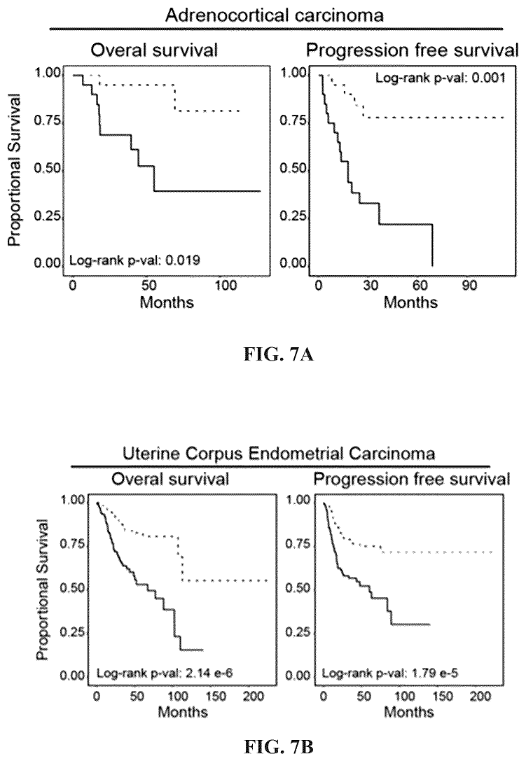

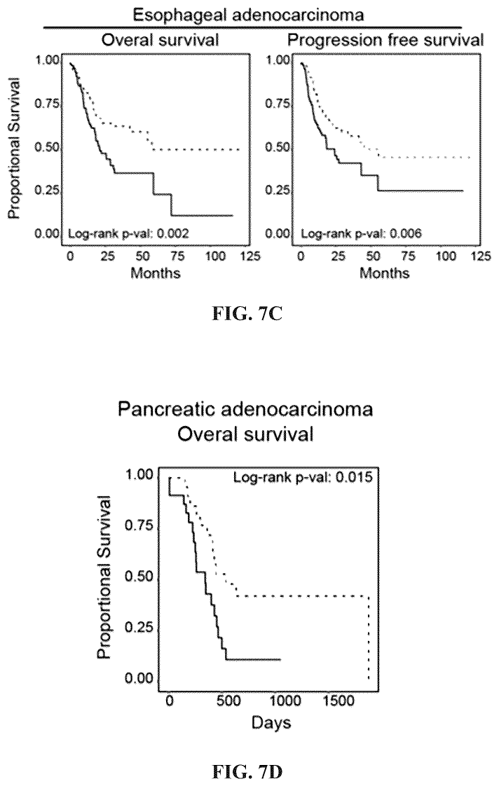

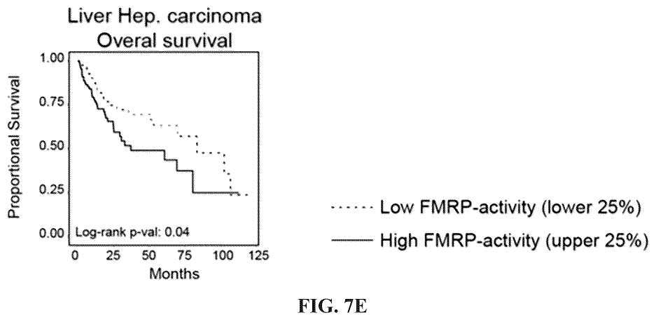

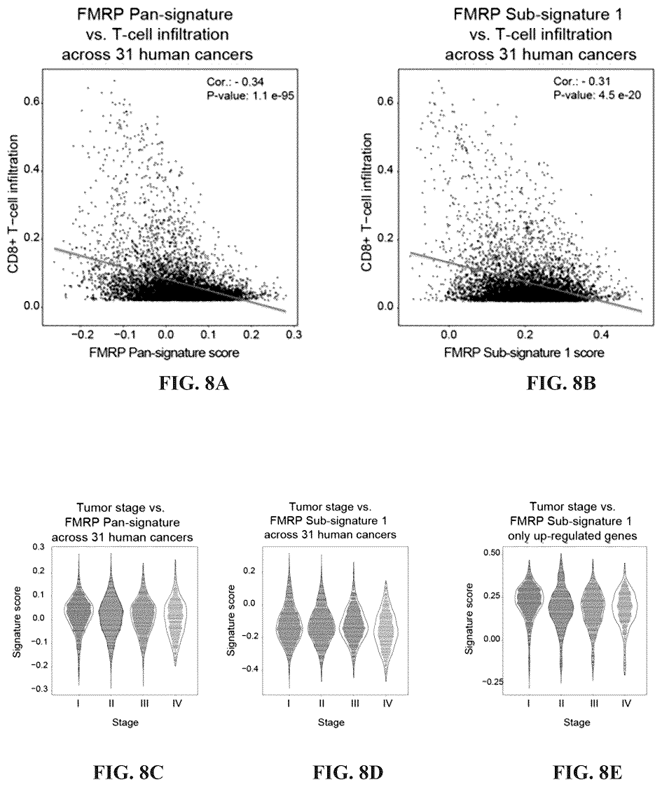

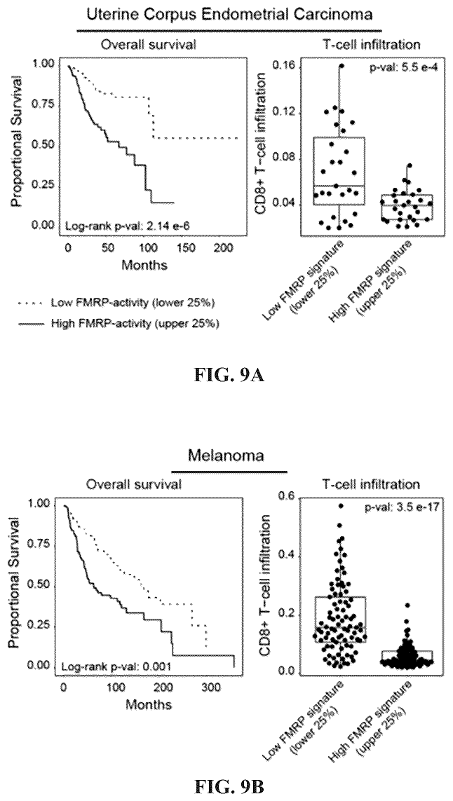

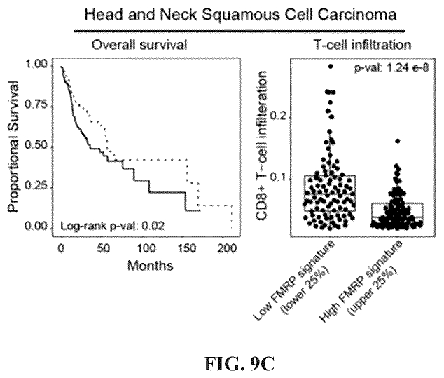

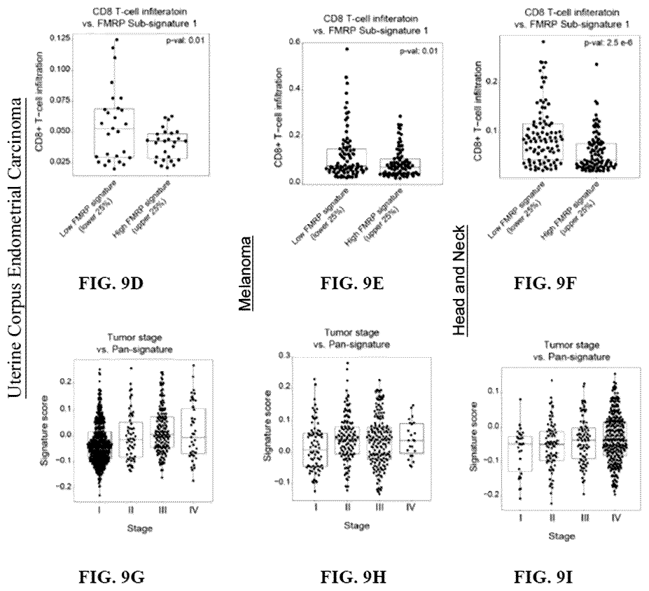

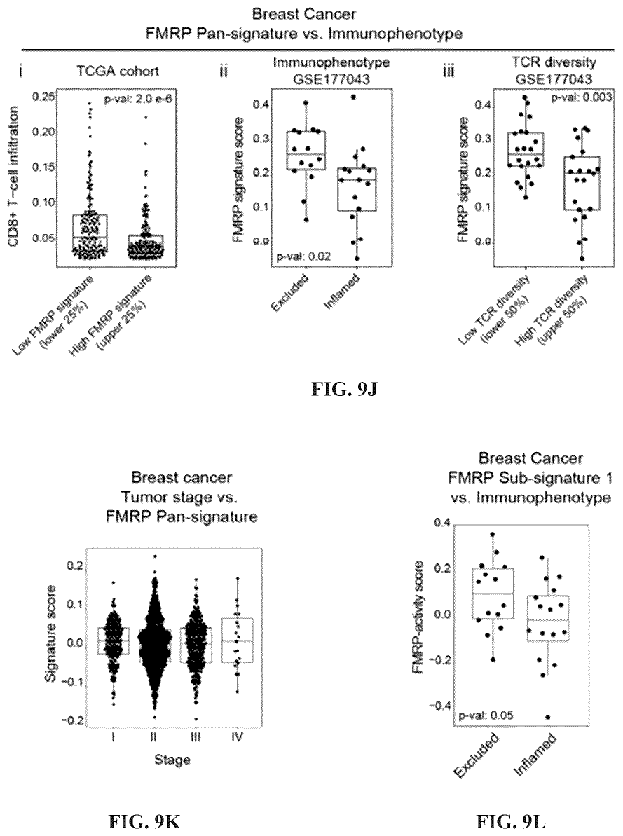

FIG. 6 shows the non-reproducibility and lack of Correlation between previously published FMRP signatures and those described in this invention. FMR1 mRNA expression (FIG. 6A and FIG. 6B), and FMRP network signature (Luca et al., (2013), FIG. 6C and FIG. 6D) correlations with Breast cancer patients' survival are not informative or statistically significant. Each panel shows the association (or not) with patient prognosis (FIG. 6A, FIG. 6C: overall survival; FIG. 6B, FIG. 6D: progression-free survival). FIG. 6E. Genes constituting the FMRP network signature proposed by Rossella Luca et al., 2013 show no significant overlap with Pan-Signature 1 described in this invention. FMR1 mRNA expression (FIG. 6F and FIG. 6G), and FMRP network signature (Zalfa et al., (2017), FIG. 6H and FIG. 6I) correlations with melanoma patients' survival again are not informative or statistically significant. Each panel shows patient prognosis (FIG. 6F, FIG. 6H: overall survival; FIG. 6G, FIG. 6L progression-free survival). FIG. 6J. The genes comprising the FMRP network signature proposed by F. Zalfa et al., 2017 show no significant overlap with Pan-Signature provided in this invention. FMR1 mRNA expression (FIG. 6K and FIG. 6L), and RIPK1 mRNA expression (FIG. 6M and FIG. 6N) correlations with colorectal cancer patients' survival again are not informative or statistically significant. Each panel shows patient prognosis (FIG. 6K, FIG. 6M: overall survival; FIG. 6L, FIG. 6N: progression-free survival).

### Example 5

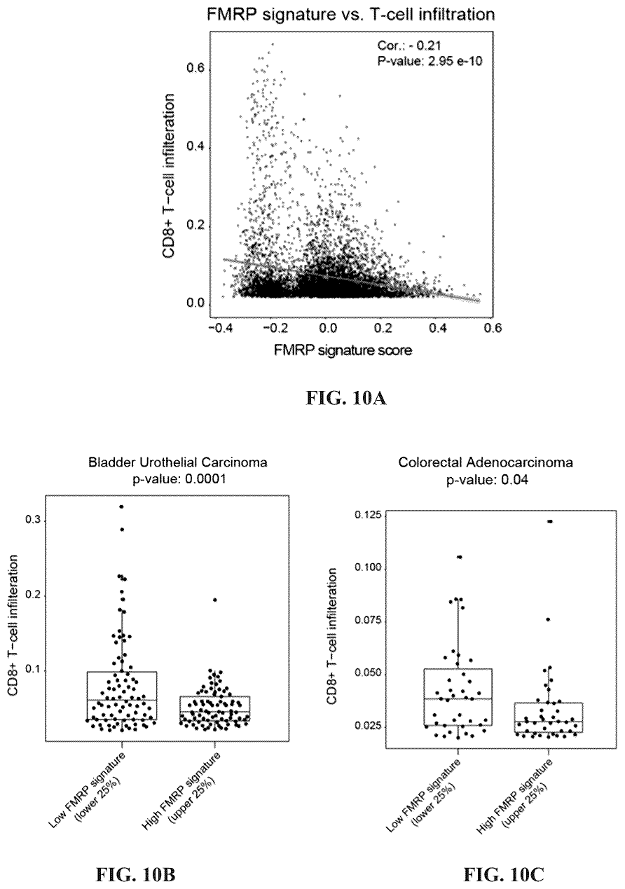

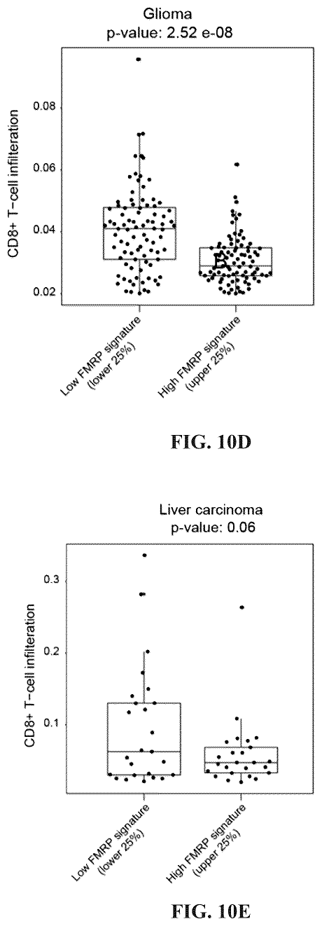

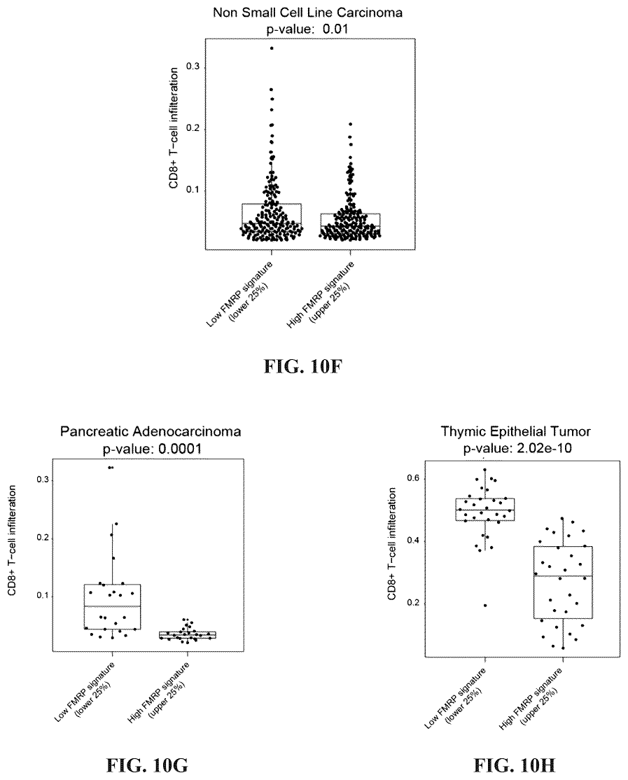

Murrin PDAC cancer cell line was transfected with siRNA targeting FMR1 mRNA, which results in significant knock-down of the FMRP expression. After 24 hours of transfection with siFMRP and siControl (which does not target any mRNA), the cells were subjected to RNA-seq analysis, and subsequently, the signature were developed based on up-regulated genes in siCTRL vs. siFMRP cancer cells. FIG. 10 shows the inverse correlation in the level of tumor inflammation with CD8 T-cell for this Pan-Immunosupressive signature, reflecting its capability to suppress T cell inflammation.

While this invention has been particularly shown and described with references to preferred embodiments thereof, it will be understood by those skilled in the art that various changes in form and details may be made therein without departing from the scope of the invention encompassed by the appended claims.

1. A method for identifying a patient with cancer as (i) being high or low for FMRP activity, (ii) having a high or low risk prognosis and/or (iii) being a responder or non-responder to cancer therapy comprising:
(a) obtaining a sample from the patient;
(b) determining expression level for the genes in one or more of the signatures set forth in Tables 1 through 32 in the sample;
(c) comparing the expression levels in step (b) relative to the level of said genes expressed in a control;
(d) identifying the differentially expressed gene(s) between the sample and control; and
(e) classifying the patient as (i) being high or low for FMRP activity, (ii) having a high or low risk prognosis and/or (iii) being a responder or non-responder to cancer therapy based on the concordance of the differential expression with the one or more signatures.

2. The method according to claim 1, wherein the gene expression level is determined in one signature set forth in Tables 1 through 32.

3. The method according to claim 1, wherein the gene expression level is determined in two or more signatures set forth in Tables 1 through 32.

4. The method according to claim 1, wherein at least one gene in the one or more signatures is differentially expressed relative to the control.

5. The method according to claim 1, wherein at least ten (10) genes in the one or more signatures are differentially expressed relative to the control.

6. The method according to claim 1, wherein the method identifies the patient as being high or low for FMRP activity.

7. The method according to claim 1, wherein the method identifies the patient as having a high or low risk prognosis.

8. The method according to claim 1, wherein the method identifies the patient as being a responder or non-responder to cancer therapy.

9. The method according to claim 1, wherein the patient sample is a blood or other bodily fluid.

10. The method according to claim 1, wherein the patient sample is a tissue sample.

11. The method according to claim 1, further comprising administering a cancer therapy to the patient of step (e).

12. The method according to claim 11, wherein the cancer therapy is an immune-checkpoint inhibitor; an anti-FMRP therapy, chemotherapy, radiotherapy, targeted therapy, or combinations thereof.

13. The method according to claim 11, wherein the cancer therapy is anti-FMRP therapy.

14. The method according to claim 13, further comprising administering an immune-checkpoint inhibitor and/or chemotherapy in combination with the FMRP inhibitor.

15. A method for stratifying a group of patients with cancer as (i) being high or low for FMRP activity, (ii) having a high or low risk prognosis and/or (iii) being a responder or non-responder to cancer therapy comprising:
(a) obtaining a sample from each patient of the group;
(b) determining expression level of the genes in one or more of the signatures set forth in Tables 1 through 32 for each sample;
(c) establishing an FMRP activity score for each sample; and
(d) classifying each patient as (i) being high or low for FMRP activity, (ii) having a high or low risk prognosis and/or (iii) being a responder or non-responder to cancer therapy based on the FMRP activity score.

16-28. (canceled)

29. A method for treating a patient with cancer comprising:
(a) obtaining a sample from the patient;
(b) determining expression level for the genes in one or more of the signatures set forth in Tables 1 through 32 in the sample;
(c) comparing the expression levels in step (b) relative to the level of said genes expressed in a control;
(d) identifying the differentially expressed gene(s) between the sample and control;
(e) classifying the patient as (i) being high or low for FMRP activity, (ii) having a high or low risk prognosis and/or (iii) being a responder or non-responder to cancer therapy based on the concordance of the differential expression with the signatures; and
(f) administering a cancer therapy to the patient.

30-34. (canceled)

35. A method for treating a group of patients with cancer comprising:
(a) obtaining a sample from each patient of the group;
(b) determining expression level of the genes in one or more of the signatures set forth in Tables 1 through 32 for each sample;
(c) establishing an FMRP activity score for each sample;
(d) classifying each patient as (i) being high or low for FMRP activity, (ii) having a high or low risk prognosis and/or (iii) being a responder or non-responder to cancer therapy based on the FMRP activity score; and
(e) administering a cancer therapy to each patient.

36-40. (canceled)

41. A method for predicting T-cell infiltration, the method comprising:
(a) obtaining a tumor sample (biopsy, resection) from the patient;
(b) determining expression level for the genes set forth in Table 33 in the sample;
(c) comparing the expression levels in step (b) relative to the level of said genes expressed in a control;
(d) identifying the differentially expressed gene(s) between the tumor sample and control; and
(e) classifying the patient as (i) being high or low for FMRP immunosuppressive activity, and (ii) having a high or low immune cell infiltration based on the concordance of the differential expression with the signature.

42-45. (canceled)
